(19) United States
(12) Patent Application Publication
(10) Pub. No.: US 2023/0002806 A1
(43) Pub. Date: Jan. 5, 2023

(54) Detecting Congenital Heart Defect

(71) Applicant: Bioscreening & Diagnostics LLC, Detroit, MI (US)

(72) Inventor: Ray Bahado-Singh, Grosse Pointe Shores, MI (US)

(73) Assignee: Bioscreening & Diagnostics LLC, Detroit, MI (US)

(21) Appl. No.: 17/756,610

(22) PCT Filed: Nov. 25, 2020
(86) PCT No.: PCT/US2020/062194

§ 371 Date: May 27, 2022

Related U.S. Application Data
(60) Provisional application No. 62/941,357, filed on Nov. 27, 2019.

(51) Int. Cl. C12Q 1/686; C12Q 1/6883

(52) U.S. Cl. CPC C12Q 1/686; C12Q 1/6883; C12Q 2600/154

(57) ABSTRACT

The present disclosure describes a method of detecting, diagnosing, or predicting congenital heart defect (CHD). The method is a primarily minimally invasive method, as it uses a biological sample from a subject for detecting methylation changes in the nucleic acids of the subject. The method also involves the use of artificial intelligence (AI).

### CROSS-REFERENCE TO RELATED APPLICATIONS

This application claims the benefit of U.S. Provisional Patent Application 62/941,357, filed on Nov. 27, 2019, which is hereby incorporated by reference in its entirety.

### TECHNICAL FIELD

The present disclosure relates to methods and systems for predicting and detecting congenital heart defects in patients using molecular markers.

### BACKGROUND

Epigenetic changes including DNA methylation are known to be involved in cardiac embryogenesis (O'Meara & Lee 2015) and in the development of congenital heart defects (Bahado-Singh et al. 2005; Bahado-Singh et al. 2016; Radhakrishna et al. 2016; Radhakrishna et al. 2019). With very few exceptions, cardiac tissue is not available for research analysis in living subjects. This is particularly the case for the developing fetus and this reality has retarded progress on the evaluation of epigenetic changes that are causal or linked to the development of congenital heart defect (CHD). As a consequence, there is significant interest in developing molecular markers in tissues such as blood leukocytes that also reflect epigenetic changes and are biologically linked or correlated with those in sequestered organs such as the developing heart. Many studies (Bahado-Singh et al. 2005; Bahado-Singh et al. 2016; Radhakrishna et al. 2016; Radhakrishna et al. 2019) have shown that the above objective might be achievable. Methylation of newborn leukocyte DNA obtained by heel stick has been used in these studies to elucidate the mechanisms of multiple different types of the common non-syndromic CHD. Also, using conventional statistical analysis and also Artificial Intelligence (AI) approaches, leukocyte DNA methylation accurately predicted different types of non-syndromic congenital heart defects (CHDs) (Bahado-Singh et al. 2019b).

Extensive publications, both clinical and laboratory (Burton & Jauniaux 2018; Maslen 2018) indicate that the placenta and its vascularity play a critical role in cardiac embryogenesis and CHD development. Indeed disorders such as maternal hypertension which can affect placental morphology and vascularity have been shown on meta-analysis to significantly increase the risk of CHD in the offspring of such pregnancies (Ramakrishnan et al. 2015; van Gelder et al. 2015). The placenta is an organ that is available in abundance for postnatal analysis.

In DNA methylation a single carbon atom or so-called ‘methyl group’ is transferred to and covalently bound to position #5 of the cytosine nucleotide ring of the cytosine-guanine (‘C-G’ or ‘CpG’) dinucleotide. This process converts the cytosine base to 5-methylcytosine (5mC). Classically, DNA methylation, particularly when it occurs in the promoter region of the gene which has a high number of CpG repeats, results in suppression of gene transcription or gene silencing. The 5mC can undergo further chemical modification to 5-hydroxymethylcytosine (5hmC). This hydroxymethylation was discovered to occur through the actions of a group of enzymes called ten-eleven translocation (TET) proteins. These are a group of three dioxygenases that catalyze the conversion of 5mC to 5hmC (Tahiliani et al. 2009).

Since 5hmC results from the conversion of 5mC, it is thought to be a mechanism for eliminating the former. The accumulation of 5hmC is linked to the regulation of gene transcription. Unlike 5mC, 5hmC binds much less avidly to gene repressor proteins such as the methyl-CpG-binding proteins such as MBD1, MBD2, and MBD4 (Jin S G, Kadam S, Pfeifer G P. Examination of the specificity of DNA methylation profiling techniques towards 5-methylcytosine and 5-hydroxymethylcytosine. Nucleic Acid Res 2010; 38:e125). Thus, beyond the measurement of 5mC concentration simultaneous information on 5hmC would provide more detailed epigenetic information and more precise information on gene transcription. 5hmC levels were found to be enhanced in the gene bodies of genes that are transcriptionally active (Nester et al. 2012) thus having an opposite effect on gene expression compared to 5mC.

Secondly, the concentration of 5hmC appears to be an important mechanism of tissue differentiation and a marker of tissue type (Nester et al. 2012). A major limitation of studies of cell-free fetal (cfF) DNA is the heavy contamination of the ‘fetal’ (more precisely placental DNA which is a fetal organ) with maternal DNA spilling from hemolyzed leukocytes. As a consequence, cfF DNA in the maternal circulation accounts for approximately 10-20% of cell-free (cf) DNA in pregnancy (Rafi et al. 2017). 5hmC levels are low in maternal leukocytes that that measuring cf fetal DNA 5hmC levels should improve the specificity of identifying fetal and/or placental cf DNA in maternal circulation.

### BRIEF DESCRIPTION OF THE DRAWINGS

FIGS. 1A and 1B show a heatmap generated using individual β-values for the significantly differentially methylated markers between cases and controls. Hierarchical cluster analysis shows clear separation of cases and controls based on differential CpG methylation. The CpG marker IDs are provided in the right column.

FIG. 2 shows the network of genes that are differentially methylated play important roles in cardiac embryonic development and congenital heart defect formation.

### SUMMARY

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter.

Foundational to Precision Medicine is the integration of Artificial Intelligence (AI), big data, omics technology, and the development of blood tests, to elucidate the pathogenesis of and accurately detect complex disorders (Mesko B, 2017). Epigenomic analysis of cell-free fetal DNA (cfF DNA) in combination with AI techniques was used to interrogate the pathogenesis of and to detect fetal non-syndromic CHD. In this prospective study, cfF DNA was extracted from maternal blood and whole-genome DNA methylation analysis was performed using the Illumina Infinium MethylationEPIC BeadChip arrays. Six different AI platforms including Deep Learning (DL), the most recent AI approach, were used for CHD detection. Ingenuity Pathway Analysis based on gene loci that were significantly differentially methylated, was used to investigate the molecular basis of CHD.

There were a total of 12 cases of isolated CHD and 26 controls. AI accurately detected CHD. Using RF, the combination of five CpG markers had an AUC AUC (95% CI)=0.98 (0.83−1) with 97% sensitivity and 93% specificity followed by SVM model with AUC (95% CI)=0.97 (0.87−1) with 98% sensitivity and 94% specificity for CHD detection. Logistic regression with cross-validation using CpG markers and a prior history of a CHD fetus had an AUC (95% CI)=0.98 (0.98−0.97) with 100% sensitivity and 85% specificity for CHD. Epigenetic dysregulation of genes and gene pathways involved in cardiogenesis and cardiac anomaly development were found in non-syndromic CHD. This provides biological confirmation or plausibility that the findings were not random occurrences.

The method described herein accurately detects fetal CHD using AI analysis of cfF DNA in maternal blood. Applicant's data provide further evidence of the role of epigenetic dysfunction in CHD non-syndromic development. Applicant's findings represent further progress in the development of fetal cardiovascular precision medicine.

### DETAILED DESCRIPTION

Birth defect, i.e. abnormalities developing in fetal life and present at birth, is the major cause of infant death, defined as death within a year of birth, in the USA. CHDs occur with a frequency of 8-9 cases per 1,000 live births. CHD is the most common group of severe birth defects and is the costliest in terms of hospitalization. Up to 25% of cases with major CHD in newborns are not diagnosed prior to discharge from the hospital.

Heart development in embryonic and fetal life requires the coordination and orchestration of a large number of different genes. A relatively small percentage of CHD cases is known to be related to gene mutations which are changes in the normal sequence in which the basic building block (“nucleotides”) are arranged in the DNA of the gene. Such mutations lead to malfunctioning or non-functioning of genes (i.e. altered amounts, of or the production of abnormal types of proteins) that are important for normal heart development.

In the last six decades, an important mechanism for controlling gene function called “epigenetics” has been discovered and extensively investigated. The term “epigenetics” can be used to describe the interaction between genes and the environment. These interactions do not result in changes to the genome sequence itself (no nucleotide sequence changes) but changes gene expression which still account for variations in phenotypic expression. Epigenetics is defined as heritable (i.e. passed onto offspring) changes in gene expression of cells that are not primarily due to mutations or changes in the sequence of nucleotides (adenine, thiamine, guanine, and cytosine) in the genes. Epigenetics is a reversible regulation of gene expression by several potential mechanisms. One such mechanism which is the most extensively studied is DNA methylation. Other mechanisms include changes in the 3-dimensional structure of the DNA, histone protein modification, and micro-RNA inhibitory activity. The epigenetic mechanisms are known to be extensively inter-related.

Cytosine refers to one of a group of four building blocks “nucleotides” from which DNA is constructed. The chemical structure of cytosine is in the form of a pyrimidine ring. Apart from cytosine, the other nucleotides or building blocks found in DNA are thiamine, adenine, and guanosine.

The term methylation refers to the enzymatic addition of a “methyl group” or single carbon atom to position #5 of the pyrimidine ring of cytosine which leads to the conversion of cytosine to 5-methyl-cytosine. The methylation of cytosine as described is accomplished by the actions of a family of enzymes named DNA methyltransferases (DNMT's). The 5-methyl-cytosine when formed is prone to mutation or the chemical transformation of the original cytosine to form thymine. Five-methyl-cytosines account for about 1% of the nucleotide bases overall in the normal genome.

The term hypermethylation refers to increased frequency or percentage methylation at a particular cytosine locus when specimens from an individual or group of interest are compared to a normal or control group.

Cytosine is usually paired with guanosine another nucleotide in a linear sequence along the single DNA strand to form CpG pairs. “CpG” refers to a cytosine-phosphate-guanosine chemical bond in which phosphate binds the two nucleotides together. In mammals, in approximately 70-80% of these CpG pairs the cytosine is methylated (Chatterjee R, Vinson C. Biochemica et Biophisica Acta 2012; 1819:763-70). The term “CpG island” refers to regions in the genome with a high concentration of CG dinucleotide pairs or CpG sites. “CpG islands” are often found close to genes in mammalian DNA. The length of DNA occupied by the CpG island is usually 300-3000 base pairs. The CG cluster is on the same single strand of DNA. The CpG island is defined by various criteria including i) the length of recurrent CG dinucleotide pairs occupying at least 200 bp of DNA and ii) a CG content of the segment of at least 50% along with the fact that the observed/expected CpG ratio should be greater than 60%. In humans, about 70% of the promoter regions of genes have high CG content. The CG dinucleotide pairs may exist elsewhere in the gene or outside of a gene and not know to be associated with a particular gene.

Approximately 40% of the promoter region (region of the gene which controls its transcription or activation) of mammalian genes has associated CpG islands and three-quarters of these promoter-regions have high CpG concentrations. Overall in most CpG sites scattered throughout the DNA, the cytosine nucleotide is methylated. In contrast, in the CpG sites located in the CpG islands of promoter regions of genes, the cytosine is unmethylated suggesting a role of the methylation status of cytosine in CpG Islands in gene transcriptional activity.

The methylation of cytosines associated with or located in a gene is classically associated with suppression of gene transcription. In some genes, however, increased methylation has the opposite effect and results in activation or increased transcription of a gene. One potential mechanism explaining the latter phenomenon is that methylation of cytosine could potentially inhibit the binding of gene suppressor elements thus releasing the gene from inhibition. Epigenetic modification, including DNA methylation, is the mechanism by which cells that contain identical DNA and genes experience the activation of different genes and result in the differentiation into unique tissues e.g. heart or intestines.

The present disclosure describes the use of epigenomic and Artificial Intelligence analytic techniques for accurate diagnosis or prediction of CHD, including CHD of prenatal and/or pediatric subjects, based on detecting cytosine methylation of nucleic acids of subjects. Methylation profiling was performed using Illumina Infinium arrays with over 850 k methylation markers according to the manufacturer's instructions. Methylation levels of CpG sites across the genome were examined in 12 CHD cases and compared to 26 of unaffected healthy matched controls. Pathway analysis was performed using Ingenuity pathway analysis to elucidate the mechanism of the disorder. In addition, the diagnostic accuracy of epigenomic markers for the detection of CHD was determined. The area under the receiver operating characteristics (AUC) curves and 95% CI and FDR p-values were calculated for the detection of CHD.

Several different Artificial Intelligence (AI) techniques including Deep Learning (DL), the newest form of AI, were used to predict CHD using i) epigenetic i.e. DNA methylation markers and ii) clinical and demographic markers.

In embodiments, the present disclosure describes a method for diagnosing CHD based on measurement of frequency or percentage methylation of cytosine nucleotides in various identified loci in a nucleic acid sample. In embodiments, the nucleic acid sample can be obtained from a biological sample of a patient in need thereof. The method includes obtaining a biological sample from a patient; extracting nucleic acid from the sample; assaying the sample to determine the percentage methylation of cytosine at loci throughout the genome; comparing the cytosine methylation level of the patient to a control; and calculating the individual risk of being diagnosed with CHD based on the cytosine methylation level at different sites throughout the genome. The control can be one or more characterized or known cases and/or a characterized or known group.

The methods described herein include obtaining nucleic acid from biological samples of a subject. The subject is an individual or a patient in need of (or in need there) diagnosis or is experiencing symptoms of CHD. The subject can also be undergoing routine screening. Examples of subjects include such as from an adult, a pediatric patient, an embryo, or a fetus. An “embryo” refers to the patient from the time of fertilization to the end of the eighth week of gestation. A “fetus” refers to the patient after the eighth week of gestation.

In embodiments, the patients could be adults and the control could be a well-characterized group of normal (healthy) people and/or a well-characterized population of CHD patients.

In embodiments, the patient could be a pediatric patient. The pediatric patient can be less than about 19 years old, about 15 to 19 years old, less than about 15 years old, about 10 to 15 years old, less than 10 years old (childhood), about 5 to 10 years old, less about 4 years old, about 1 to 4 years old, less than one-year-old (infant), or 28 days or less after birth (newborn or neonatal period). The patient can be an in utero patient, for example, an embryo or a fetus. When the patient is an embryo or fetus, the DNA can be obtained from a biological sample from the mother, the pregnant woman, carrying the embryo or fetus. The biological sample can be obtained from a pregnant woman in her first trimester, second trimester, or third trimester.

The control for pediatric patients could be a well-characterized group of normal (healthy) children of less than about 19 years old and/or a well-characterized population of CHD pediatric patients. Likewise, the control for the in utero patient could be a well-characterized group of normal (healthy) in utero patients and/or a well-characterized population of CHD in utero patients.

The well-characterized group of normal people (including adult, pediatric, and in utero patients) or CHD patients may include one or more normal people or CHD patients or may include a population of normal people or CHD patients.

The biological sample can be a body fluid such as blood, plasma, serum, urine, saliva, sputum, sweat, breath condensate, tears, genital secretion including cervical secretion, amniotic fluid, and umbilical cord blood obtained at birth. The biological sample can be a cervical swab for cell-free nucleic acid or exfoliated trophoblast cells, skin, hair, follicles/roots, and mucous membranes (cheek aka buccal scrapings or scrapings from the tongue). The biological sample can also include any internal body tissue of the patient, such as any tissue samples obtained from the patient including placental tissue from the newborn period or during fetal life. The placental tissue from an embryo or fetus can be obtained by placental biopsy or chorionic villus sampling (CVS). In embodiments, the biological sample can also include specimen from CVS. In embodiments, biological samples from a mother can include maternal blood, placenta, amniotic fluid, other body fluids during pregnancy, or other maternal body fluids.

Cells and nucleic acid from any biological samples which contain DNA can be used in the methods described herein for diagnosing and predicting CHD. Samples used for testing can be obtained from living or dead tissue and also archeological specimens containing cells or tissues.

The nucleic acid used in the method described herein can be obtained from cells. In embodiments, the nucleic acid includes fetal nucleic acid obtained directly from the fetus or the embryo, such as from the placenta or amniotic fluid by amniocentesis, or obtained from maternal body fluids or placental tissue, circulating fetal cells harvested from the maternal circulation, exfoliated placental cells, or cfF DNA in maternal circulation. In embodiments, the nucleic acid is obtained from amniotic fluid, fetal blood, or cord blood obtained during fetal life or at birth. In embodiments, the nucleic acid is DNA or RNA.

Since all cells, with few exceptions (mature red blood cells and mature platelets), contain nuclei and therefore DNA, the method described herein can be used to screen for CHD using DNA from any cells except for the two named above. Thus, any biological or tissue sample containing cells that contain nucleic acid such as DNA can be used in the method described herein. In addition, cell-free DNA released from cells that have been destroyed and which can be retrieved from body fluids can be used for such screening.

Cell-Free DNA (cf DNA). The nucleic acid can be DNA existing in the form of cf DNA. Cell-free DNA refers to DNA that has been released from cells as a result of natural cell death/turnover or as a result of disease processes. The cf DNA is released into the circulation and rapidly broken down into DNA fragment and can ultimately end up in other body fluids, such as urine. The techniques for the harvesting of cf DNA from the blood and other body fluids is well known in the arts (Li Y et al. Size separation of circulatory DNA in maternal plasma permits ready detection of fetal DNA polymorphisms. Clin Chem 2004; 50:1002-1011; Zimmerman B et al. Noninvasive prenatal aneuploidy testing of chromosomes 13, 18, 21, X, and Y, using targeted sequencing of polymorphic loci. Prenat Diagn 2012; 32:1233-41).

Cell-free DNA separation technique that was used coats and stabilizes maternal leukocytes preventing breakdown, release, and contamination from further maternally derived leukocyte DNA. To further enhance and target analysis of placental/fetal cf DNA rather than cf DNA originating from other maternal tissues, targeting techniques that can identify the tissue source of the cf DNA can be performed. An example includes the study of Lehmann-Werman R et al. (Lehmann-Werman et al. 2016). Using existing methylome databases they identified tissue-specific methylation patterns of cf DNA. Cell-free DNA was obtained from blood donors and based on the methylation compared this to the methylome databases and they were thus able to determine the tissue of origin of circulation cf DNA fragments e.g. pancreatic β-cells and oligodendrocytes from the brain.

5hmC level is tissue-specific, and beyond its reported correlation with gene expression levels. Beyond the gene expression information provided by 5hmC level in cf DNA, additional information of tissue origin of cf DNA opens up the ability to obtain DNA and epigenetic data from different organs based on a blood test. Studies (Nester et al. 2012), indicate that the level of 5hmC varies significantly between tissues. The levels of 5hmC are very low in the DNA of blood cells while comparatively high in placental tissue, and even higher in brain. Thus, measuring 5hmC in cf fetal DNA (of placental origin) would greatly redress the issue of contamination by maternal leucocyte cfF DNA that could be a concern when only 5mC levels are measured in cfF DNA. This is so because the levels of 5hmC in leukocytes are so low compared to cfF DNA derived from the placenta, the object of interest in this CHD proposal. Overall, therefore, the use of 5mC and 5hmC measurements of cf DNA targeted epigenomic analysis of placental tissue can be performed for the detection and to provide further enhanced mechanistic information on CHD and other pregnancy disorders in which the placenta is affected.

Methylation Assays. Several quantitative methylation assays are available. These include COBRA™ which uses methylation sensitive restriction endonuclease, gel electrophoresis and detection based on labeled hybridization probes. Another available technique is the Methylation Specific PCR (MSP) for amplification of DNA segments of interest. This is performed after sodium ‘bisulfite’ conversion of cytosine using methylation sensitive probes. MethyLight™, a quantitative methylation assay-based uses fluorescence based PCR. Another method used is the Quantitative Methylation (QM™) assay, which combines PCR amplification with fluorescent probes designed to bind to putative methylation sites. Ms-SNuPE™ is a quantitative technique for determining differences in methylation levels in CpG sites. As with other techniques, bisulfite treatment is first performed leading to the conversion of unmethylated cytosine to uracil while methylcytosine is unaffected. PCR primers specific for bisulfite converted DNA is used to amplify the target sequence of interest. The amplified PCR product is isolated and used to quantitate the methylation status of the CpG site of interest. The preferred method of measurement of cytosine methylation is the Illumina method.

Illumina Method. For DNA methylation assay the Illumina Infinium® Human Methylation 450 Beadchip or Illumina Infinium MethylationEPIC BeadChip assay can be used used for quantitative methylation profiling. Briefly nucleic acid, for example, genomic DNA, is obtained. Using techniques widely known in the trade, the nucleic acid is isolated using commercial kits. Proteins and other contaminants were removed from the nucleic acid using proteinase K. The nucleic acid is removed from the solution using available methods such as organic extraction, salting out, or binding the DNA to a solid phase support.

Methylation Analysis-Illumina's Infinium Human Methylation 450 Bead Chip system or Ilumina Infinium MethylationEPIC BeadCHip arrays can be used for genome-wide methylation analysis. Nucleic acid, such as DNA, (500 ng) is subjected to bisulfite conversion to deaminate unmethylated cytosines to uracil with the EZDNA Methylation Gold kit or EZ-96 Methylation Kit (Zymo Research) using the standard protocol for the Infinium assay. The DNA is enzymatically fragmented and hybridized to the Illumina BeadChips. BeadChips contain locus-specific oligomers and are in pairs, one specific for the methylated cytosine locus and the other for the unmethylated locus. A single base extension is performed to incorporate a biotin-labeled ddNTP. After fluorescent staining and washing, the BeadChip is scanned and the methylation status of each locus is determined using BeadStudio software (Illumina). Experimental quality was assessed using the Controls Dashboard that has sample-dependent and sample-independent controls target removal, staining, hybridization, extension, bisulfite conversion, specificity, negative control, and non-polymorphic control. The methylation status is the ratio of the methylated probe signal relative to the sum of methylated and unmethylated probes. The resulting ratio indicates whether a locus is unmethylated (0) or fully methylated. Differentially methylated sites are determined using the Illumina Custom Model and filtered according to p-value using 0.05 as a cutoff.

Bisulfite Conversion. As described in the Infinium® Assay Methylation Protocol Guide, nucleic acid is treated with sodium bisulfite which converts unmethylated cytosine to uracil, while the methylated cytosine remains unchanged. The bisulfite converted nucleic acid is then denatured and neutralized. The denatured nucleic acid is then amplified. Bisulfite based analysis, the current technique for differentiating methylated from unmethylated cytosine, does not distinguish 5mC from 5hmC. New techniques include but not limited to thin-layer chromatography assay (Kriaucionis et al. 2009), and chemical tagging of 5hmC (Song et al. 2011), immunoprecipation (Nester et al., 2012), and more recently commercially available 5hmC whole exome and even whole-genome sequencing techniques can be used to provide detailed information on epigenetic changes in cfF DNA.

In embodiments, using the Illumina Infinium Assays for whole-genome (using genomic DNA) methylation studies, significant differences in the frequency (level or percentage) of methylation of specific cytosine nucleotides associated with particular genes were demonstrated in the CHD group when compared to a normal group. The differences in cytosine methylation levels are highly significant and of sufficient magnitude to accurately distinguish the CHD from the normal group. Thus, the methods described herein can be used as a test to screen for CHD cases among a mixed population with CHD and normal cases.

The whole-genome application process increases the amount of DNA by up to several thousand-fold. The next step uses enzymatic means to fragment the DNA. The fragmented DNA is next precipitated using isopropanol and separated by centrifugation. The separated DNA is next suspended in a hybridization buffer. The fragmented DNA is then hybridized to beads that have been covalently limited to 50mer nucleotide segments at a locus-specific to the cytosine nucleotide of interest in the genome. There is a total of over 500,000 bead types specifically designed to anneal to the locus where the particular cytosine is located. The beads are bound to silicon-based arrays. There are two bead types designed for each locus, one bead type represents a probe that is designed to match to the methylated locus at which the cytosine nucleotide will remain unchanged. The other bead type corresponds to an initially unmethylated cytosine which after bisulfite treatment is converted to a thiamine nucleotide. Unhybridized (not annealed to the beads) DNA is washed away leaving only DNA segments bound to the appropriate bead and containing the cytosine of interest. The bead-bound oligomer, after annealing to the corresponding patient DNA sequence, then undergoes single base extension with fluorescently labeled nucleotide using the ‘overhang’ beyond the cytosine of interest in the patient DNA sequence as the template for extension.

If the cytosine of interest is unmethylated then it will match perfectly with the unmethylated or “U” bead probe. This enables single base extensions with fluorescent labeled nucleotide probes and generates fluorescent signals for that bead probe that can be read in an automated fashion. If the cytosine is methylated, single base mismatch will occur with the “U” bead probe oligomer. No further nucleotide extension on the bead oligomer occurs however thus preventing incorporation of the fluorescent tagged nucleotides on the bead. This will lead to low fluorescent signal form the bead “U” bead. The reverse will happen on the “M” or methylated bead probe.

Laser is used to stimulate the fluorophore bound to the single base used for the sequence extension. The level of methylation at each cytosine locus is determined by the intensity of the fluorescence from the methylated compared to the unmethylated bead. Cytosine methylation level is expressed as “β” which is the ratio of the methylated bead probe signal to total signal intensity at that cytosine locus. These techniques for determining cytosine methylation have been previously described and are widely available for commercial use.

The present disclosure describes the use of a commercially available methylation technique to cover up to 99% Ref Seq genes involving approximately 16,000 genes and 450,000 cytosine nucleotides down to the single nucleotide level, throughout the genome (Infinium Human Methylation 450 Beach Chip Kit or Infinium MethylationEPIC BeadChip). The frequency of cytosine methylation at single nucleotides in a group of CHD cases compared to controls is used to estimate the risk or probability of being diagnosed with CHD. The cytosine nucleotides analyzed using this technique included cytosines within CpG islands and those at further distances outside of the CpG islands i.e. located in “CpG shores” and “CpG shelves” and even more distantly located from the island so called “CpG seas”.

The cytosine evaluated as described herein includes but are not limited to cytosines in CpG islands located in the promoter regions of the genes. Other areas targeted and measured include the so called CpG island ‘shores’ located up to 2000 base pairs distant from CpG islands and ‘shelves’ which is the designation for DNA regions flanking shores. Even more distant areas from the CpG islands so called “seas” were analyzed for cytosine methylation differences. The extragenic cytosine loci, located outside of known genes (however they could potentially maintain long-distance control of unspecified genes) also detected CHD with moderate, good and excellent accuracy as indicated.

Identification of Specific Cytosine Nucleotides. Reliable identification of specific cytosine loci distributed throughout the genome has been detailed (Illumnia) in the document: “CpG Loci Identification. A guide to Illumina's method for unambiguous CpG loci identification and tracking for the GoldenGate® and Infinium™ assays for Methylation.” A brief summary follows. Illumina has developed a unique CpG locus identifier that designates cytosine loci based on the actual or contextual sequence of nucleotides in which the cytosine is located. It uses a similar strategy as used by NCBI's re SNP IPS (rs #) and is based on the sequence flanking the cytosine of interest. Thus, a unique CpG locus cluster ID number is assigned to each of the cytosine undergoing evaluation. The system is reported to be consistent and will not be affected by changes in public databases and genome assemblies. Flanking sequences of 60 bases 5′ and 3′ to the CG locus (i.e. a total of 122 base sequences) are used to identify the locus. Thus, a unique “CpG cluster number” or cg # is assigned to the sequence of 122 bp which contains the CpG of interest. The cg # is based on Build 37 of the human genome (NCB137). Accordingly, only if the 122 bp in the CpG cluster is identical is there a risk of a locus being assigned the same number and being located in more than one position in the genome. Three separate criteria are utilized to track individual CpG locus based on this unique ID system. Chromosome number, genomic coordinate and genome build. The lesser of the two coordinates “C” or “G” in CpG is used in the unique CG loci identification. The CG locus is also designated in relation to the first ‘unambiguous” pair of nucleotides containing either an ‘A’ (adenine) to ‘T’ (thiamine). If one of these nucleotides is 5′ to the CG then the arrangement is designated TOP and if such a nucleotide is 3′ it is designate BOT.

In addition, the forward or reverse DNA strand is indicated as being the location of the cytosine being evaluated. The assumption is made that methylation status of cytosine bases within the specific chromosome region is synchronized.

Cytosine Methylation for the diagnosing CHD Using ROC Curve. To determine the accuracy of the methylation level of a particular cytosine locus for CHD prediction, different threshold levels of methylation e.g. 10%, ≥20%, ≥30%, ≥40% etc. at the site was used to calculate sensitivity and specificity for CHD diagnosis. Thus, for example using 0% methylation at a particular cg locus, cases with methylation levels above this threshold would be considered to have a positive test and those with lower than this threshold are interpreted as a negative methylation test. The percentage of CHD cases with a positive test in this example, 10% methylation at this particular cytosine locus, would be equal to the sensitivity of the test. The percentage of normal (non-CHD) cases with cytosine methylation levels of ≤10% at this locus would be considered the specificity of the test. False positive rate is here defined as the number of normal cases with a (falsely) abnormal test result and sensitivity is defined as the number of CHD cases with (correctly) abnormal test result e.g. the level of methylation 10% at this particular cg location. A series of threshold methylation values are evaluated e.g. ≥ 1/10, ≥ 1/20, ≥ 1/30 etc., and used to generate a series of paired sensitivity and false positive values for each locus. A receiver operating characteristic (ROC) curve which is a plot of data points with sensitivity values on the Y-axis and false positivity rate on the X-axis is generated. This approach can be used to generate ROC curves for each individual cytosine locus that displays significant methylation differences between cases and CHD groups. In this instance the computer program ROCR package-version 3.4 ((https://CRAN.R-project.org/package=ROCR) was used to generate the area under the ROC curves.

The ROC curve is a graph plotting sensitivity—defined in this setting as the percentage of CHD cases with a positive test or abnormal cytosine methylation levels at a particular cytosine locus on the Y axis and false positive rate (1-specificity or 100%—specificity, when the latter is expressed as a percentage)—i.e. the number of normal (non-CHD) cases with abnormal cytosine methylation at the same locus—on the X-axis. Specificity is defined as the percentage of normal (non-CHD) cases with normal methylation levels at the locus of interest or a negative test. False positive rate refers to the percentage of normal individuals falsely found to have a positive test (i.e. abnormal methylation levels); it can be calculated as 100-specificity (%) or expressed as a decimal format [1-specificity (expressed as a decimal point)].

The area under the ROC curves (AUC) indicates the accuracy of the test in identifying normal from abnormal cases. The AUC is the area under the ROC plot from the curve to the diagonal line from the point of intersection of the X- and Y-axes and with an angle of incline of 45°. The higher the area under ROC curve the greater is the accuracy of the test in predicting the condition of interest. An area under the ROC=1.0 indicates a perfect test, which is positive (abnormal) in all cases with the disorder and negative in all normal cases (without the disorder). Methylation assay refers to an assay, a large number of which are commercially available, for determining the level of methylation at a particular cytosine in the genome. In this particular context, this approach can be used to distinguish the level of methylation in affected cases (CHD) compared to unaffected controls.

Logistic regression analysis can be used for calculation of sensitivity and specificity for the prediction of CHD based on methylation of cytosine loci.

Standard statistical testing using p-values to express the probability that the observed difference between cytosine methylation at a given locus between CHD and control DNA specimens can be performed. More stringent testing of statistical significance using False Discovery Rate (FDR) for multiple comparisons was also performed. The FDR gives the probability that positive results were due to chance when multiple hypothesis testing is performed using multiple comparisons.

Statistical Analyses. The present disclosure describes a method for predicting, diagnosing, detecting CHD in a subject, and/or calculating the risk of the subject in being diagnosed with CHD or even a particular type of CHD. This calculation can be based on logistic regression analysis leading to the identification of the significant independent predictors among a number of possible predictors (e.g. methylation loci) known to be associated with CHD or increased risk of being diagnosed with CHD. Cytosine methylation levels at different loci can be used by themselves or in combination with other known risk predictors for CHD, such as prenatal exposure to toxins—“yes” or “no” (e.g. alcohol or maternal smoking, maternal diabetes, family history combined with methylation levels in a single or multiple loci) which are known to be associated with increased risk of CHD as described in this application. For example, the probability of an individual being affected can be derived from the probability equation based on the logistic regression:

P_{CHD}=1/1+e^{(B1×1+B2×2+B3×3 . . . Bn×n)}

where ‘x’ refers to the magnitude or quantity of the particular predictor (e.g. methylation level at a particular locus) and “β” or β-coefficient refers to the magnitude of change in the probability of the outcome (e.g., CHD) for each unit change in the level of the particular predictor (x), the β values are derived from the results of the logistic regression analysis. These β values would be derived from multivariable logistic regression analysis in a large population of affected and unaffected individuals. Values for x1, x2, x3, etc, representing in this instance methylation percentage at different cytosine locus would be derived from the individual being tested while the β-values would be derived from the logistic regression analysis of the large reference population of affected (CHD) and unaffected cases mentioned above. Based on these values, an individual's probability of having a type of CHD can be quantitatively estimated. Probability thresholds are used to define individuals at high risk (e.g. a probability of ≥ 1/100 of CHD may be used to define a high risk individual triggering further evaluation such as an one or more of the following: echocardiograms, pulse oximetry measurements at birth and the like, while individuals with risk < 1/100 would require no further follow-up. The threshold used will among other factors be based on the diagnostic sensitivity (number of CHD cases correctly identified), specificity (number of non-CHD cases correctly identified as normal), risk and cost of ECHOcardiogram and related interventions pursuant to the designation of an individual as “high risk” for CHD and such factors. Logistic regression analysis is well known as a method in disease screening for estimating an individual's risk for having a disorder. (Royston P, Thompson S G. Model-based screening by risk with application in Down's syndrome. Stat Med 1992; 11:257-68.)

Individual risk of CHD can also be calculated by using methylation percentages (reported as β-coefficients) at the individual discriminating cytosine locus by themselves or using different combinations of loci based on the method of overlapping Gaussian distribution or multivariate Gaussian distribution (Wald N J, Cuckle H S, Deusem J W et al (1988) Maternal serum screening for down syndrome in early pregnancy. BMJ 297, 883-887.) where the variable would be methylation level/percentage methylation at a particular (or multiple) loci so called. Alternatively if methylation percentages or β-coefficients are not normally distributed (i.e. non-Gaussian), normal Gaussian distribution would be achieved if necessary by logarithmic transformation of these percentages.

As an example, two Gaussian distribution curves are derived for methylation at particular loci in the CHD group and the normal populations. Mean, standard deviation and the degree of overlap between the two curves are then calculated. The ratio of the heights of the distribution curves at a given level of methylation will give the likelihood ratio or factor by which the risk of having CHD is increased (or decreased) at a particular level of methylation at a given locus. The likelihood ratio (LR) value can be multiplied by the background risk of CHD in the general population and thus give an individual's risk of CHD based on methylation level at the CG site(s) chosen. Information on the background population risk of CHD in the newborn population is available from several sources (one such example is Hoffman J L et al Am Heart J 2004; 147:425-439). Similar information is available for prenatal and later postnatal life.

Artificial Intelligence (AI). One or more AI algorithms can be used in combination with the methods described herein to improve the accuracy for predicting and/or diagnosing CHD. Representative examples of AI algorithms include Random Forest (RF), Support Vector Machine (SVM), Linear Discriminant Analysis (LDA), Prediction of Analysis for Microarrays (PAM), Generalized Linear Model (GLM), and deep learning (DL).

Random Forest (RF) is a supervised classification algorithm used for regression, classification and other tasks. Multiple decision tree predictive models are randomly generated in the training phase and the mode of the classes and mean prediction of the individual trees are generated as outputs. There is a direct relationship between the number of trees in the forest and the results it can get: the larger the number of trees, the more accurate the result. The difference between Random Forest algorithm and the decision tree algorithm is that in Random Forest, the processes of finding the root node and splitting the feature nodes will run randomly. The decision tree is a decision support tool that uses a tree-like graph to show the possible consequences. If one inputs a training dataset with targets and features into the decision tree, it will formulate a set of rules. Overfitting is one critical problem that may make the results worse in decision trees, but for Random Forest algorithm, if there are enough trees in the forest, the classifier won't overfit the model. Another advantage is the classifier of Random Forest can handle missing values, and the last advantage is that the Random Forest classifier can be modeled for categorical values.

Support vector machine (SVM) is primarily a classifier method that performs classification tasks by constructing hyperplanes in a multidimensional space that separates cases of different class labels. SVM supports both regression and classification tasks and can handle multiple continuous and categorical variables. Suppose some given data points each belong to one of two classes, and the goal is to decide which class a new data point will be in. In the case of support vector machines, a data point is viewed as a p-dimensional vector (a list of p numbers), and we want to know whether we can separate such points with a (p−1)-dimensional hyperplane. This is called a linear classifier. There are many hyperplanes that might classify the data. One reasonable choice as the best hyperplane is the one that represents the largest separation, or margin, between the two classes. We choose the hyperplane so that the distance from it to the nearest data point on each side is maximized. If such a hyperplane exists, it is known as the maximum-margin hyperplane and the linear classifier it defines is known as a maximum margin classifier; or equivalently, the perceptron of optimal stability.

Linear Discriminant Analysis (LDA) is a classification method originally developed in 1936 by R. A. Fisher. It is simple, mathematically robust, and often produces models whose accuracy is as good as more complex methods. LDA is based upon the concept of searching for a linear combination of variables (predictors) that best separates two classes (targets). It is closely related to analysis of variance (ANOVA) and regression analysis, which also attempt to express one dependent variable as a linear combination of other features or measurements.

Prediction Analysis for Microarrays (PAM) is a statistical technique for class prediction from gene expression data using the nearest shrunken centroids. This method identifies the subsets of genes that best characterize each class.

Generalized Linear Models (GLMs) are a broad class of models that include, for example, linear regression, ANOVA, Poisson regression, log-linear models, but there are some limitations of GLMs, such as, linear function, e.g. can have only a linear predictor in the systematic component, and responses must be independent.

Generally, classical machine learning techniques make predictions directly from a set of features that have been pre-specified by the user. However, representation learning techniques transform features into some intermediate representation prior to mapping them to final predictions. Deep Learning (DL) is a form of representation learning that uses multiple transformation steps to create very complex features. DL is widely applied in pattern recognition, image processing, computer vision, and recently in bioinformatics. DL is categorized into feed-forward artificial neural networks (ANNs), which uses more than one hidden layer (y) that connects the input (x) and output layer (z) via a weight (VV) matrix. The weight matrix W which is expected to minimize the difference between the input layer (x) and the output layer (z) is considered as the best one and chosen by the system to get the best results.

Types of CHDs and Prevention and Treatment of CHDs. The methods described herein can be used to diagnose, detect, or predict one or more hearts defects in a subject. There are various types of heart defects which are often grouped by the part of the heart that is affected. The defects could be of the heart valves, the septum, the interior valves of the heart, or the ventricles. Valve defects include for example defects of the aortic and pulmonary valves. Defects in the septum include problems with the wall that separates the right hand and left hand chambers of the heart. When the septum fails to fully close during development of the heart, small holes results in the wall separating the right and left hand chambers. Some defects are due to valves that separate the top and bottom chambers of the heart failing to form properly. Some types of heart defects include a combination of different defects or have to do with the way the heart looped while it was developed.

Examples of some CHDs include aortic valve stenosis (AVS), hypoplastic left heart syndrome (HLHS), ventricular septal defect (VSD) including VSD with atrial septal defect, Tetralogy of Fallot (TOF), coarctation of the aorta (Coarct), atrial septal defect (ASD), pulmonary stenosis (PS) including pulmonary valve stenosis and pulmonary artery valve stenosis, pulmonary artery altresia including those with pulmonary valve stenosis, truncus arteriosus, double aortic arch, and biscuspid A-V valve including those with dilated main pulmonary artery. Patients diagnosed with one or more of these types of CHDs require surgery to prevent severe complications or death.

The methods described herein provide accurate and early detection and prediction of one or more heart defects. Early prenatal diagnosis of CHD allows early evaluation and optimal treatment at birth and reduces the death rate (Holland B J et al. Ultrasound Ostet. Genecology 2015: 45:631-638) and morbidities compared to cases in which the diagnosis is made later after birth. In addition, early postnatal diagnosis of CHD is associated with improved survival compared with late diagnosis in critical CHD cases (Eckersley L, Sadler L etl. Arch Dis Child 2015: 0:1-5). Thus, the methods described herein promote accurate prenatal diagnosis which facilitates earlier evaluation, for example during the newborn period, treatment, and improved survival rate in CHD cases.

The cytosine methylation described herein refers to the chemical addition of a methyl or single carbon atom to the cytosine nucleotide. An important dietary source of the carbon atom used in cytosine methylation is folic acid. Given Applicant's findings demonstrating the importance of cytosine methylation in the pathogenesis of CHD, it is reasonable to expect that dietary folic acid supplementation would reduce the risk of CHD. Currently, folic acid fortification in grains and bread, and direct supplementation for consumption by the entire population including pregnant women has been a standard of care for the prevention of neural tube defects. This presents a ‘natural’ experiment by which to judge the value of folate supplementation for the prevention of CHD. Studies in China (Mao B. et al. 2017), the U.S.A. (Botto et al. 2003), Europe (Czeizel et al. 1998), and Canada (Ionescu-Ittu et al. 2009) showed a beneficial effect of folic acid supplementation in reducing CHD. It should be noted that a significant percentage of pregnant women might not respond adequately to folate supplementation. This is because a significant percentage of females in some populations has a mutation in the methylenetetrahydrofolate reductase (MTHFR) gene which codes for the similarly named enzyme responsible for folate metabolism the end result of which is the generation of methyl carbon involved in cytosine methylation. The result of MTHFR is therefore to impair cytosine methylation. The frequency of this mutation can be as high as 20% in some populations. Also, when the mutation is present enzyme activity is reduced by as much as 30% (Botto et al. 2000). Thus, MTHFR mutation would be expected to blunt the effectiveness of folic acid supplementation in such populations.

Alternate sources of methyl group that are unaffected by the MTHFR gene mutation fortunately exist. These include choline and betaine and exist in dietary sources such as broccoli, spinach, beets, liver, and other foods. Based on Applicant's findings of the importance of DNA methylation in CHD, population fortification and individual supplementation programs for choline and betaine could be evaluated. Further, current evidence indicates that less than one tenth of the U.S.A. population including pregnant women has adequate choline consumption (Zeisel et al. 2009). The risk of deficiency in pregnancy is amplified by the fact that this is a period of increased choline demand.

Laboratory evidence exists for the importance of betaine, an alternate source of single carbon for cytosine methylation, for the prevention of CHD (Karunamuni et al. 2017). Prenatal alcohol exposure is a significant risk factor for CHD. It has been estimated that 10% of pregnant women in the U.S.A. drink alcohol and that 3% have high levels of consumption (Tan et al. 2015) Prenatal alcohol intake has been shown to interfere with single carbon metabolism and DNA methylation in mice (Liu et al. 2009). In the study by Karunamuni et al. (Karunamuni et al. 2017) supplementation with betaine in pregnant mice exposed to alcohol reduced the frequency of CHD with beneficial effects on the structural changes to the heart and great vessels that result from alcohol exposure. Importantly, alcohol caused a reduction in the 5mC nucleotide levels in cardiac neural crest cells of the pups affected with CHD. Cardiac neural crest cells are a specialized group of cells that are embryologically critical for the development of important heart structures such as the interventricular septum, the aorta, pulmonary artery, and other vessels. The 5mC levels of these cardiac neural crest cells were returned to normal with betaine supplementation of pregnant mice who were exposed to prenatal alcohol. The preceding information confirms the importance of methylation changes in heart development. However, they do not provide any information on the use of methylation levels in cf DNA for CHD detection. The method described herein is a minimally invasive blood test to evaluate the mechanisms of CHD. The test can be performed closer to the early development of the heart in the first trimester. Cell-free fetal DNA is known to be deported into the maternal circulation from early in the first trimester. Knowledge of the methylation status of a developing fetus can potentially serve as a basis for supplementation with folate, choline, or betaine in individual patients to help prevent or mitigate the development or severity of CHD.

From a population perspective, the knowledge generated from Applicant's findings i.e. use of cfF DNA from a developing fetus showing evidence of profound changes in cytosine methylation in important cardiac genes in CHD cases could form the basis of a policy of population supplementation with choline and betaine. This would be particularly important for populations with a high rate of MTHFR mutation which renders folate supplementation less effective. Thus, Applicant's findings can be used as a basis for important prophylactic and even therapeutic intervention.

Once a patient is diagnosed with CHD, medication can be used, to keep the heartbeat regular. Immediate treatment of CHD patients includes providing sufficient oxygen level to the patient until repair to the heart can be performed. Surgery, such as open-heart surgery, can be performed on patients to repair any defect to the heart. Other methods of treatment depending on the CHD include antibiotics, cardiac catheterization procedures, open-heart surgery, and heart transplant. Medications for CHD can include angiotensin-converting enzyme (ACE) inhibitors, angiotensin II receptor blockers (ARBs), beta blockers, diuretics, antihypertensives, and others.

In embodiments, the methods described herein enables prophylaxis against development of CHD in a future pregnancy. As an example, the pregnant woman can be supplemented with folic acid or folates.

Tables of Genes and Genomic Loci (CHD Markers). The present disclosure reports a strong association between cytosine methylation status at a large number of cytosine sites throughout the genome using stringent False Discover Rate (FDR) analysis with q-values <0.05 and with many q-values as low as <1×10⁻³⁰ depending on particular cytosine locus being considered. A total of 12 cases of CHD and 26 normal controls underwent epigenomic analysis. Significant differences in cytosine methylation patterns at multiple loci throughout the DNA that was found in all CHD cases tested compared to normal. The genomic loci described herein are located in or related to known genes. These findings are consistent with the altered expression of multiple genes in CHD cases compared to controls.

Tables 1 to 6 provide genomic loci that can be used individually to predict, detect, or diagnose CHD in patients. The genomic loci are provided underneath each table of Tables 2 to 6. One or more of the genomic loci in Tables 1 to 6 can be selected for predicting or diagnosing CHD in patients. In embodiments, two or more of the loci can be selected from the loci in Table 1, 2, 3, 4, 5, and/or 6.

A total of 5918 CpG genomic loci encompassing 4976 genes (FDR p-value ≤0.01) were identified. Table 1 provides the top 1000 genomic loci obtained by genome-wide methylation profiling performed using cfF DNA. One or more, two or more, up to and including all 1000 of the genomic loci in Table 1 can be selected for predicting, detecting, or diagnosing CHD in a patient. In embodiments, one or more, two or more, up to and including 5, 10, 15, 20, 30, 40, 50, 60, 70, 80, 90, 100, 200, 300, 400, 500, 600, 700, 800, 900, or 1000 genomic loci disclosed in Table 1 can be selected for predicting CHD. In embodiments, the genomic loci have an AUC (with 95% CI), ≥0.70, 0.75, 0.80 0.85, 0.86, 0.87, 0.88, 0.89, 0.90, 0.91, 0.92, 0.93, or 0.94. Of the 1000 genomic loci, 130 CpGs were hypomethylated, and 870 markers were hypermethylated in association with CHD. 53 hypomethylated and 486 hypermethylated markers were found to be differentially methylated by ≥10% methylation difference. The three genomic loci that were above 20% of methylation difference are, cg06301252 (PTPRN2) with 33.62%, cg02807450 (MTMR2) with −21.15%, and cg12900404 (DOCK10) with −20.13%. 126 CpG loci showed AUC ≥0.80 individually, indicating very good to excellent predictive accuracy for the disease prediction. In embodiments, the genomic loci for detecting, predicting, and/or diagnosing CHD include cg06301252, cg02807450, and cg12900404.

AUC integrates sensitivity and specificity values and gives a more precise indication of the accuracy of the test. AUC (with 95% CI) indicates an AUC with a statistically significant 95% confidence interval. An AUC ≥0.70 indicates a clinically useful test.

Tables 2-6 provide the genomic loci obtained by AI analysis using 6 different platforms including SVM, GLM, PAM, RF, LDA, and DL. Although the loci are provided under each of the tables, they belong to the numbered table summarizing the AUC, sensitivity, and specificity for each algorithm. In embodiments, the genomic loci for predicting, detecting, and diagnosing includes the one to five loci for each of the 6 algorithms listed in Tables 2-6. The top 5 predictive markers for each model are provided in each table in a descending order (under each table).

In embodiments, the genomic loci are selected from the algorithms having an AUC (with 95% CI), ≥0.8800, 0.8900, 0.9000, 0.9100, 0.9200, 0.9300, 0.9400, 0.9500, 0.9600, or 0.9700. In embodiments, the genomic loci are selected from algorithms with a sensitivity and/or specificity of ≥0.8700, 0.8800, 0.8900, 0.9000, or 0.9100. In embodiments, the genomic loci are selected from the one to five loci of RF, SVM, or DL of Table 2.

Table 3 shows comparable predictive performance was achieved when demographic markers were considered along with CpG loci. Table 4 shows that high performance was achieved when only markers meeting stringent GWAS thresholds were considered. Table 5 shows high performance was achieved when demographics markers and CpG loci meeting stringent GWAS thresholds were considered. Table 6 shows that when only markers showing high level of methylation change, for example 1.5 fold or greater, are used high predictive accuracies are seen. Table 6 shows that when only markers showing high level of methylation change, for example 1.5 fold or greater, are used high predictive accuracies are seen.

Ranges described throughout the application include the specified range, the sub-ranges within the specified range, the individual numbers within the range, and the endpoints of the range. For example, description of a range such as from one or more up to 1000 includes subranges such as from one or more to 500 or more, from 10 or more to 20 or more, from one or more to five or more, as well as individual numbers within that range, for example, 1, 2, 3, 4, 5, 10, 20, 100, and 500. Moreover, as a further example, the description of a range of ≥0.70 would include all the individual numbers from 0.70 to 1.00 and including 0.70 and 1.0.

The results presented herein confirm that based on the differences in the level of methylation of the cytosine sites between CHD and normal cases throughout the whole human genome, the predisposition to or risk of having a CHD can be determined.

The genomic loci reported enables targeted screening studies for the prediction and detection of CHD based on cytosine methylation throughout the genome. They also permit improved understanding of the mechanism of development of CHD for example by evaluating the cytosine methylation data using gene ontology analysis. In embodiments, the genomic loci are used in many different combinations to predict, detect, or diagnose CHD in a subject. In embodiments, the genomic loci are used to determining or calculating the risk or predisposition of a to having a CHD at any time prenatal or during any period of postnatal life of a subject.

TABLE 1

| | | | | | | | | % | % | % |
|---|---|---|---|---|---|---|---|---|---|---|
| | | | | | | | | Meth- | Meth- | Meth- |
| | | | | Fold | | | | ylation | ylation | ylation |
| TargetID | CHR | Gene | FDR p-Val | change | AUC | CI_lower | CI_upper | Cases | Control | Difference |
| cg04761177 | 16 | ATP2A1 | 2.40281E−12 | 0.90 | 0.94 | 0.86 | 1.00 | 79.73 | 88.97 | −9.24 |
| cg03790075 | 10 | SEMA4G | 1.28889E−13 | 1.09 | 0.90 | 0.81 | 1.00 | 90.76 | 83.61 | 7.15 |
| cg04626875 | 22 | SULT4A1 | 2.05295E−39 | 1.14 | 0.88 | 0.78 | 0.99 | 86.85 | 76.46 | 10.39 |
| cg13598434 | 17 | HEXIM1 | 1.76672E−12 | 0.55 | 0.88 | 0.78 | 0.99 | 12.20 | 22.18 | −9.99 |
| cg26389316 | 3 | SRGAP3 | 8.85912E−41 | 1.18 | 0.87 | 0.76 | 0.98 | 88.64 | 75.20 | 13.44 |
| cg06297857 | 16 | CSNK2A2 | 1.20609E−38 | 1.10 | 0.87 | 0.76 | 0.98 | 90.77 | 82.20 | 8.57 |
| cg23037321 | 1 | MR1 | 1.32216E−12 | 0.35 | 0.87 | 0.76 | 0.98 | 3.75 | 10.74 | −6.99 |
| cg24412848 | 12 | POLR3B | 1.10692E−19 | 0.32 | 0.87 | 0.75 | 0.98 | 4.47 | 13.88 | −9.41 |
| cg22258841 | 6 | ZSCAN12 | 3.19858E−42 | 1.27 | 0.87 | 0.75 | 0.98 | 78.18 | 61.73 | 16.45 |
| cg03184447 | 22 | DEPDC5 | 3.37066E−12 | 1.16 | 0.87 | 0.75 | 0.98 | 75.46 | 64.82 | 10.64 |
| cg18198743 | 8 | TMEM68 | 1.29901E−12 | 1.13 | 0.87 | 0.75 | 0.98 | 81.43 | 71.86 | 9.57 |
| cg01637563 | 7 | PON1 | 1.24725E−39 | 1.15 | 0.86 | 0.75 | 0.98 | 84.12 | 73.23 | 10.89 |
| cg02970663 | 2 | LRRFIP1 | 5.01498E−39 | 1.12 | 0.86 | 0.75 | 0.98 | 85.53 | 76.05 | 9.48 |
| cg16089031 | 7 | INTS1 | 2.26947E−43 | 1.51 | 0.86 | 0.74 | 0.98 | 55.42 | 36.72 | 18.70 |
| cg05279901 | 19 | ATP1A3 | 1.05679E−11 | 0.43 | 0.86 | 0.74 | 0.98 | 5.56 | 12.93 | −7.37 |
| cg04541077 | 16 | TAOK2 | 2.36884E−41 | 1.32 | 0.86 | 0.74 | 0.97 | 60.68 | 46.02 | 14.66 |
| cg22934935 | 6 | L3MBTL3 | 1.77656E−40 | 1.18 | 0.86 | 0.74 | 0.97 | 83.38 | 70.59 | 12.78 |
| cg21854952 | 17 | HIC1 | 5.75765E−14 | 1.90 | 0.86 | 0.74 | 0.97 | 21.99 | 11.57 | 10.42 |
| cg11387612 | 22 | EIF4ENIF1 | 8.49731E−39 | 1.11 | 0.86 | 0.74 | 0.97 | 87.33 | 78.40 | 8.94 |
| cg14017326 | 1 | SRGAP2 | 1.01264E−38 | 1.11 | 0.86 | 0.74 | 0.97 | 88.41 | 79.66 | 8.75 |
| cg14309307 | 6 | PREP | 4.22799E−12 | 1.09 | 0.86 | 0.74 | 0.97 | 88.40 | 80.94 | 7.46 |
| cg18896796 | 10 | COL17A1 | 2.63153E−37 | 1.06 | 0.85 | 0.73 | 0.97 | 96.49 | 91.26 | 5.22 |
| cg23809103 | 7 | ASB15 | 5.08599E−40 | 1.16 | 0.85 | 0.72 | 0.97 | 83.99 | 72.22 | 11.77 |
| cg20899190 | 2 | WIPF1 | 1.77313E−39 | 1.14 | 0.85 | 0.72 | 0.97 | 84.43 | 73.90 | 10.53 |
| cg21092030 | 14 | ASPG | 5.61237E−12 | 1.14 | 0.85 | 0.72 | 0.97 | 78.73 | 68.82 | 9.91 |
| cg01166186 | 14 | MTA1 | 2.68964E−11 | 0.62 | 0.85 | 0.72 | 0.97 | 17.41 | 28.22 | −10.81 |
| cg08134286 | 1 | PER3 | 5.81483E−40 | 1.16 | 0.84 | 0.72 | 0.97 | 85.79 | 74.15 | 11.64 |
| cg10991081 | 1 | RNPEP | 3.20781E−14 | 1.10 | 0.84 | 0.72 | 0.97 | 89.02 | 81.06 | 7.96 |
| cg08766335 | 1 | S100PBP | 7.31277E−12 | 1.10 | 0.84 | 0.72 | 0.97 | 86.66 | 78.77 | 7.90 |
| cg08052226 | 19 | NFIX | 2.42791E−25 | 0.44 | 0.84 | 0.72 | 0.97 | 11.40 | 25.91 | −14.50 |
| cg13378092 | 5 | TRIO | 4.65627E−40 | 1.17 | 0.84 | 0.71 | 0.97 | 80.02 | 68.16 | 11.86 |
| cg15184174 | 11 | TTC17 | 2.32603E−40 | 1.20 | 0.84 | 0.71 | 0.96 | 74.16 | 61.63 | 12.53 |
| cg24400588 | 3 | MAGI1 | 2.81505E−13 | 0.19 | 0.84 | 0.71 | 0.96 | 1.48 | 7.70 | −6.22 |
| cg00765710 | 1 | PGBD2 | 4.97567E−17 | 0.35 | 0.84 | 0.71 | 0.96 | 4.77 | 13.57 | −8.79 |
| cg27269947 | 4 | ELF2 | 7.33627E−42 | 1.24 | 0.83 | 0.71 | 0.96 | 81.13 | 65.41 | 15.72 |
| cg11011922 | 22 | TOMM22 | 5.6971E−40 | 1.16 | 0.83 | 0.71 | 0.96 | 82.84 | 71.18 | 11.66 |
| cg21063991 | 2 | CASP10 | 3.68073E−13 | 1.11 | 0.83 | 0.71 | 0.96 | 86.34 | 77.89 | 8.45 |
| cg18138866 | 1 | DNAJC8 | 1.72984E−11 | 0.27 | 0.83 | 0.71 | 0.96 | 2.15 | 8.11 | −5.96 |
| cg23522895 | 17 | FN3KRP | 1.07918E−40 | 1.19 | 0.83 | 0.70 | 0.96 | 83.31 | 70.06 | 13.26 |
| cg27333460 | 6 | CDYL | 4.2622E−40 | 1.17 | 0.83 | 0.70 | 0.96 | 83.98 | 72.04 | 11.94 |
| cg00950381 | 17 | QRICH2 | 2.45978E−12 | 1.13 | 0.83 | 0.70 | 0.96 | 81.55 | 72.12 | 9.43 |
| cg03966514 | 13 | SPG20 | 5.00424E−23 | 0.34 | 0.83 | 0.70 | 0.96 | 5.50 | 16.39 | −10.89 |
| cg10927394 | 19 | ZNF274 | 8.53576E−30 | 0.28 | 0.83 | 0.70 | 0.96 | 4.74 | 17.01 | −12.27 |
| cg02641420 | 17 | PITPNC1 | 8.79604E−42 | 1.26 | 0.83 | 0.70 | 0.96 | 74.27 | 58.71 | 15.56 |
| cg10949773 | 9 | KCNT1 | 1.79609E−40 | 1.21 | 0.83 | 0.70 | 0.96 | 74.00 | 61.23 | 12.77 |
| cg26451345 | 6 | GPR115 | 4.14791E−14 | 1.13 | 0.83 | 0.70 | 0.96 | 83.91 | 74.43 | 9.48 |
| cg21977555 | 10 | CAMK1D | 2.56275E−14 | 1.10 | 0.83 | 0.70 | 0.96 | 89.04 | 81.06 | 7.97 |
| cg18972013 | 17 | GAA | 2.50441E−37 | 1.06 | 0.83 | 0.70 | 0.96 | 96.47 | 91.19 | 5.28 |
| cg16127683 | 15 | EIF2AK4 | 3.67765E−14 | 0.92 | 0.83 | 0.70 | 0.96 | 85.72 | 93.68 | −7.96 |
| cg07534705 | 7 | NCAPG2 | 5.39026E−41 | 1.20 | 0.82 | 0.69 | 0.96 | 82.40 | 68.49 | 13.91 |
| cg08105295 | 3 | SSUH2 | 5.46059E−41 | 1.23 | 0.82 | 0.69 | 0.96 | 75.02 | 61.12 | 13.89 |
| cg09352912 | 21 | TCP10L | 9.22743E−40 | 1.15 | 0.82 | 0.69 | 0.96 | 83.36 | 72.18 | 11.18 |
| cg22913550 | 5 | NIPAL4 | 1.26977E−39 | 1.14 | 0.82 | 0.69 | 0.96 | 86.14 | 75.27 | 10.87 |
| cg17192132 | 2 | PCBP1-AS1 | 6.6733E−13 | 1.11 | 0.82 | 0.69 | 0.96 | 85.71 | 77.17 | 8.54 |
| cg07235453 | 8 | TAF2 | 1.866E−12 | 0.32 | 0.82 | 0.69 | 0.96 | 3.21 | 9.93 | −6.71 |
| cg15074995 | 4 | CEP135 | 8.79361E−14 | 1.10 | 0.82 | 0.69 | 0.95 | 88.00 | 79.85 | 8.15 |
| cg18823832 | 5 | ZNF354C | 6.38042E−40 | 1.15 | 0.82 | 0.69 | 0.95 | 88.41 | 76.86 | 11.55 |
| cg08805575 | 10 | TLX1NB | 7.29046E−40 | 1.15 | 0.82 | 0.69 | 0.95 | 87.35 | 75.93 | 11.42 |
| cg20821466 | 7 | RNF216 | 1.35941E−39 | 1.15 | 0.82 | 0.69 | 0.95 | 82.25 | 71.45 | 10.80 |
| cg14678442 | 17 | NOG | 5.21706E−30 | 0.40 | 0.82 | 0.69 | 0.95 | 11.56 | 28.96 | −17.40 |
| cg15375051 | 4 | APBB2 | 1.83598E−41 | 1.45 | 0.82 | 0.68 | 0.95 | 47.75 | 32.86 | 14.89 |
| cg07006961 | 1 | RHBDL2 | 2.28536E−11 | 1.18 | 0.82 | 0.68 | 0.95 | 70.82 | 59.83 | 10.99 |
| cg02948855 | 10 | KNDC1 | 4.46128E−38 | 1.08 | 0.82 | 0.68 | 0.95 | 94.05 | 86.87 | 7.18 |
| cg23167148 | 9 | TOR2A | 5.77194E−13 | 0.31 | 0.82 | 0.68 | 0.95 | 3.15 | 10.01 | −6.86 |
| cg14809932 | 6 | UBD | 2.2628E−40 | 1.20 | 0.81 | 0.68 | 0.95 | 75.52 | 62.97 | 12.55 |
| cg27052954 | 1 | JAK1 | 7.84907E−39 | 1.12 | 0.81 | 0.68 | 0.95 | 87.19 | 78.17 | 9.02 |
| cg01869503 | 2 | SLC4A5 | 8.32165E−13 | 1.11 | 0.81 | 0.68 | 0.95 | 86.49 | 78.21 | 8.28 |
| cg02283796 | 19 | AURKC | 4.77954E−13 | 1.10 | 0.81 | 0.68 | 0.95 | 87.65 | 79.63 | 8.02 |
| cg11051192 | 12 | PHC1 | 3.69919E−11 | 1.10 | 0.81 | 0.68 | 0.95 | 86.71 | 79.09 | 7.62 |
| cg22580292 | 10 | PPP2R2D | 9.36701E−38 | 1.07 | 0.81 | 0.68 | 0.95 | 95.76 | 89.39 | 6.37 |
| cg17436861 | 5 | DBN1 | 4.13545E−14 | 0.42 | 0.81 | 0.68 | 0.95 | 6.18 | 14.67 | −8.48 |
| cg04518477 | 16 | METTL9 | 2.32751E−14 | 0.46 | 0.81 | 0.68 | 0.95 | 7.99 | 17.28 | −9.29 |
| cg05156850 | 2 | KRCC1 | 5.39702E−13 | 0.58 | 0.81 | 0.68 | 0.95 | 15.23 | 26.36 | −11.13 |
| cg19614811 | 3 | GPR15 | 1.18927E−15 | 1.21 | 0.81 | 0.67 | 0.95 | 73.23 | 60.72 | 12.51 |
| cg12720889 | 11 | OPCML | 2.56736E−40 | 1.19 | 0.81 | 0.67 | 0.95 | 78.80 | 66.37 | 12.43 |
| cg23921871 | 12 | ACACB | 2.7219E−15 | 1.20 | 0.81 | 0.67 | 0.95 | 73.91 | 61.68 | 12.24 |
| cg04912506 | 18 | SPIRE1 | 4.85576E−40 | 1.17 | 0.81 | 0.67 | 0.95 | 81.81 | 69.99 | 11.81 |
| cg07464621 | 6 | DPPA5 | 1.00442E−39 | 1.16 | 0.81 | 0.67 | 0.95 | 81.06 | 69.96 | 11.10 |
| cg03698668 | 16 | UBE2I | 6.89839E−13 | 1.15 | 0.81 | 0.67 | 0.95 | 79.17 | 68.98 | 10.20 |
| cg10034863 | 18 | ZNF516 | 2.23578E−14 | 1.14 | 0.81 | 0.67 | 0.95 | 82.52 | 72.55 | 9.97 |
| cg05983695 | 3 | CACNA2D2 | 5.0459E−11 | 1.08 | 0.81 | 0.67 | 0.95 | 90.36 | 83.88 | 6.48 |
| cg14711570 | 18 | CCDC68 | 5.37258E−41 | 1.24 | 0.81 | 0.67 | 0.95 | 71.09 | 57.18 | 13.91 |
| cg10802371 | 3 | RNF13 | 2.63932E−13 | 1.26 | 0.81 | 0.67 | 0.95 | 62.75 | 49.94 | 12.81 |
| cg18219522 | 11 | MAML2 | 2.69311E−12 | 1.29 | 0.81 | 0.67 | 0.95 | 55.89 | 43.21 | 12.69 |
| cg06681878 | 4 | RASSF6 | 1.20681E−39 | 1.15 | 0.81 | 0.67 | 0.95 | 85.83 | 74.91 | 10.92 |
| cg15324873 | 15 | SIN3A | 5.91439E−13 | 1.16 | 0.81 | 0.67 | 0.95 | 77.17 | 66.52 | 10.65 |
| cg01643856 | 7 | C7orf10 | 3.01875E−15 | 1.14 | 0.81 | 0.67 | 0.95 | 83.58 | 73.51 | 10.07 |
| cg08448284 | 3 | ATP2C1 | 2.35315E−38 | 1.09 | 0.81 | 0.67 | 0.95 | 91.78 | 83.91 | 7.86 |
| cg18843944 | 7 | PTPRN2 | 2.13068E−12 | 1.07 | 0.81 | 0.67 | 0.95 | 93.11 | 87.16 | 5.95 |
| cg00190549 | 9 | DEC1 | 7.39595E−42 | 1.29 | 0.80 | 0.67 | 0.94 | 69.76 | 54.05 | 15.71 |
| cg19182918 | 17 | KRT40 | 8.52868E−15 | 1.36 | 0.80 | 0.67 | 0.94 | 52.88 | 38.97 | 13.90 |
| cg25900902 | 17 | ACAP1 | 2.33998E−40 | 1.19 | 0.80 | 0.67 | 0.94 | 78.07 | 65.55 | 12.52 |
| cg23547759 | 19 | ILVBL | 2.20829E−11 | 1.18 | 0.80 | 0.67 | 0.94 | 71.14 | 60.19 | 10.95 |
| cg05323272 | 1 | TTC39A | 1.60813E−39 | 1.15 | 0.80 | 0.67 | 0.94 | 82.08 | 71.45 | 10.63 |
| cg11494887 | 11 | CEP164 | 6.49907E−12 | 1.12 | 0.80 | 0.67 | 0.94 | 83.64 | 74.91 | 8.74 |
| cg00985725 | 12 | ACACB | 1.10191E−12 | 1.10 | 0.80 | 0.67 | 0.94 | 87.84 | 80.01 | 7.83 |
| cg15630265 | 11 | SHANK2 | 4.88549E−38 | 1.08 | 0.80 | 0.67 | 0.94 | 92.43 | 85.34 | 7.08 |
| cg16479173 | 11 | MADD | 2.02704E−11 | 0.57 | 0.80 | 0.67 | 0.94 | 13.09 | 22.83 | −9.73 |
| cg23136742 | 2 | KCNK3 | 3.96918E−11 | 0.88 | 0.80 | 0.67 | 0.94 | 73.67 | 83.91 | −10.24 |
| cg08551532 | 19 | DLL3 | 3.89439E−41 | 1.94 | 0.80 | 0.66 | 0.94 | 29.26 | 15.06 | 14.21 |
| cg08410850 | 6 | POLH | 1.14764E−40 | 1.20 | 0.80 | 0.66 | 0.94 | 79.30 | 66.10 | 13.20 |
| cg10218880 | 4 | FBXW7 | 2.32056E−40 | 1.17 | 0.80 | 0.66 | 0.94 | 85.60 | 73.08 | 12.53 |
| cg26937534 | 4 | OCIAD1 | 3.38766E−40 | 1.18 | 0.80 | 0.66 | 0.94 | 78.48 | 66.32 | 12.16 |
| cg26228812 | 6 | WDR27 | 4.14951E−40 | 1.16 | 0.80 | 0.66 | 0.94 | 86.61 | 74.64 | 11.97 |
| cg20667709 | 2 | AGAP1; AGAP1 | 2.42714E−14 | 1.19 | 0.80 | 0.66 | 0.94 | 73.51 | 61.60 | 11.92 |
| cg07256095 | 10 | LOC105376360 | 9.39903E−13 | 1.13 | 0.80 | 0.66 | 0.94 | 81.66 | 72.09 | 9.57 |
| cg11411929 | 1 | CPT2 | 4.01196E−11 | 1.13 | 0.80 | 0.66 | 0.94 | 80.12 | 70.89 | 9.23 |
| cg04496250 | 16 | CDR2 | 1.15567E−11 | 1.11 | 0.80 | 0.66 | 0.94 | 84.76 | 76.41 | 8.35 |
| cg09964933 | 17 | RPTOR; RPTOR | 5.1303E−11 | 1.09 | 0.80 | 0.66 | 0.94 | 88.15 | 80.99 | 7.16 |
| cg10246581 | 16 | CMIP; CMIP | 1.51988E−37 | 1.06 | 0.80 | 0.66 | 0.94 | 96.17 | 90.33 | 5.84 |
| cg05313261 | 16 | MAPK3; MAPK3; MAPK3 | 1.21368E−12 | 0.46 | 0.80 | 0.66 | 0.94 | 6.92 | 15.16 | −8.24 |
| cg17174775 | 7 | AASS | 3.95533E−17 | 0.40 | 0.80 | 0.66 | 0.94 | 6.22 | 15.71 | −9.49 |
| cg24575067 | 20 | CD40; CD40 | 1.23692E−13 | 0.54 | 0.80 | 0.66 | 0.94 | 12.03 | 22.46 | −10.44 |
| cg10394510 | 3 | ANKRD28 | 7.12265E−41 | 1.21 | 0.80 | 0.66 | 0.94 | 80.07 | 66.43 | 13.65 |
| cg04370102 | 9 | MRRF; MRRF; MRRF | 1.11903E−15 | 1.21 | 0.80 | 0.66 | 0.94 | 72.83 | 60.17 | 12.66 |
| cg08466075 | 1 | SDCCAG8 | 7.28629E−15 | 1.20 | 0.80 | 0.66 | 0.94 | 73.51 | 61.36 | 12.15 |
| cg06139029 | 3 | PEX5L | 9.8268E−40 | 1.16 | 0.80 | 0.66 | 0.94 | 82.29 | 71.16 | 11.12 |
| cg22882684 | 3 | TNFSF10 | 1.31686E−39 | 1.15 | 0.80 | 0.66 | 0.94 | 82.47 | 71.64 | 10.83 |
| cg10778298 | 1 | PEX14 | 5.18237E−39 | 1.12 | 0.80 | 0.66 | 0.94 | 86.11 | 76.66 | 9.45 |
| cg15549767 | 17 | BRIP1 | 7.88063E−14 | 1.13 | 0.80 | 0.66 | 0.94 | 84.06 | 74.71 | 9.35 |
| cg00239847 | 20 | PMEPA1; PMEPA1; PMEPA1; | 1.18086E−14 | 1.10 | 0.80 | 0.66 | 0.94 | 88.63 | 80.40 | 8.22 |
| | | PMEPA1; PMEPA1; PMEPA1 | | | | | | | | |
| cg02028941 | 7 | ACTR3B; ACTR3B; ACTR3B; | 4.08519E−12 | 1.10 | 0.80 | 0.66 | 0.94 | 86.72 | 78.75 | 7.97 |
| | | ACTR3B | | | | | | | | |
| cg09971671 | 12 | ZNF664-FAM101A | 5.80986E−14 | 1.09 | 0.80 | 0.66 | 0.94 | 89.96 | 82.42 | 7.54 |
| cg17113376 | 10 | CDNF | 4.41592E−12 | 1.09 | 0.80 | 0.66 | 0.94 | 89.63 | 82.56 | 7.07 |
| cg05706173 | 1 | PRKCZ; PRKCZ; PRKCZ | 5.81802E−38 | 1.08 | 0.80 | 0.66 | 0.94 | 92.55 | 85.66 | 6.89 |
| cg15909801 | 22 | ATXN10; ATXN10 | 5.57846E−12 | 0.42 | 0.80 | 0.66 | 0.94 | 5.48 | 12.93 | −7.45 |
| cg00001793 | 12 | ETV6 | 1.30343E−42 | 1.30 | 0.79 | 0.65 | 0.94 | 74.70 | 57.48 | 17.23 |
| cg14759548 | 6 | PEX7 | 6.21746E−41 | 1.20 | 0.79 | 0.65 | 0.94 | 81.37 | 67.60 | 13.77 |
| cg13931996 | 1 | AJAP1 | 1.61072E−39 | 1.14 | 0.79 | 0.65 | 0.94 | 88.58 | 77.95 | 10.63 |
| cg14165633 | 20 | MAVS | 2.8104E−39 | 1.13 | 0.79 | 0.65 | 0.94 | 86.46 | 76.39 | 10.07 |
| cg10405889 | 6 | SYNGAP1 | 2.90654E−39 | 1.13 | 0.79 | 0.65 | 0.94 | 86.46 | 76.42 | 10.04 |
| cg19722781 | 19 | ZNF347 | 4.73E−11 | 1.14 | 0.79 | 0.65 | 0.94 | 77.42 | 67.68 | 9.75 |
| cg01445151 | 6 | SLC22A23; SLC22A23 | 1.44475E−12 | 1.13 | 0.79 | 0.65 | 0.94 | 82.23 | 72.87 | 9.35 |
| cg23935220 | 20 | RPRD1B | 8.82365E−14 | 1.12 | 0.79 | 0.65 | 0.94 | 84.20 | 74.91 | 9.29 |
| cg03468524 | 1 | DISC1; DISC1; DISC1; DISC1; | 2.09081E−38 | 1.10 | 0.79 | 0.65 | 0.94 | 90.51 | 82.52 | 7.99 |
| | | DISC1; DISC1; DISC1; DISC1; | | | | | | | | |
| | | DISC1; DISC1; DISC1; DISC1; | | | | | | | | |
| | | DISC1; DISC1; DISC1; | | | | | | | | |
| | | TSNAX-DISC1; TSNAX-DISC1; | | | | | | | | |
| | | TSNAX-DISC1; TSNAX-DISC1; | | | | | | | | |
| | | TSNAX-DISC1; TSNAX-DISC1 | | | | | | | | |
| cg11302655 | 10 | MGMT | 3.19762E−11 | 1.07 | 0.79 | 0.65 | 0.94 | 91.09 | 84.77 | 6.31 |
| cg06301252 | 7 | PTPRN2 | 5.86994E−53 | 1.70 | 0.79 | 0.65 | 0.94 | 81.34 | 47.72 | 33.62 |
| cg24984195 | 16 | UBE2I | 3.36141E−43 | 1.65 | 0.79 | 0.65 | 0.94 | 46.46 | 28.09 | 18.38 |
| cg14553042 | 5 | CPEB4 | 7.29516E−41 | 1.22 | 0.79 | 0.65 | 0.94 | 74.40 | 60.77 | 13.62 |
| cg04762820 | 14 | RGS6; RGS6; RGS6; RGS6; RGS6; | 1.8145E−15 | 1.17 | 0.79 | 0.65 | 0.94 | 78.69 | 67.27 | 11.42 |
| | | RGS6; RGS6; RGS6; RGS6; RGS6 | | | | | | | | |
| cg10235170 | 17 | ADORA2B | 5.3693E−12 | 1.19 | 0.79 | 0.65 | 0.94 | 71.18 | 59.93 | 11.24 |
| cg09049417 | 8 | FZD3 | 1.38895E−39 | 1.15 | 0.79 | 0.65 | 0.94 | 83.17 | 72.39 | 10.78 |
| cg13581510 | 6 | AHI1; AHI1; AHI1; AHI1; | 3.3203E−14 | 1.15 | 0.79 | 0.65 | 0.94 | 79.67 | 69.05 | 10.62 |
| | | LINC00271 | | | | | | | | |
| cg15742818 | 17 | KSR1 | 5.51469E−39 | 1.12 | 0.79 | 0.65 | 0.94 | 87.82 | 78.44 | 9.38 |
| cg16053745 | 3 | C3orf43 | 7.07099E−39 | 1.12 | 0.79 | 0.65 | 0.94 | 86.72 | 77.60 | 9.13 |
| cg01117844 | 14 | NEMF; NEMF | 1.6378E−14 | 1.11 | 0.79 | 0.65 | 0.94 | 87.74 | 79.29 | 8.45 |
| cg19614456 | 1 | GABPB2 | 1.10807E−14 | 1.11 | 0.79 | 0.65 | 0.94 | 88.09 | 79.69 | 8.40 |
| cg19942268 | 9 | RGS3 | 5.36623E−12 | 1.11 | 0.79 | 0.65 | 0.94 | 85.61 | 77.37 | 8.24 |
| cg05088513 | 18 | AFG3L2 | 1.64467E−12 | 1.10 | 0.79 | 0.65 | 0.94 | 87.77 | 79.98 | 7.80 |
| cg06302399 | 8 | C8orf37 | 3.17976E−11 | 1.10 | 0.79 | 0.65 | 0.94 | 86.74 | 79.10 | 7.64 |
| cg02903958 | 16 | CFAP20 | 6.19624E−11 | 1.08 | 0.79 | 0.65 | 0.94 | 89.78 | 83.15 | 6.63 |
| cg23711881 | 14 | C14orf102; C14orf102 | 4.45942E−40 | 1.16 | 0.79 | 0.64 | 0.93 | 84.34 | 72.44 | 11.90 |
| cg18385205 | 1 | PRAMEF22 | 1.7901E−39 | 1.14 | 0.79 | 0.64 | 0.93 | 85.10 | 74.57 | 10.53 |
| cg14149503 | 14 | RNASE12 | 8.36606E−15 | 1.12 | 0.79 | 0.64 | 0.93 | 85.68 | 76.48 | 9.20 |
| cg21670679 | 6 | ZFAND3 | 2.6517E−12 | 1.12 | 0.79 | 0.64 | 0.93 | 83.69 | 74.81 | 8.88 |
| cg02779164 | 18 | SETBP1 | 5.25672E−13 | 1.10 | 0.79 | 0.64 | 0.93 | 88.12 | 80.27 | 7.85 |
| cg18180456 | 4 | JADE1; JADE1; JADE1; JADE1; | 3.20879E−13 | 0.34 | 0.79 | 0.64 | 0.93 | 3.61 | 10.74 | −7.13 |
| | | JADE1; JADE1; JADE1; JADE1 | | | | | | | | |
| cg13305245 | 8 | NRG1 | 1.27064E−41 | 1.33 | 0.79 | 0.64 | 0.93 | 60.83 | 45.61 | 15.23 |
| cg17314277 | 1 | NMNAT2 | 6.13134E−41 | 1.21 | 0.79 | 0.64 | 0.93 | 80.76 | 66.98 | 13.79 |
| cg10618720 | 8 | PTK2; PTK2; PTK2 | 3.32343E−39 | 1.13 | 0.79 | 0.64 | 0.93 | 87.97 | 78.07 | 9.90 |
| cg01609612 | 6 | ATXN1; ATXN1 | 6.32859E−11 | 1.10 | 0.79 | 0.64 | 0.93 | 86.03 | 78.31 | 7.72 |
| cg06634750 | 16 | PAPD5; PAPD5 | 2.25208E−12 | 0.38 | 0.79 | 0.64 | 0.93 | 4.47 | 11.66 | −7.19 |
| cg19125926 | 11 | LOC101929089; LOC101929089; | 2.23522E−23 | 0.46 | 0.79 | 0.64 | 0.93 | 12.17 | 26.35 | −14.17 |
| | | LOC101929089; LOC101929089; | | | | | | | | |
| | | TMEM25; TMEM25; TMEM25; | | | | | | | | |
| | | TMEM25; TMEM25; TMEM25; | | | | | | | | |
| | | LOC101929089 | | | | | | | | |
| cg25269392 | 1 | KIRREL; KIRREL | 5.38268E−13 | 0.25 | 0.79 | 0.64 | 0.93 | 2.15 | 8.58 | −6.43 |
| cg17359931 | 3 | KAT2B | 2.43827E−15 | 1.51 | 0.79 | 0.64 | 0.93 | 40.55 | 26.91 | 13.64 |
| cg12518318 | 7 | KCND2 | 1.7067E−40 | 1.18 | 0.79 | 0.64 | 0.93 | 83.86 | 71.04 | 12.82 |
| cg13292607 | 10 | CTBP2; CTBP2 | 8.12365E−15 | 1.19 | 0.79 | 0.64 | 0.93 | 74.95 | 63.09 | 11.86 |
| cg06564376 | 7 | ATXN7L1; ATXN7L1 | 5.70865E−40 | 1.17 | 0.79 | 0.64 | 0.93 | 80.25 | 68.60 | 11.66 |
| cg13983551 | 21 | KRTAP12-2; TSPEAR; TSPEAR | 1.55267E−11 | 1.21 | 0.79 | 0.64 | 0.93 | 67.02 | 55.48 | 11.54 |
| cg08957853 | 3 | RBM5-AS1; RBM5; RBM5; RBM5 | 9.99018E−40 | 1.15 | 0.79 | 0.64 | 0.93 | 83.78 | 72.67 | 11.11 |
| cg12700695 | 22 | ASPHD2 | 2.05987E−15 | 1.16 | 0.79 | 0.64 | 0.93 | 80.01 | 68.92 | 11.09 |
| cg08136809 | 19 | TMEM91; TMEM91; TMEM91; | 5.38639E−12 | 1.71 | 0.79 | 0.64 | 0.93 | 24.64 | 14.44 | 10.20 |
| | | TMEM91; TMEM91; TMEM91; | | | | | | | | |
| | | TMEM91; TMEM91; TMEM91; | | | | | | | | |
| | | TMEM91 | | | | | | | | |
| cg05017821 | 20 | GZF1 | 4.15658E−11 | 1.13 | 0.79 | 0.64 | 0.93 | 79.51 | 70.15 | 9.36 |
| cg21868480 | 11 | CPT1A; CPT1A | 5.68402E−39 | 1.12 | 0.79 | 0.64 | 0.93 | 86.06 | 76.71 | 9.35 |
| cg14884747 | 4 | ZNF330 | 7.76622E−39 | 1.11 | 0.79 | 0.64 | 0.93 | 88.05 | 79.02 | 9.03 |
| cg09608523 | 3 | ROBO1 | 4.76184E−12 | 1.12 | 0.79 | 0.64 | 0.93 | 83.03 | 74.08 | 8.95 |
| cg08126654 | 12 | LOC101927292 | 1.29344E−38 | 1.10 | 0.79 | 0.64 | 0.93 | 90.14 | 81.65 | 8.50 |
| cg25922445 | 8 | WHSC1L1; WHSC1L1 | 1.57669E−13 | 1.10 | 0.79 | 0.64 | 0.93 | 87.48 | 79.25 | 8.23 |
| cg15617336 | 11 | UNC93B1 | 2.08701E−38 | 1.10 | 0.79 | 0.64 | 0.93 | 91.14 | 83.15 | 7.99 |
| cg19709987 | 9 | QSOX2 | 3.35689E−14 | 1.10 | 0.79 | 0.64 | 0.93 | 89.39 | 81.56 | 7.82 |
| cg24383480 | 3 | SCAP | 1.33122E−12 | 1.10 | 0.79 | 0.64 | 0.93 | 88.21 | 80.52 | 7.69 |
| cg15447512 | 1 | AMPD2; AMPD2 | 2.06138E−11 | 0.62 | 0.79 | 0.64 | 0.93 | 18.12 | 29.16 | −11.05 |
| cg04826071 | 15 | TTBK2 | 1.14896E−13 | 0.62 | 0.79 | 0.64 | 0.93 | 21.50 | 34.52 | −13.02 |
| cg03716245 | 6 | MDN1 | 3.96473E−41 | 1.26 | 0.78 | 0.64 | 0.93 | 68.54 | 54.35 | 14.19 |
| cg17764322 | 2 | AGAP1; AGAP1; AGAP1 | 2.6139E−14 | 1.27 | 0.78 | 0.64 | 0.93 | 62.36 | 49.06 | 13.29 |
| cg18148659 | 15 | DENND4A; DENND4A | 3.8379E−13 | 1.21 | 0.78 | 0.64 | 0.93 | 69.67 | 57.69 | 11.99 |
| cg27338087 | 12 | ATXN2; ATXN2; ATXN2; ATXN2 | 1.37915E−39 | 1.15 | 0.78 | 0.64 | 0.93 | 84.17 | 73.38 | 10.79 |
| cg25742300 | 2 | LRP1B | 5.18021E−11 | 1.15 | 0.78 | 0.64 | 0.93 | 75.97 | 65.97 | 10.00 |
| cg07332338 | 8 | TRAPPC9; TRAPPC9 | 4.00126E−39 | 1.13 | 0.78 | 0.64 | 0.93 | 86.20 | 76.49 | 9.71 |
| cg25064458 | 1 | PGM1 | 3.16086E−38 | 1.09 | 0.78 | 0.64 | 0.93 | 91.08 | 83.53 | 7.55 |
| cg20151476 | 7 | PSMG3; PSMG3 | 5.65868E−38 | 1.08 | 0.78 | 0.64 | 0.93 | 93.76 | 86.84 | 6.92 |
| cg14353079 | 9 | SMC5 | 1.99535E−12 | 0.38 | 0.78 | 0.64 | 0.93 | 4.40 | 11.58 | −7.19 |
| cg16930275 | 19 | TRIP10; TRIP10; TRIP10; TRIP10 | 5.31406E−17 | 0.26 | 0.78 | 0.63 | 0.93 | 2.74 | 10.54 | −7.80 |
| cg07481360 | 3 | DLG1; DLG1 | 8.65723E−15 | 1.34 | 0.78 | 0.63 | 0.93 | 55.20 | 41.33 | 13.86 |
| cg18191605 | 20 | SPATA2 | 1.05885E−40 | 1.19 | 0.78 | 0.63 | 0.93 | 81.49 | 68.22 | 13.27 |
| cg27470056 | 1 | IVNS1ABP | 3.4857E−14 | 1.26 | 0.78 | 0.63 | 0.93 | 63.22 | 50.05 | 13.17 |
| cg13226072 | 3 | MECOM; MECOM; MECOM; | 6.43822E−15 | 1.24 | 0.78 | 0.63 | 0.93 | 68.01 | 55.03 | 12.98 |
| | | MECOM; MECOM; MECOM; | | | | | | | | |
| | | MECOM | | | | | | | | |
| cg18077049 | 4 | GLRA3; GLRA3 | 1.49459E−40 | 1.20 | 0.78 | 0.63 | 0.93 | 79.25 | 66.30 | 12.95 |
| cg09240767 | 8 | MYBL1; MYBL1; MYBL1 | 1.44131E−13 | 1.25 | 0.78 | 0.63 | 0.93 | 64.24 | 51.44 | 12.80 |
| cg22291942 | 18 | DTNA; DTNA; DTNA; DTNA; | 2.03261E−40 | 1.18 | 0.78 | 0.63 | 0.93 | 81.96 | 69.31 | 12.66 |
| | | DTNA; DTNA; DTNA; DTNA; | | | | | | | | |
| | | DTNA | | | | | | | | |
| cg09822302 | 1 | C1orf183 | 1.20367E−15 | 1.21 | 0.78 | 0.63 | 0.93 | 73.22 | 60.75 | 12.48 |
| cg14779509 | 7 | JAZF1 | 3.78813E−14 | 1.21 | 0.78 | 0.63 | 0.93 | 70.22 | 57.86 | 12.36 |
| cg12609948 | 1 | CAMTA1 | 2.79017E−40 | 1.18 | 0.78 | 0.63 | 0.93 | 79.47 | 67.11 | 12.35 |
| cg26529323 | 12 | R3HDM2 | 5.9957E−40 | 1.17 | 0.78 | 0.63 | 0.93 | 81.80 | 70.19 | 11.61 |
| cg08363235 | 10 | SGMS1 | 4.99432E−11 | 1.20 | 0.78 | 0.63 | 0.93 | 68.26 | 57.12 | 11.14 |
| cg18658860 | 12 | PRH1-PRR4; PRH1; PRH1 | 1.97086E−11 | 1.18 | 0.78 | 0.63 | 0.93 | 72.22 | 61.40 | 10.82 |
| cg10626169 | 7 | ABCA13 | 1.34307E−12 | 1.17 | 0.78 | 0.63 | 0.93 | 75.84 | 65.09 | 10.75 |
| cg19491388 | 14 | TTLL5 | 4.91441E−15 | 1.15 | 0.78 | 0.63 | 0.93 | 80.69 | 70.02 | 10.66 |
| cg01868896 | 15 | MYO5C | 2.29757E−12 | 1.14 | 0.78 | 0.63 | 0.93 | 80.01 | 70.22 | 9.79 |
| cg09072447 | 9 | WNK2; WNK2 | 2.33103E−12 | 1.13 | 0.78 | 0.63 | 0.93 | 81.14 | 71.60 | 9.53 |
| cg23632539 | 1 | MIR5096; GNG4; GNG4; GNG4 | 2.28312E−13 | 1.12 | 0.78 | 0.63 | 0.93 | 84.24 | 75.11 | 9.13 |
| cg12188454 | 15 | PRC1-AS1; PRC1; PRC1; PRC1 | 2.82496E−12 | 1.11 | 0.78 | 0.63 | 0.93 | 85.74 | 77.43 | 8.31 |
| cg21732004 | 11 | ANO9 | 2.27031E−13 | 1.10 | 0.78 | 0.63 | 0.93 | 87.50 | 79.33 | 8.17 |
| cg00666842 | 2 | SMYD1 | 7.5646E−12 | 1.08 | 0.78 | 0.63 | 0.93 | 89.65 | 82.67 | 6.98 |
| cg01566592 | 8 | RIMS2 | 3.25438E−12 | 0.46 | 0.78 | 0.63 | 0.93 | 7.03 | 15.14 | −8.11 |
| cg19697107 | 9 | RALGPS1; RALGPS1; RALGPS1 | 6.25712E−15 | 0.46 | 0.78 | 0.63 | 0.93 | 8.35 | 17.98 | −9.63 |
| cg15071391 | 13 | MTMR6 | 7.86448E−41 | 1.22 | 0.78 | 0.63 | 0.92 | 75.69 | 62.13 | 13.55 |
| cg24037795 | 7 | MIR335; MEST | 8.74227E−41 | 1.20 | 0.78 | 0.63 | 0.92 | 81.54 | 68.09 | 13.45 |
| cg07104666 | 1 | PSMB2 | 1.6392E−40 | 1.20 | 0.78 | 0.63 | 0.92 | 78.28 | 65.42 | 12.86 |
| cg00587465 | 19 | APOC1P1; APOC1P1; APOC1P1 | 3.66592E−12 | 1.29 | 0.78 | 0.63 | 0.92 | 56.22 | 43.61 | 12.61 |
| cg08316054 | 8 | TRAPPC9; TRAPPC9 | 7.21239E−12 | 1.26 | 0.78 | 0.63 | 0.92 | 60.36 | 48.09 | 12.27 |
| cg20655737 | 10 | C10orf67 | 1.73107E−11 | 1.47 | 0.78 | 0.63 | 0.92 | 36.49 | 24.90 | 11.59 |
| cg00920668 | 1 | LOC148696 | 6.83559E−15 | 1.16 | 0.78 | 0.63 | 0.92 | 79.60 | 68.70 | 10.91 |
| cg27638428 | 5 | LOC285696 | 2.28613E−15 | 1.15 | 0.78 | 0.63 | 0.92 | 80.99 | 70.18 | 10.81 |
| cg02625315 | 3 | RBM15B; RBM15B | 6.39898E−11 | 1.18 | 0.78 | 0.63 | 0.92 | 70.53 | 59.73 | 10.80 |
| cg22397365 | 1 | ST3GAL3; ST3GAL3; ST3GAL3; | 4.86028E−12 | 1.17 | 0.78 | 0.63 | 0.92 | 74.56 | 63.83 | 10.73 |
| | | ST3GAL3; ST3GAL3; ST3GAL3; | | | | | | | | |
| | | ST3GAL3; ST3GAL3; ST3GAL3; | | | | | | | | |
| | | ST3GAL3 | | | | | | | | |
| cg05537387 | 7 | EGFR; EGFR; EGFR; EGFR | 8.56018E−13 | 1.14 | 0.78 | 0.63 | 0.92 | 80.09 | 70.14 | 9.95 |
| cg08821528 | 8 | DLGAP2; DLGAP2 | 2.50888E−11 | 1.14 | 0.78 | 0.63 | 0.92 | 78.74 | 69.12 | 9.62 |
| cg14840429 | 10 | HABP2 | 5.61726E−11 | 1.12 | 0.78 | 0.63 | 0.92 | 82.08 | 73.34 | 8.73 |
| cg11466301 | 16 | WWP2; WWP2; WWP2; WWP2 | 5.88486E−15 | 1.10 | 0.78 | 0.63 | 0.92 | 88.92 | 80.68 | 8.24 |
| cg01925521 | 4 | CPE | 1.6809E−11 | 1.09 | 0.78 | 0.63 | 0.92 | 88.09 | 80.74 | 7.35 |
| cg27555525 | 19 | PTPRS; PTPRS; PTPRS; PTPRS | 5.70452E−14 | 1.07 | 0.78 | 0.63 | 0.92 | 93.48 | 87.26 | 6.22 |
| cg23306063 | 19 | TYK2 | 3.09069E−11 | 0.92 | 0.78 | 0.63 | 0.92 | 84.31 | 91.85 | −7.54 |
| cg24707399 | 19 | RABAC1 | 4.98047E−13 | 0.40 | 0.78 | 0.63 | 0.92 | 5.24 | 12.98 | −7.74 |
| cg25306499 | 19 | PTPRS; PTPRS; PTPRS; PTPRS | 4.6772E−11 | 0.56 | 0.78 | 0.63 | 0.92 | 11.63 | 20.76 | −9.12 |
| cg17779336 | 2 | SF3B14 | 6.92407E−14 | 0.42 | 0.77 | 0.62 | 0.92 | 5.97 | 14.29 | −8.32 |
| cg17237506 | 7 | ZPBP; ZPBP | 1.17918E−14 | 0.47 | 0.77 | 0.62 | 0.92 | 8.41 | 17.97 | −9.56 |
| cg03156546 | 16 | TNRC6A | 7.2807E−13 | 1.41 | 0.77 | 0.62 | 0.92 | 44.22 | 31.39 | 12.82 |
| cg05632277 | 3 | STAG1 | 4.83234E−14 | 1.14 | 0.77 | 0.62 | 0.92 | 82.15 | 72.21 | 9.94 |
| cg22426789 | 1 | CENPF | 6.61778E−12 | 1.10 | 0.77 | 0.62 | 0.92 | 87.27 | 79.54 | 7.73 |
| cg19411922 | 8 | PAG1 | 5.99309E−13 | 1.19 | 0.77 | 0.62 | 0.92 | 72.48 | 60.99 | 11.49 |
| cg24332696 | 3 | ATP13A3 | 1.35173E−39 | 1.15 | 0.77 | 0.62 | 0.92 | 81.85 | 71.04 | 10.81 |
| cg02796420 | 2 | EIF5B | 6.47846E−12 | 1.16 | 0.77 | 0.62 | 0.92 | 75.42 | 64.90 | 10.52 |
| cg09106154 | 16 | CMTM1; CMTM1; CMTM1; | 1.81302E−39 | 1.13 | 0.77 | 0.62 | 0.92 | 90.87 | 80.36 | 10.51 |
| | | CMTM1; CMTM1; CMTM1; | | | | | | | | |
| | | CMTM1; CMTM1 | | | | | | | | |
| cg18699226 | 7 | PDE1C | 1.84872E−11 | 1.16 | 0.77 | 0.62 | 0.92 | 75.59 | 65.31 | 10.28 |
| cg20558008 | 4 | ABCG2 | 1.132E−11 | 1.12 | 0.77 | 0.62 | 0.92 | 82.95 | 74.13 | 8.82 |
| cg00587635 | 6 | RPS6KA2; RPS6KA2 | 5.86239E−13 | 1.11 | 0.77 | 0.62 | 0.92 | 86.01 | 77.53 | 8.48 |
| cg04762397 | 13 | MRPS31 | 2.26841E−11 | 0.39 | 0.77 | 0.62 | 0.92 | 4.26 | 11.01 | −6.75 |
| cg12084602 | 15 | WASH3P | 1.23103E−15 | 0.34 | 0.77 | 0.62 | 0.92 | 4.29 | 12.44 | −8.16 |
| cg09033131 | 11 | MPZL2; MPZL2 | 8.56604E−17 | 0.35 | 0.77 | 0.62 | 0.92 | 4.78 | 13.51 | −8.73 |
| cg24139898 | 19 | HPN; HPN | 3.91709E−23 | 0.39 | 0.77 | 0.62 | 0.92 | 7.71 | 19.77 | −12.06 |
| cg19705300 | 1 | PRDM2 | 1.39086E−41 | 1.30 | 0.77 | 0.62 | 0.92 | 65.81 | 50.66 | 15.15 |
| cg13896818 | 3 | TRIM71 | 2.6911E−15 | 1.20 | 0.77 | 0.62 | 0.92 | 73.84 | 61.57 | 12.27 |
| cg05533783 | 18 | PIAS2; PIAS2 | 4.28821E−40 | 1.17 | 0.77 | 0.62 | 0.92 | 80.99 | 69.05 | 11.94 |
| cg25216910 | 6 | RNGTT | 1.92974E−14 | 1.16 | 0.77 | 0.62 | 0.92 | 79.39 | 68.62 | 10.77 |
| cg19049013 | 4 | SNX25 | 1.48533E−39 | 1.14 | 0.77 | 0.62 | 0.92 | 84.96 | 74.25 | 10.71 |
| cg10148694 | 22 | CBY1; CBY1 | 4.37554E−14 | 1.15 | 0.77 | 0.62 | 0.92 | 79.53 | 68.94 | 10.60 |
| cg15598217 | 17 | ZZEF1 | 4.02164E−11 | 1.16 | 0.77 | 0.62 | 0.92 | 73.76 | 63.33 | 10.43 |
| cg19958024 | 6 | LSM2 | 9.32297E−14 | 1.15 | 0.77 | 0.62 | 0.92 | 79.78 | 69.37 | 10.41 |
| cg27257926 | 6 | TRIM27 | 2.40262E−14 | 1.14 | 0.77 | 0.62 | 0.92 | 81.76 | 71.62 | 10.15 |
| cg14536433 | 19 | SLC6A16 | 6.25025E−15 | 1.14 | 0.77 | 0.62 | 0.92 | 83.21 | 73.20 | 10.01 |
| cg07665222 | 11 | ACRV1; ACRV1; ACRV1; ACRV1; | 9.57029E−15 | 1.14 | 0.77 | 0.62 | 0.92 | 83.03 | 73.05 | 9.98 |
| | | ACRV1; ACRV1; ACRV1; ACRV1; | | | | | | | | |
| | | ACRV1 | | | | | | | | |
| cg09458656 | 16 | NDE1; NDE1 | 3.3152E−39 | 1.13 | 0.77 | 0.62 | 0.92 | 84.64 | 74.74 | 9.90 |
| cg10435675 | 10 | FANK1-AS1; FANK1 | 3.09736E−12 | 1.13 | 0.77 | 0.62 | 0.92 | 82.25 | 73.03 | 9.22 |
| cg19053142 | 11 | C11orf52 | 3.37936E−11 | 1.12 | 0.77 | 0.62 | 0.92 | 81.50 | 72.54 | 8.96 |
| cg19377674 | 15 | DIS3L; DIS3L | 2.86409E−14 | 1.11 | 0.77 | 0.62 | 0.92 | 86.38 | 77.56 | 8.82 |
| cg25826721 | 10 | FANK1 | 2.69291E−11 | 1.08 | 0.77 | 0.62 | 0.92 | 89.27 | 82.36 | 6.92 |
| cg13549962 | 1 | KDM5B | 2.69265E−42 | 1.30 | 0.77 | 0.61 | 0.92 | 71.66 | 55.06 | 16.60 |
| cg01450606 | 8 | PPP2R2A | 4.76604E−41 | 1.26 | 0.77 | 0.61 | 0.92 | 68.70 | 54.68 | 14.02 |
| cg18100502 | 6 | HACE1 | 7.92655E−41 | 1.22 | 0.77 | 0.61 | 0.92 | 74.72 | 61.17 | 13.55 |
| cg25192916 | 18 | MBD2; MBD2 | 1.40345E−40 | 1.19 | 0.77 | 0.61 | 0.92 | 80.81 | 67.80 | 13.01 |
| cg04233620 | 21 | ADARB1; ADARB1; ADARB1; | 1.58417E−40 | 1.22 | 0.77 | 0.61 | 0.92 | 71.90 | 59.01 | 12.89 |
| | | ADARB1; ADARB1; ADARB1; | | | | | | | | |
| | | ADARB1 | | | | | | | | |
| cg17617683 | 1 | TMCC2; TMCC2; TMCC2 | 5.6457E−40 | 1.17 | 0.77 | 0.61 | 0.92 | 80.28 | 68.61 | 11.67 |
| cg23243991 | 12 | STYK1 | 2.50748E−11 | 1.53 | 0.77 | 0.61 | 0.92 | 31.69 | 20.71 | 10.98 |
| cg07254055 | 6 | ANKS1A | 1.75266E−11 | 1.18 | 0.77 | 0.61 | 0.92 | 72.49 | 61.69 | 10.80 |
| cg11257935 | 5 | CNOT8 | 4.48959E−14 | 1.16 | 0.77 | 0.61 | 0.92 | 79.09 | 68.40 | 10.70 |
| cg03944375 | 1 | LDLRAD2 | 4.6143E−12 | 1.15 | 0.77 | 0.61 | 0.92 | 77.96 | 67.85 | 10.10 |
| cg00551589 | 16 | PDPK1; PDPK1 | 3.04953E−12 | 1.15 | 0.77 | 0.61 | 0.92 | 78.52 | 68.45 | 10.07 |
| cg12823329 | 14 | MIR494 | 5.28523E−12 | 1.13 | 0.77 | 0.61 | 0.92 | 80.46 | 70.92 | 9.54 |
| cg24892992 | 8 | EXT1 | 2.21341E−14 | 1.12 | 0.77 | 0.61 | 0.92 | 85.79 | 76.74 | 9.05 |
| cg25830945 | 10 | LRRC27; LRRC27; LRRC27; | 4.57687E−15 | 1.11 | 0.77 | 0.61 | 0.92 | 86.80 | 77.86 | 8.94 |
| | | LRRC27 | | | | | | | | |
| cg11403338 | 19 | C19orf71; C19orf28; C19orf28 | 2.13327E−38 | 1.10 | 0.77 | 0.61 | 0.92 | 90.59 | 82.62 | 7.97 |
| cg16563938 | 1 | WDR8 | 8.88897E−38 | 1.07 | 0.77 | 0.61 | 0.92 | 94.09 | 87.66 | 6.43 |
| cg15191010 | 12 | C12orf40 | 2.20051E−13 | 1.07 | 0.77 | 0.61 | 0.92 | 93.69 | 87.70 | 5.99 |
| cg27440050 | 17 | MAPK7; MAPK7; MAPK7; MAPK7 | 2.09743E−12 | 0.53 | 0.77 | 0.61 | 0.92 | 10.41 | 19.79 | −9.37 |
| cg15690347 | 19 | SPIB | 8.61939E−41 | 1.79 | 0.76 | 0.61 | 0.92 | 30.48 | 17.01 | 13.47 |
| cg02699933 | 11 | ARHGAP32 | 4.30054E−40 | 1.16 | 0.76 | 0.61 | 0.92 | 85.23 | 73.30 | 11.93 |
| cg26397352 | 17 | MIR744; MAP2K4; MAP2K4 | 4.40952E−40 | 1.17 | 0.76 | 0.61 | 0.92 | 81.20 | 69.29 | 11.91 |
| cg02167203 | 6 | NFYA; NFYA; LOC221442 | 5.88011E−40 | 1.16 | 0.76 | 0.61 | 0.92 | 84.23 | 72.60 | 11.63 |
| cg23709617 | 10 | KNDC1 | 6.43875E−40 | 1.17 | 0.76 | 0.61 | 0.92 | 79.74 | 68.20 | 11.54 |
| cg14942793 | 19 | DPY19L3 | 2.00775E−13 | 1.16 | 0.76 | 0.61 | 0.92 | 77.47 | 66.69 | 10.78 |
| cg12462499 | 3 | FNDC3B; FNDC3B | 1.81706E−12 | 1.16 | 0.76 | 0.61 | 0.92 | 76.37 | 65.77 | 10.59 |
| cg21025171 | 7 | CHCHD3 | 6.44171E−12 | 1.15 | 0.76 | 0.61 | 0.92 | 77.95 | 67.90 | 10.04 |
| cg14458731 | 9 | COQ4; TRUB2 | 1.50065E−12 | 1.12 | 0.76 | 0.61 | 0.92 | 82.82 | 73.63 | 9.20 |
| cg08382124 | 8 | ZNF623; ZNF623 | 9.61948E−39 | 1.11 | 0.76 | 0.61 | 0.92 | 89.48 | 80.67 | 8.81 |
| cg17148430 | 15 | MYO1E | 1.02388E−38 | 1.11 | 0.76 | 0.61 | 0.92 | 87.80 | 79.06 | 8.74 |
| cg12690016 | 1 | SSU72 | 1.10475E−38 | 1.11 | 0.76 | 0.61 | 0.92 | 88.17 | 79.51 | 8.66 |
| cg08323345 | 10 | UROS | 4.61269E−11 | 1.09 | 0.76 | 0.61 | 0.92 | 88.02 | 80.81 | 7.21 |
| cg19474104 | 17 | UBE2Z | 1.48258E−27 | 0.27 | 0.76 | 0.61 | 0.92 | 4.13 | 15.45 | −11.32 |
| cg06862673 | 17 | CDK12; CDK12 | 6.36865E−18 | 0.53 | 0.76 | 0.61 | 0.92 | 14.81 | 27.93 | −13.12 |
| cg16609878 | 10 | HELLS | 8.23788E−42 | 1.25 | 0.76 | 0.61 | 0.91 | 77.46 | 61.85 | 15.61 |
| cg01469864 | 5 | CPLX2 | 1.26614E−40 | 1.67 | 0.76 | 0.61 | 0.91 | 32.56 | 19.46 | 13.11 |
| cg20174535 | 13 | DLEU1; DLEU1; | 1.37798E−40 | 2.28 | 0.76 | 0.61 | 0.91 | 23.19 | 10.17 | 13.02 |
| | | DLEU2; DLEU1 | | | | | | | | |
| cg14953092 | 7 | LUC7L2; LUC7L2; | 4.9753E−13 | 1.26 | 0.76 | 0.61 | 0.91 | 61.57 | 48.81 | 12.76 |
| | | C7orf55-LUC7L2; LUC7L2 | | | | | | | | |
| cg14606289 | 1 | S100PBP; S100PBP | 1.23103E−15 | 1.20 | 0.76 | 0.61 | 0.91 | 74.20 | 61.78 | 12.42 |
| cg17471939 | 1 | TP73 | 1.42392E−11 | 1.35 | 0.76 | 0.61 | 0.91 | 48.03 | 35.70 | 12.33 |
| cg18929316 | 17 | CBX1; CBX1 | 2.0102E−11 | 1.21 | 0.76 | 0.61 | 0.91 | 65.78 | 54.17 | 11.61 |
| cg03395247 | 20 | HNF4A; HNF4A; HNF4A; HNF4A; | 2.44581E−13 | 1.16 | 0.76 | 0.61 | 0.91 | 77.07 | 66.24 | 10.83 |
| | | HNF4A; HNF4A | | | | | | | | |
| cg06887949 | 4 | LARP7; LARP7 | 3.512E−13 | 1.14 | 0.76 | 0.61 | 0.91 | 80.51 | 70.50 | 10.01 |
| cg13939297 | 12 | HCFC2 | 7.86271E−14 | 1.14 | 0.76 | 0.61 | 0.91 | 82.01 | 72.12 | 9.90 |
| cg04327593 | 19 | USF2; USF2 | 9.33457E−12 | 1.14 | 0.76 | 0.61 | 0.91 | 79.66 | 70.05 | 9.61 |
| cg03244796 | 16 | HSDL1; HSDL1 | 4.47876E−39 | 1.13 | 0.76 | 0.61 | 0.91 | 86.27 | 76.68 | 9.60 |
| cg14928149 | 7 | RBM33 | 1.60452E−11 | 1.12 | 0.76 | 0.61 | 0.91 | 81.97 | 72.98 | 8.99 |
| cg05042959 | 22 | LINC01422 | 1.60155E−12 | 1.12 | 0.76 | 0.61 | 0.91 | 83.79 | 74.85 | 8.94 |
| cg14287198 | 1 | CAMTA1 | 6.1917E−13 | 1.12 | 0.76 | 0.61 | 0.91 | 84.81 | 76.00 | 8.81 |
| cg23324442 | 14 | C14orf72 | 1.52426E−12 | 1.12 | 0.76 | 0.61 | 0.91 | 84.37 | 75.57 | 8.79 |
| cg03599855 | 8 | EXT1 | 3.87721E−13 | 1.11 | 0.76 | 0.61 | 0.91 | 86.19 | 77.70 | 8.49 |
| cg10453541 | 8 | NSMCE2 | 3.07699E−11 | 1.11 | 0.76 | 0.61 | 0.91 | 83.55 | 75.07 | 8.49 |
| cg15123087 | 7 | ARHGEF5 | 1.18569E−12 | 1.10 | 0.76 | 0.61 | 0.91 | 88.20 | 80.49 | 7.71 |
| cg25383309 | 10 | ARMC3 | 3.62137E−11 | 1.09 | 0.76 | 0.61 | 0.91 | 87.76 | 80.44 | 7.32 |
| cg23049737 | 1 | RERE; RERE; RERE | 6.3963E−13 | 1.08 | 0.76 | 0.61 | 0.91 | 90.55 | 83.53 | 7.02 |
| cg23073439 | 6 | KDM1B; TPMT; TPMT | 1.39112E−15 | 0.35 | 0.76 | 0.61 | 0.91 | 4.32 | 12.48 | −8.16 |
| cg25802888 | 19 | AURKC; AURKC; AURKC | 3.2021E−11 | 0.90 | 0.76 | 0.61 | 0.91 | 80.52 | 89.11 | −8.60 |
| cg14817541 | 6 | EYA4; EYA4; EYA4 | 2.10205E−12 | 0.54 | 0.76 | 0.61 | 0.91 | 11.35 | 21.04 | −9.69 |
| cg24330922 | 6 | MRPS18B; PPP1 R10 | 1.38236E−18 | 0.45 | 0.76 | 0.61 | 0.91 | 9.09 | 20.31 | −11.21 |
| cg05785516 | 19 | MYADM; MYADM; MYADM; | 4.24173E−25 | 0.40 | 0.76 | 0.61 | 0.91 | 8.94 | 22.22 | −13.27 |
| | | MYADM; MYADM; MYADM; | | | | | | | | |
| | | MYADM; MYADM; MYADM; | | | | | | | | |
| | | MYADM; MYADM; MYADM | | | | | | | | |
| cg04112471 | 3 | SPSB4 | 2.34289E−11 | 1.12 | 0.76 | 0.60 | 0.91 | 81.95 | 73.02 | 8.93 |
| cg05876599 | 22 | PICK1 | 5.01944E−41 | 1.20 | 0.76 | 0.60 | 0.91 | 83.69 | 69.72 | 13.97 |
| cg04289146 | 22 | EIF4ENIF1 | 1.09247E−40 | 1.20 | 0.76 | 0.60 | 0.91 | 80.95 | 67.70 | 13.24 |
| cg25675571 | 20 | ATRN | 1.56076E−40 | 1.19 | 0.76 | 0.60 | 0.91 | 82.13 | 69.22 | 12.91 |
| cg06349093 | 10 | TSPAN14; TSPAN14 | 2.42665E−40 | 1.18 | 0.76 | 0.60 | 0.91 | 81.45 | 68.97 | 12.49 |
| cg01517571 | 1 | ACOT11; FAM151A | 4.30715E−12 | 1.26 | 0.76 | 0.60 | 0.91 | 60.38 | 48.00 | 12.38 |
| cg05113534 | 11 | TECTA | 6.67426E−40 | 1.17 | 0.76 | 0.60 | 0.91 | 81.10 | 69.59 | 11.50 |
| cg04140633 | 1 | IGSF3; IGSF3 | 8.19166E−14 | 1.17 | 0.76 | 0.60 | 0.91 | 75.81 | 64.53 | 11.28 |
| cg03641492 | 6 | SMOC2; SMOC2 | 1.18757E−39 | 1.15 | 0.76 | 0.60 | 0.91 | 83.22 | 72.29 | 10.93 |
| cg12602065 | 9 | LINC00963 | 1.34249E−14 | 1.13 | 0.76 | 0.60 | 0.91 | 83.34 | 73.51 | 9.84 |
| cg24301922 | 17 | SNF8 | 3.4071E−15 | 1.13 | 0.76 | 0.60 | 0.91 | 84.62 | 74.88 | 9.74 |
| cg22184891 | 6 | GPRC6A; GPRC6A; GPRC6A | 1.16539E−12 | 1.13 | 0.76 | 0.60 | 0.91 | 81.95 | 72.50 | 9.46 |
| cg11412298 | 2 | ADAM23 | 1.10059E−12 | 1.12 | 0.76 | 0.60 | 0.91 | 84.27 | 75.40 | 8.87 |
| cg16316127 | 6 | GMDS; GMDS | 1.26323E−12 | 1.11 | 0.76 | 0.60 | 0.91 | 84.99 | 76.33 | 8.65 |
| cg01124275 | 4 | SEPT11 | 3.24069E−14 | 1.11 | 0.76 | 0.60 | 0.91 | 87.50 | 79.05 | 8.45 |
| cg11128249 | 8 | SPIDR; SPIDR; SPIDR; SPIDR | 7.77349E−13 | 1.09 | 0.76 | 0.60 | 0.91 | 88.65 | 81.02 | 7.63 |
| cg01233948 | 15 | TRPM1 | 1.57399E−11 | 1.07 | 0.76 | 0.60 | 0.91 | 92.85 | 87.05 | 5.80 |
| cg10771090 | 1 | TAL1 | 1.05229E−12 | 0.51 | 0.76 | 0.60 | 0.91 | 9.89 | 19.24 | −9.35 |
| cg14935551 | 15 | NR2F2-AS1; NR2F2; NR2F2-AS1; | 5.10832E−14 | 0.48 | 0.76 | 0.60 | 0.91 | 8.48 | 17.83 | −9.35 |
| | | NR2F2-AS1 | | | | | | | | |
| cg03717588 | 11 | LRRC55 | 1.97494E−21 | 0.38 | 0.76 | 0.60 | 0.91 | 6.72 | 17.77 | −11.05 |
| cg23886747 | 11 | ADAMTS8 | 4.41296E−11 | 0.69 | 0.76 | 0.60 | 0.91 | 28.87 | 41.67 | −12.80 |
| cg07064231 | 12 | MDM1 | 5.76539E−42 | 1.37 | 0.75 | 0.60 | 0.91 | 59.08 | 43.15 | 15.93 |
| cg14010852 | 4 | NPFFR2 | 5.30238E−41 | 1.25 | 0.75 | 0.60 | 0.91 | 70.38 | 56.45 | 13.92 |
| cg12395919 | 17 | TMEM49 | 7.44383E−41 | 1.21 | 0.75 | 0.60 | 0.91 | 78.49 | 64.89 | 13.60 |
| cg25583774 | 3 | MIRLET7G; WDR82 | 1.05885E−40 | 1.23 | 0.75 | 0.60 | 0.91 | 71.61 | 58.34 | 13.27 |
| cg02822958 | 2 | ATP6V1E2 | 1.06565E−15 | 1.22 | 0.75 | 0.60 | 0.91 | 71.85 | 59.07 | 12.79 |
| cg12209693 | 7 | GTF2IRD1; GTF2IRD1 | 7.56176E−13 | 1.53 | 0.75 | 0.60 | 0.91 | 34.95 | 22.90 | 12.05 |
| cg07924877 | 11 | NLRX1; NLRX1; NLRX1; NLRX1 | 4.16193E−40 | 1.17 | 0.75 | 0.60 | 0.91 | 81.39 | 69.42 | 11.96 |
| cg17383186 | 1 | NUP210L; NUP210L | 4.0575E−11 | 1.24 | 0.75 | 0.60 | 0.91 | 60.74 | 48.88 | 11.86 |
| cg26477511 | 1 | CYP4X1 | 4.94204E−11 | 1.23 | 0.75 | 0.60 | 0.91 | 62.93 | 51.27 | 11.66 |
| cg17140440 | 2 | SGPP2 | 8.80483E−40 | 1.15 | 0.75 | 0.60 | 0.91 | 83.93 | 72.70 | 11.23 |
| cg22697692 | 3 | HPS3 | 9.20672E−40 | 1.16 | 0.75 | 0.60 | 0.91 | 79.71 | 68.52 | 11.19 |
| cg13319203 | 10 | NEBL; NEBL; NEBL | 1.13843E−13 | 1.16 | 0.75 | 0.60 | 0.91 | 77.97 | 67.19 | 10.78 |
| cg27144309 | 13 | GTF3A | 7.89251E−14 | 1.16 | 0.75 | 0.60 | 0.91 | 78.91 | 68.28 | 10.63 |
| cg04860674 | 3 | SLC9A9 | 1.67344E−39 | 1.14 | 0.75 | 0.60 | 0.91 | 86.19 | 75.59 | 10.59 |
| cg23600177 | 1 | BTBD8 | 5.22302E−15 | 1.15 | 0.75 | 0.60 | 0.91 | 81.48 | 70.98 | 10.50 |
| cg24835999 | 7 | DDC-AS1; DDC; DDC; DDC; DDC; | 2.07148E−13 | 1.14 | 0.75 | 0.60 | 0.91 | 80.56 | 70.47 | 10.09 |
| | | DDC; DDC; DDC | | | | | | | | |
| cg23490161 | 5 | SDHA; CCDC127; SDHA | 4.16935E−39 | 2.51 | 0.75 | 0.60 | 0.91 | 16.09 | 6.42 | 9.67 |
| cg24749077 | 14 | PPP1R3E | 5.10484E−11 | 1.13 | 0.75 | 0.60 | 0.91 | 79.29 | 69.93 | 9.36 |
| cg08670530 | 4 | CCDC96; CCDC96 | 2.73708E−11 | 1.12 | 0.75 | 0.60 | 0.91 | 81.41 | 72.39 | 9.02 |
| cg03549129 | 11 | UBE4A; UBE4A | 3.62163E−13 | 1.11 | 0.75 | 0.60 | 0.91 | 86.03 | 77.48 | 8.55 |
| cg25103393 | 5 | RASGRF2 | 1.89497E−12 | 1.11 | 0.75 | 0.60 | 0.91 | 85.38 | 76.91 | 8.48 |
| cg10218661 | 13 | CLYBL; CLYBL; LOC101927437 | 1.56492E−12 | 1.11 | 0.75 | 0.60 | 0.91 | 85.75 | 77.35 | 8.40 |
| cg19092300 | 19 | ZNF254; ZNF254; ZNF254 | 5.87363E−11 | 1.11 | 0.75 | 0.60 | 0.91 | 84.07 | 75.82 | 8.24 |
| cg13322601 | 4 | ODZ3 | 4.66417E−14 | 1.10 | 0.75 | 0.60 | 0.91 | 88.07 | 79.87 | 8.21 |
| cg21633963 | 2 | DTNB; DTNB; DTNB; DTNB; | 1.92107E−13 | 1.10 | 0.75 | 0.60 | 0.91 | 87.50 | 79.30 | 8.20 |
| | | DTNB; DTNB; DTNB; DTNB | | | | | | | | |
| cg19503700 | 19 | SIGLEC10; SIGLEC10; SIGLEC10; | 2.09243E−38 | 1.10 | 0.75 | 0.60 | 0.91 | 91.66 | 83.67 | 7.99 |
| | | SIGLEC10; SIGLEC10; SIGLEC10; | | | | | | | | |
| | | SIGLEC10; LOC100129083 | | | | | | | | |
| cg02325160 | 4 | FAT1 | 1.18422E−11 | 1.10 | 0.75 | 0.60 | 0.91 | 86.50 | 78.64 | 7.87 |
| cg20778669 | 10 | MGMT | 4.33792E−11 | 1.10 | 0.75 | 0.60 | 0.91 | 86.10 | 78.33 | 7.76 |
| cg22599978 | 8 | R3HCC1; R3HCC1; R3HCC1 | 6.40512E−12 | 1.10 | 0.75 | 0.60 | 0.91 | 87.49 | 79.81 | 7.68 |
| cg14421180 | 9 | FNBP1 | 5.13934E−20 | 0.24 | 0.75 | 0.60 | 0.91 | 2.76 | 11.36 | −8.60 |
| cg23987493 | 2 | LOC100996579; LOC100996579; | 5.4435E−21 | 0.72 | 0.75 | 0.60 | 0.91 | 48.52 | 67.19 | −18.68 |
| | | LOC101929512 | | | | | | | | |
| cg06566419 | 10 | LCOR | 4.62509E−41 | 1.22 | 0.75 | 0.59 | 0.91 | 79.36 | 65.31 | 14.05 |
| cg08250541 | 8 | MTFR1; MTFR1; MTFR1 | 2.40876E−15 | 1.27 | 0.75 | 0.59 | 0.91 | 63.55 | 49.88 | 13.67 |
| cg00328411 | 4 | NR3C2; NR3C2 | 1.91006E−40 | 1.18 | 0.75 | 0.59 | 0.91 | 83.89 | 71.18 | 12.71 |
| cg04683436 | 15 | HDGFRP3 | 3.12274E−13 | 1.23 | 0.75 | 0.59 | 0.91 | 65.90 | 53.42 | 12.48 |
| cg12870014 | 12 | ANKRD13A | 6.53947E−13 | 1.23 | 0.75 | 0.59 | 0.91 | 65.32 | 52.94 | 12.39 |
| cg12936709 | 17 | OR1D2 | 2.54704E−12 | 1.45 | 0.75 | 0.59 | 0.91 | 39.66 | 27.40 | 12.27 |
| cg16112303 | 2 | SULT1C4 | 4.26128E−40 | 1.18 | 0.75 | 0.59 | 0.91 | 77.69 | 65.75 | 11.94 |
| cg17734022 | 5 | C5orf67 | 5.32813E−40 | 1.16 | 0.75 | 0.59 | 0.91 | 82.84 | 71.11 | 11.72 |
| cg14137316 | 17 | BCAS3; BCAS3 | 1.42014E−11 | 1.19 | 0.75 | 0.59 | 0.91 | 70.10 | 58.91 | 11.19 |
| cg24407878 | 9 | HNRNPK; HNRNPK; HNRNPK; | 4.96572E−11 | 1.18 | 0.75 | 0.59 | 0.91 | 70.94 | 60.14 | 10.80 |
| | | HNRNPK; HNRNPK; HNRNPK | | | | | | | | |
| cg05476330 | 5 | DNAJC18; DNAJC18 | 4.4033E−12 | 1.17 | 0.75 | 0.59 | 0.91 | 75.15 | 64.50 | 10.65 |
| cg11773859 | 5 | MEF2C; MEF2C; MEF2C; MEF2C; | 6.06469E−15 | 1.14 | 0.75 | 0.59 | 0.91 | 82.83 | 72.74 | 10.09 |
| | | MEF2C; MEF2C; MEF2C | | | | | | | | |
| cg11815438 | 6 | TNXB | 2.84551E−39 | 1.13 | 0.75 | 0.59 | 0.91 | 86.05 | 76.00 | 10.06 |
| cg07298482 | 7 | SUNC1; SUNC1 | 4.45966E−11 | 1.15 | 0.75 | 0.59 | 0.91 | 76.35 | 66.39 | 9.96 |
| cg03614649 | 16 | AFG3L1; CENPBD1; AFG3L1; | 3.49955E−39 | 1.13 | 0.75 | 0.59 | 0.91 | 86.54 | 76.69 | 9.85 |
| | | AFG3L1 | | | | | | | | |
| cg19058507 | 12 | DYRK4; DYRK4; DYRK4 | 9.40864E−14 | 1.14 | 0.75 | 0.59 | 0.91 | 82.22 | 72.41 | 9.81 |
| cg09894683 | 8 | PSD3; PSD3 | 1.04285E−11 | 1.14 | 0.75 | 0.59 | 0.91 | 79.29 | 69.62 | 9.67 |
| cg09305096 | 2 | GULP1 | 1.66537E−11 | 1.14 | 0.75 | 0.59 | 0.91 | 79.17 | 69.56 | 9.61 |
| cg00940140 | 20 | GNAS; GNAS; GNAS; GNAS; | 2.82445E−12 | 1.13 | 0.75 | 0.59 | 0.91 | 82.50 | 73.33 | 9.17 |
| | | GNAS; GNAS; GNAS; GNAS | | | | | | | | |
| cg12237926 | 15 | SEMA6D | 1.81454E−13 | 1.12 | 0.75 | 0.59 | 0.91 | 84.27 | 75.11 | 9.16 |
| cg00386405 | 19 | ZNF536 | 4.74585E−15 | 1.11 | 0.75 | 0.59 | 0.91 | 87.39 | 78.55 | 8.84 |
| cg16870548 | 22 | NPTXR | 7.57383E−13 | 1.11 | 0.75 | 0.59 | 0.91 | 86.43 | 78.11 | 8.32 |
| cg24364004 | 16 | NKD1 | 8.67892E−12 | 1.10 | 0.75 | 0.59 | 0.91 | 86.63 | 78.76 | 7.88 |
| cg16672904 | 8 | TSNARE1 | 2.83467E−11 | 1.10 | 0.75 | 0.59 | 0.91 | 86.07 | 78.22 | 7.84 |
| cg20800039 | 15 | ADAM 10 | 9.85649E−12 | 1.06 | 0.75 | 0.59 | 0.91 | 93.45 | 87.80 | 5.65 |
| cg06891548 | 1 | GGPS1; ARID4B; GGPS1; GGPS1; | 1.57038E−13 | 0.39 | 0.75 | 0.59 | 0.91 | 5.09 | 12.94 | −7.85 |
| | | ARID4B | | | | | | | | |
| cg09276368 | 10 | SLIT1 | 2.35561E−15 | 0.33 | 0.75 | 0.59 | 0.91 | 3.89 | 11.79 | −7.89 |
| cg16605745 | 12 | RPAP3; RPAP3; RPAP3; RPAP3; | 8.3944E−15 | 0.52 | 0.75 | 0.59 | 0.91 | 11.91 | 22.78 | −10.88 |
| | | RPAP3; RPAP3 | | | | | | | | |
| cg19183433 | 2 | CWC22 | 3.3185E−14 | 0.31 | 0.75 | 0.59 | 0.91 | 3.21 | 10.46 | −7.25 |
| cg05432848 | 8 | GDAP1; GDAP1 | 2.7823E−15 | 0.47 | 0.75 | 0.59 | 0.91 | 8.95 | 18.94 | −9.99 |
| cg06255442 | 7 | NDUFA5 | 6.45503E−42 | 1.32 | 0.75 | 0.59 | 0.91 | 64.83 | 49.00 | 15.83 |
| cg10521230 | 3 | DZIP1L | 1.33732E−41 | 1.32 | 0.75 | 0.59 | 0.91 | 62.44 | 47.26 | 15.18 |
| cg14224760 | 8 | CNGB3 | 6.04943E−41 | 1.24 | 0.75 | 0.59 | 0.91 | 71.74 | 57.94 | 13.80 |
| cg20489040 | 22 | MAPK1; MAPK1 | 3.23975E−14 | 1.22 | 0.75 | 0.59 | 0.91 | 69.62 | 57.16 | 12.47 |
| cg14459203 | 11 | CCDC34; CCDC34 | 1.28431E−13 | 1.19 | 0.75 | 0.59 | 0.91 | 73.23 | 61.58 | 11.66 |
| cg05727299 | 2 | XRCC5 | 7.99644E−40 | 1.16 | 0.75 | 0.59 | 0.91 | 80.95 | 69.62 | 11.33 |
| cg15841349 | 12 | GLT1D1 | 1.07815E−39 | 1.15 | 0.75 | 0.59 | 0.91 | 82.61 | 71.58 | 11.03 |
| cg19088553 | 6 | GRIK2; GRIK2; GRIK2 | 1.35618E−14 | 1.16 | 0.75 | 0.59 | 0.91 | 78.80 | 67.85 | 10.95 |
| cg11886750 | 10 | ADK; ADK; ADK; ADK | 1.80918E−11 | 1.17 | 0.75 | 0.59 | 0.91 | 72.98 | 62.27 | 10.72 |
| cg12171458 | 12 | CBX5 | 2.46952E−12 | 1.16 | 0.75 | 0.59 | 0.91 | 76.53 | 66.02 | 10.50 |
| cg03474251 | 11 | ALG9; ALG9; ALG9; ALG9 | 5.24477E−14 | 1.15 | 0.75 | 0.59 | 0.91 | 80.24 | 69.83 | 10.40 |
| cg08013260 | 12 | NAA25 | 1.39885E−14 | 1.15 | 0.75 | 0.59 | 0.91 | 81.26 | 70.90 | 10.35 |
| cg11925907 | 8 | CSMD1 | 1.81075E−14 | 1.14 | 0.75 | 0.59 | 0.91 | 82.01 | 71.87 | 10.14 |
| cg06338841 | 14 | MNAT1; MNAT1 | 4.12916E−39 | 1.13 | 0.75 | 0.59 | 0.91 | 84.91 | 75.23 | 9.68 |
| cg11860760 | 5 | RNF130 | 1.54307E−12 | 1.13 | 0.75 | 0.59 | 0.91 | 81.41 | 71.87 | 9.54 |
| cg01999051 | 6 | BRP44L | 1.33212E−11 | 1.13 | 0.75 | 0.59 | 0.91 | 80.31 | 70.91 | 9.40 |
| cg12944311 | 1 | PEX14 | 3.54121E−13 | 1.12 | 0.75 | 0.59 | 0.91 | 84.41 | 75.39 | 9.01 |
| cg14207163 | 1 | C1orf51 | 2.09209E−12 | 1.12 | 0.75 | 0.59 | 0.91 | 84.33 | 75.58 | 8.75 |
| cg26673903 | 7 | WNT16; WNT16 | 9.37625E−13 | 1.11 | 0.75 | 0.59 | 0.91 | 84.89 | 76.17 | 8.72 |
| cg05020535 | 10 | TCERG1L | 5.81229E−12 | 1.11 | 0.75 | 0.59 | 0.91 | 84.39 | 75.83 | 8.56 |
| cg19385365 | 7 | DGKI | 7.09382E−12 | 1.11 | 0.75 | 0.59 | 0.91 | 84.66 | 76.20 | 8.46 |
| cg06958766 | 14 | TGFB3 | 4.3182E−12 | 1.11 | 0.75 | 0.59 | 0.91 | 85.86 | 77.65 | 8.21 |
| cg11210410 | 9 | WDR34; MIR1268A | 3.27412E−11 | 1.10 | 0.75 | 0.59 | 0.91 | 86.10 | 78.29 | 7.81 |
| cg08500179 | 17 | TOM1L2; TOM1L2; TOM1L2; | 3.97008E−13 | 1.09 | 0.75 | 0.59 | 0.91 | 88.74 | 81.05 | 7.70 |
| | | TOM1L2; TOM1L2; TOM1L2 | | | | | | | | |
| cg10756110 | 2 | RAPGEF4; RAPGEF4-AS1 | 4.73103E−11 | 1.08 | 0.75 | 0.59 | 0.91 | 90.17 | 83.62 | 6.55 |
| cg11112162 | 1 | S100A11 | 4.86096E−12 | 0.28 | 0.75 | 0.59 | 0.91 | 2.38 | 8.62 | −6.23 |
| ch.8.2465681F | 8 | FAM91A1 | 1.84156E−11 | 0.49 | 0.75 | 0.59 | 0.91 | 7.69 | 15.73 | −8.04 |
| cg19221251 | 7 | KMT2E-AS1; KMT2E; KMT2E | 2.5873E−15 | 0.35 | 0.75 | 0.59 | 0.91 | 4.40 | 12.51 | −8.11 |
| cg07849302 | 10 | TCF7L2; TCF7L2; TCF7L2; | 2.53557E−14 | 0.46 | 0.75 | 0.59 | 0.91 | 7.89 | 17.12 | −9.23 |
| | | TCF7L2; TCF7L2; TCF7L2 | | | | | | | | |
| cg09970125 | 8 | ADAM3A | 3.21501E−41 | 1.23 | 0.74 | 0.58 | 0.90 | 76.67 | 62.29 | 14.38 |
| cg05439262 | 13 | PAN3 | 6.10899E−41 | 1.22 | 0.74 | 0.58 | 0.90 | 76.17 | 62.38 | 13.79 |
| cg08717144 | 9 | ELAVL2 | 8.12483E−41 | 1.20 | 0.74 | 0.58 | 0.90 | 80.86 | 67.34 | 13.52 |
| cg07394978 | 7 | TRG-AS1 | 8.43292E−41 | 1.21 | 0.74 | 0.58 | 0.90 | 78.15 | 64.66 | 13.49 |
| cg22764861 | 8 | MCPH1; MCPH1; MCPH1 | 1.69594E−15 | 1.25 | 0.74 | 0.58 | 0.90 | 66.26 | 52.81 | 13.46 |
| cg14270998 | 7 | EXOC4 | 1.91903E−40 | 1.21 | 0.74 | 0.58 | 0.90 | 74.08 | 61.37 | 12.71 |
| cg21859762 | 3 | PIK3CB; PIK3CB | 2.64373E−40 | 1.18 | 0.74 | 0.58 | 0.90 | 79.57 | 67.16 | 12.40 |
| cg09120609 | 7 | CADPS2; CADPS2; CADPS2 | 2.08168E−13 | 1.20 | 0.74 | 0.58 | 0.90 | 71.94 | 60.17 | 11.77 |
| cg14613546 | 9 | BNC2 | 5.17547E−40 | 1.17 | 0.74 | 0.58 | 0.90 | 79.62 | 67.87 | 11.75 |
| cg04184744 | 14 | AP4S1 | 3.28193E−15 | 1.18 | 0.74 | 0.58 | 0.90 | 76.31 | 64.56 | 11.75 |
| cg19469447 | 10 | CYP2E1 | 7.08213E−40 | 2.07 | 0.74 | 0.58 | 0.90 | 22.18 | 10.74 | 11.45 |
| cg15935723 | 20 | LOC100130264; SLC24A3 | 3.57627E−12 | 1.55 | 0.74 | 0.58 | 0.90 | 32.17 | 20.75 | 11.42 |
| cg12626639 | 10 | GOT1 | 9.25417E−40 | 1.16 | 0.74 | 0.58 | 0.90 | 82.72 | 71.54 | 11.18 |
| cg04229882 | 4 | SLBP; SLBP; SLBP | 1.24888E−39 | 1.14 | 0.74 | 0.58 | 0.90 | 87.21 | 76.32 | 10.88 |
| cg21323406 | 9 | FKTN; FKTN; FKTN | 2.36098E−12 | 1.16 | 0.74 | 0.58 | 0.90 | 77.26 | 66.89 | 10.37 |
| cg24517672 | 2 | ITGA6; ITGA6 | 3.22362E−13 | 1.14 | 0.74 | 0.58 | 0.90 | 80.25 | 70.17 | 10.09 |
| cg12782542 | 12 | SRGAP1 | 4.58464E−11 | 1.14 | 0.74 | 0.58 | 0.90 | 77.76 | 68.07 | 9.69 |
| cg16962473 | 7 | FAM221A; FAM221A; FAM221A; | 2.40664E−11 | 1.14 | 0.74 | 0.58 | 0.90 | 78.75 | 69.12 | 9.62 |
| | | FAM221A | | | | | | | | |
| cg14044392 | 9 | NTNG2 | 1.05325E−13 | 1.13 | 0.74 | 0.58 | 0.90 | 83.06 | 73.49 | 9.58 |
| cg08750053 | 22 | DMC1 | 5.44252E−12 | 1.13 | 0.74 | 0.58 | 0.90 | 80.74 | 71.27 | 9.47 |
| cg19879189 | 6 | TNXB | 1.38737E−12 | 1.13 | 0.74 | 0.58 | 0.90 | 81.80 | 72.33 | 9.47 |
| cg08902519 | 8 | SDR16C5 | 1.72251E−11 | 1.12 | 0.74 | 0.58 | 0.90 | 81.67 | 72.62 | 9.05 |
| cg27359469 | 6 | C6orf192 | 3.04923E−11 | 1.12 | 0.74 | 0.58 | 0.90 | 82.01 | 73.15 | 8.86 |
| cg03794759 | 5 | SPOCK1 | 9.82783E−12 | 1.12 | 0.74 | 0.58 | 0.90 | 83.73 | 75.09 | 8.64 |
| cg12759597 | 1 | MTX1; MTX1 | 1.08429E−13 | 1.11 | 0.74 | 0.58 | 0.90 | 86.73 | 78.21 | 8.52 |
| cg26880297 | 1 | SKI | 3.18969E−12 | 1.11 | 0.74 | 0.58 | 0.90 | 85.00 | 76.50 | 8.50 |
| cg07192243 | 13 | COG6; COG6; COG6 | 1.18835E−13 | 1.10 | 0.74 | 0.58 | 0.90 | 87.58 | 79.34 | 8.24 |
| cg03068949 | 12 | DCTN2; DCTN2; DCTN2 | 1.29018E−13 | 1.10 | 0.74 | 0.58 | 0.90 | 87.90 | 79.77 | 8.13 |
| cg13410153 | 5 | LOC101929710 | 1.06522E−11 | 1.10 | 0.74 | 0.58 | 0.90 | 85.87 | 77.82 | 8.06 |
| cg18444028 | 3 | GPR160 | 5.63641E−11 | 1.09 | 0.74 | 0.58 | 0.90 | 86.85 | 79.34 | 7.51 |
| cg03601985 | 1 | C1orf115 | 7.65746E−13 | 1.09 | 0.74 | 0.58 | 0.90 | 89.15 | 81.68 | 7.47 |
| cg10919176 | 8 | NUDCD1; ENY2 | 1.00754E−11 | 0.44 | 0.74 | 0.58 | 0.90 | 5.96 | 13.49 | −7.53 |
| cg09123433 | 6 | HIST1H4H | 3.89129E−13 | 0.42 | 0.74 | 0.58 | 0.90 | 5.67 | 13.63 | −7.95 |
| cg10138522 | 2 | INO80D | 9.86487E−14 | 0.50 | 0.74 | 0.58 | 0.90 | 9.44 | 19.03 | −9.59 |
| cg24917037 | 9 | NXNL2; NXNL2 | 4.8822E−11 | 0.61 | 0.74 | 0.58 | 0.90 | 15.95 | 26.25 | −10.31 |
| cg22608385 | 4 | SYNPO2; SYNPO2; SYNPO2; | 3.04948E−12 | 0.61 | 0.74 | 0.58 | 0.90 | 17.49 | 28.84 | −11.36 |
| | | SYNPO2; SYNPO2 | | | | | | | | |
| cg19617678 | 1 | FOXJ3; FOXJ3; FOXJ3; FOXJ3 | 4.78562E−24 | 0.59 | 0.74 | 0.58 | 0.90 | 27.11 | 46.02 | −18.91 |
| cg15985376 | 11 | B3GAT3; B3GAT3; B3GAT3; | 3.5179E−15 | 0.29 | 0.74 | 0.58 | 0.90 | 3.02 | 10.46 | −7.44 |
| | | B3GAT3; B3GAT3 | | | | | | | | |
| cg13440846 | 1 | C1orf97 | 2.3408E−14 | 1.21 | 0.74 | 0.58 | 0.90 | 70.70 | 58.32 | 12.38 |
| cg05149988 | 3 | ATP13A3 | 1.95005E−12 | 1.19 | 0.74 | 0.58 | 0.90 | 72.01 | 60.68 | 11.33 |
| cg21763634 | 1 | RABGAP1L; RABGAP1L; | 1.25141E−39 | 1.15 | 0.74 | 0.58 | 0.90 | 81.72 | 70.84 | 10.88 |
| | | RABGAP1L; RABGAP1L | | | | | | | | |
| cg22988458 | 7 | TRRAP | 2.16087E−39 | 1.13 | 0.74 | 0.58 | 0.90 | 87.55 | 77.21 | 10.34 |
| cg17633639 | 3 | DNAJC13 | 2.85837E−15 | 1.14 | 0.74 | 0.58 | 0.90 | 83.10 | 72.88 | 10.22 |
| cg11589832 | 10 | ARID5B | 2.88752E−14 | 1.13 | 0.74 | 0.58 | 0.90 | 83.64 | 74.03 | 9.62 |
| cg22100985 | 19 | PRPF31 | 2.40586E−11 | 1.13 | 0.74 | 0.58 | 0.90 | 81.29 | 72.21 | 9.07 |
| cg14415283 | 9 | PPP2R4; PPP2R4; PPP2R4; PPP2R4 | 5.1025E−13 | 1.11 | 0.74 | 0.58 | 0.90 | 86.08 | 77.60 | 8.48 |
| cg26346892 | 1 | PRRC2C | 4.33473E−14 | 1.10 | 0.74 | 0.58 | 0.90 | 88.61 | 80.58 | 8.04 |
| cg09061733 | 11 | SERPING1; SERPING1 | 6.30813E−12 | 1.10 | 0.74 | 0.58 | 0.90 | 87.21 | 79.46 | 7.76 |
| cg20216982 | 2 | HDAC4 | 3.36478E−12 | 1.08 | 0.74 | 0.58 | 0.90 | 91.45 | 84.95 | 6.49 |
| cg21326516 | 3 | KCNMB2; KCNMB2-AS1 | 1.69694E−41 | 1.27 | 0.74 | 0.58 | 0.90 | 71.14 | 56.18 | 14.97 |
| cg00597949 | 10 | SVIL | 2.91223E−41 | 1.28 | 0.74 | 0.58 | 0.90 | 66.84 | 52.36 | 14.47 |
| cg07764972 | 4 | GUCY1A3 | 7.98193E−41 | 1.21 | 0.74 | 0.58 | 0.90 | 77.85 | 64.32 | 13.54 |
| cg18399457 | 6 | LSM2 | 1.3775E−40 | 1.20 | 0.74 | 0.58 | 0.90 | 76.65 | 63.62 | 13.03 |
| cg24078669 | 16 | EEF2K | 1.02758E−13 | 1.24 | 0.74 | 0.58 | 0.90 | 65.49 | 52.74 | 12.74 |
| cg14618310 | 10 | C10orf84; C10orf84 | 7.26721E−40 | 1.17 | 0.74 | 0.58 | 0.90 | 80.12 | 68.70 | 11.42 |
| cg14783915 | 6 | GMDS; GMDS | 8.77989E−40 | 1.16 | 0.74 | 0.58 | 0.90 | 79.70 | 68.47 | 11.23 |
| cg17468543 | 8 | ASAP1 | 1.44211E−39 | 1.15 | 0.74 | 0.58 | 0.90 | 84.17 | 73.43 | 10.74 |
| cg17085976 | 3 | TMEM212-AS1 | 1.92117E−39 | 1.14 | 0.74 | 0.58 | 0.90 | 85.27 | 74.81 | 10.45 |
| cg23935289 | 14 | AKAP6 | 3.20071E−15 | 1.13 | 0.74 | 0.58 | 0.90 | 83.57 | 73.66 | 9.91 |
| cg02336095 | 22 | MIR6817; CRYBB2P1; | 6.31851E−13 | 1.12 | 0.74 | 0.58 | 0.90 | 83.35 | 74.15 | 9.20 |
| | | CRYBB2P1 | | | | | | | | |
| cg17303577 | 8 | RIMS2; RIMS2; RIMS2; RIMS2; | 2.82569E−12 | 1.12 | 0.74 | 0.58 | 0.90 | 83.35 | 74.40 | 8.95 |
| | | RIMS2 | | | | | | | | |
| cg11006283 | 6 | TIAM2 | 4.21063E−11 | 1.11 | 0.74 | 0.58 | 0.90 | 83.28 | 74.78 | 8.50 |
| cg25019898 | 10 | WDR37 | 8.41425E−12 | 1.11 | 0.74 | 0.58 | 0.90 | 85.70 | 77.55 | 8.15 |
| cg13112267 | 8 | TSNARE1; TSNARE1 | 2.6444E−14 | 1.10 | 0.74 | 0.58 | 0.90 | 89.27 | 81.39 | 7.88 |
| cg26978172 | 6 | DDAH2 | 7.68719E−14 | 1.06 | 0.74 | 0.58 | 0.90 | 95.65 | 90.36 | 5.30 |
| cg16969189 | 7 | IKZF1; IKZF1; IKZF1; IKZF1; | 9.16703E−12 | 0.47 | 0.74 | 0.58 | 0.90 | 6.97 | 14.88 | −7.91 |
| | | IKZF1; IKZF1; IKZF1; IKZF1 | | | | | | | | |
| cg10064897 | 1 | PIK3R3; PIK3R3 | 1.18234E−11 | 0.59 | 0.74 | 0.58 | 0.90 | 14.49 | 24.73 | −10.25 |
| cg05003157 | 1 | ZBTB41; CRB1 | 1.71594E−15 | 0.53 | 0.74 | 0.58 | 0.90 | 12.98 | 24.50 | −11.52 |
| cg07614835 | 19 | SBNO2 | 7.306E−14 | 0.61 | 0.74 | 0.58 | 0.90 | 19.36 | 31.99 | −12.63 |
| cg06242416 | 16 | METTL9 | 2.94991E−41 | 1.29 | 0.74 | 0.58 | 0.90 | 65.03 | 50.57 | 14.46 |
| cg07878960 | 15 | HERC1 | 5.61049E−41 | 1.23 | 0.74 | 0.58 | 0.90 | 74.75 | 60.89 | 13.87 |
| cg06133339 | 9 | FAM163B | 6.58122E−41 | 1.21 | 0.74 | 0.58 | 0.90 | 78.15 | 64.43 | 13.72 |
| cg17871838 | 6 | MIR1913; RPS6KA2 | 7.59855E−41 | 1.21 | 0.74 | 0.58 | 0.90 | 77.96 | 64.37 | 13.59 |
| cg24775884 | 2 | ICA1L | 8.33108E−41 | 1.24 | 0.74 | 0.58 | 0.90 | 70.18 | 56.68 | 13.50 |
| cg14865086 | 17 | COPS3 | 1.00097E−40 | 1.23 | 0.74 | 0.58 | 0.90 | 72.47 | 59.14 | 13.33 |
| cg00564790 | 8 | POLB | 5.49392E−14 | 1.25 | 0.74 | 0.58 | 0.90 | 64.40 | 51.43 | 12.97 |
| cg02370442 | 1 | ZMYND12; ZMYND12 | 5.9546E−15 | 1.22 | 0.74 | 0.58 | 0.90 | 70.22 | 57.53 | 12.69 |
| cg12060669 | 8 | LOXL2 | 1.70641E−14 | 1.61 | 0.74 | 0.58 | 0.90 | 33.09 | 20.60 | 12.49 |
| cg10632760 | 6 | LYRM2; LOC101929057 | 1.42722E−13 | 1.22 | 0.74 | 0.58 | 0.90 | 68.43 | 56.09 | 12.34 |
| cg12292595 | 2 | NIF3L1; NIF3L1; NIF3L1; NIF3L1 | 1.47237E−15 | 1.19 | 0.74 | 0.58 | 0.90 | 75.87 | 63.91 | 11.96 |
| cg19426761 | 4 | MIR577; UGT8; UGT8 | 9.50823E−15 | 1.19 | 0.74 | 0.58 | 0.90 | 74.36 | 62.43 | 11.93 |
| cg06530987 | 4 | TMEM184C | 1.37766E−11 | 1.22 | 0.74 | 0.58 | 0.90 | 65.46 | 53.73 | 11.73 |
| cg08124030 | 3 | TM4SF1 | 6.75973E−13 | 1.20 | 0.74 | 0.58 | 0.90 | 70.89 | 59.19 | 11.70 |
| cg13472672 | 4 | RELL1; RELL1 | 7.05934E−15 | 1.18 | 0.74 | 0.58 | 0.90 | 76.26 | 64.65 | 11.62 |
| cg18080470 | 10 | PTPRE; PTPRE | 5.27639E−11 | 1.20 | 0.74 | 0.58 | 0.90 | 66.57 | 55.26 | 11.31 |
| cg20338182 | 7 | HEPACAM2 | 1.01369E−39 | 1.16 | 0.74 | 0.58 | 0.90 | 81.97 | 70.87 | 11.09 |
| cg24091611 | 10 | GLUD1 | 1.03795E−39 | 1.15 | 0.74 | 0.58 | 0.90 | 84.02 | 72.95 | 11.07 |
| cg08320351 | 6 | CASC15 | 1.00152E−13 | 1.17 | 0.74 | 0.58 | 0.90 | 76.87 | 65.84 | 11.03 |
| cg17675311 | 13 | TSC22D1 | 7.62745E−14 | 1.16 | 0.74 | 0.58 | 0.90 | 78.09 | 67.27 | 10.82 |
| cg19563510 | 17 | MAFG; MAFG | 1.0855E−11 | 1.59 | 0.74 | 0.58 | 0.90 | 29.14 | 18.35 | 10.80 |
| cg22073794 | 1 | MAST2 | 1.4324E−39 | 1.15 | 0.74 | 0.58 | 0.90 | 83.93 | 73.18 | 10.75 |
| cg20576322 | 4 | WDFY3 | 3.41246E−12 | 1.16 | 0.74 | 0.58 | 0.90 | 76.42 | 65.96 | 10.46 |
| cg03624321 | 3 | EOGT; EOGT; EOGT | 1.09962E−13 | 1.15 | 0.74 | 0.58 | 0.90 | 79.70 | 69.29 | 10.40 |
| cg15418738 | 4 | KLHL5; KLHL5; KLHL5; KLHL5 | 3.85865E−12 | 1.15 | 0.74 | 0.58 | 0.90 | 77.27 | 67.00 | 10.27 |
| cg27351543 | 16 | SNX29 | 3.12521E−15 | 1.13 | 0.74 | 0.58 | 0.90 | 83.92 | 73.94 | 9.98 |
| cg25094834 | 6 | NUP153 | 3.02639E−13 | 1.13 | 0.74 | 0.58 | 0.90 | 82.64 | 73.13 | 9.51 |
| cg06223834 | 16 | ADCY9 | 2.09483E−11 | 1.13 | 0.74 | 0.58 | 0.90 | 80.18 | 70.83 | 9.35 |
| cg00506540 | 4 | HAUS3 | 3.73972E−14 | 1.11 | 0.74 | 0.58 | 0.90 | 86.13 | 77.27 | 8.86 |
| cg02116955 | 8 | NAT1; NAT1; NAT1; NAT1; NAT1; | 1.21711E−12 | 1.12 | 0.74 | 0.58 | 0.90 | 84.52 | 75.73 | 8.79 |
| | | NAT1; NAT1; NAT1; NAT1 | | | | | | | | |
| cg08288559 | 5 | RAD17; RAD17; RAD17; RAD17; | 3.42482E−13 | 1.11 | 0.74 | 0.58 | 0.90 | 86.03 | 77.47 | 8.56 |
| | | RAD17; RAD17; RAD17; RAD17; | | | | | | | | |
| | | RAD17 | | | | | | | | |
| cg21015054 | 4 | ELOVL6; ELOVL6 | 2.9665E−11 | 1.11 | 0.74 | 0.58 | 0.90 | 83.87 | 75.45 | 8.42 |
| cg16371865 | 8 | TRAPPC9; TRAPPC9 | 7.22224E−12 | 1.11 | 0.74 | 0.58 | 0.90 | 85.20 | 76.89 | 8.31 |
| cg21004032 | 17 | NUP85; NUP85 | 1.71953E−38 | 1.10 | 0.74 | 0.58 | 0.90 | 89.77 | 81.57 | 8.20 |
| cg07419396 | 4 | ZFYVE28 | 3.85255E−38 | 1.09 | 0.74 | 0.58 | 0.90 | 91.52 | 84.18 | 7.34 |
| cg09756257 | 16 | UBE2I; UBE2I; UBE2I; UBE2I | 4.90988E−12 | 1.08 | 0.74 | 0.58 | 0.90 | 89.85 | 82.88 | 6.98 |
| cg13782795 | 2 | VSNL1 | 1.89732E−11 | 1.08 | 0.74 | 0.58 | 0.90 | 90.96 | 84.53 | 6.43 |
| cg05236768 | 16 | NAT15; NAT15; NAT15 | 7.87826E−13 | 1.07 | 0.74 | 0.58 | 0.90 | 92.64 | 86.40 | 6.25 |
| cg02898500 | 13 | PROZ | 5.17978E−12 | 1.06 | 0.74 | 0.58 | 0.90 | 94.09 | 88.60 | 5.48 |
| cg09171882 | 12 | GLIPR1 | 1.45057E−11 | 0.45 | 0.74 | 0.58 | 0.90 | 6.03 | 13.51 | −7.49 |
| cg10929629 | 13 | RNF219 | 8.44916E−15 | 0.46 | 0.74 | 0.58 | 0.90 | 7.99 | 17.43 | −9.44 |
| cg05154615 | 18 | SETBP1; SETBP1 | 3.8645E−19 | 0.54 | 0.74 | 0.58 | 0.90 | 16.86 | 31.13 | −14.27 |
| cg11862389 | 1 | USH2A; USH2A | 1.82695E−13 | 1.22 | 0.73 | 0.57 | 0.90 | 68.09 | 55.75 | 12.34 |
| cg07495626 | 17 | TRPV1 | 9.23585E−15 | 1.19 | 0.73 | 0.57 | 0.90 | 73.99 | 61.98 | 12.01 |
| cg04267605 | 4 | LRRC66 | 4.1576E−40 | 1.19 | 0.73 | 0.57 | 0.90 | 76.60 | 64.63 | 11.97 |
| cg11224705 | 16 | CPPED1; CPPED1 | 4.76714E−40 | 1.17 | 0.73 | 0.57 | 0.90 | 81.74 | 69.91 | 11.83 |
| cg17162166 | 14 | C14orf142; UBR7; UBR7 | 4.82862E−40 | 1.18 | 0.73 | 0.57 | 0.90 | 77.44 | 65.62 | 11.82 |
| cg11295173 | 1 | UCHL5; UCHL5; UCHL5; UCHL5; | 9.97807E−15 | 1.19 | 0.73 | 0.57 | 0.90 | 75.02 | 63.22 | 11.80 |
| | | UCHL5; UCHL5; UCHL5; UCHL5; | | | | | | | | |
| | | UCHL5; UCHL5 | | | | | | | | |
| cg17684478 | 11 | FOXR1 | 2.58599E−14 | 1.19 | 0.73 | 0.57 | 0.90 | 74.42 | 62.68 | 11.74 |
| cg23138027 | 11 | CCS | 7.09476E−40 | 1.16 | 0.73 | 0.57 | 0.90 | 81.93 | 70.49 | 11.44 |
| cg26770470 | 2 | GRB14; GRB14 | 8.18133E−40 | 1.17 | 0.73 | 0.57 | 0.90 | 79.42 | 68.11 | 11.30 |
| cg05489292 | 12 | MAPKAPK5; MAPKAPK5 | 8.46625E−40 | 1.16 | 0.73 | 0.57 | 0.90 | 79.91 | 68.64 | 11.27 |
| cg07964527 | 15 | PDE8A; PDE8A | 1.80068E−13 | 1.17 | 0.73 | 0.57 | 0.90 | 75.55 | 64.37 | 11.18 |
| cg16619557 | 9 | WDR5; WDR5 | 1.00176E−13 | 1.17 | 0.73 | 0.57 | 0.90 | 76.23 | 65.07 | 11.16 |
| cg08792074 | 4 | COL25A1; COL25A1; COL25A1; | 1.12208E−14 | 1.16 | 0.73 | 0.57 | 0.90 | 78.96 | 67.99 | 10.97 |
| | | COL25A1 | | | | | | | | |
| cg13546321 | 12 | CUX2 | 4.72016E−11 | 1.17 | 0.73 | 0.57 | 0.90 | 72.89 | 62.36 | 10.53 |
| cg06168235 | 18 | PIK3C3 | 2.37744E−12 | 1.15 | 0.73 | 0.57 | 0.90 | 78.36 | 68.21 | 10.15 |
| cg19513064 | 15 | SCG5; SCG5 | 4.02137E−12 | 1.15 | 0.73 | 0.57 | 0.90 | 78.51 | 68.49 | 10.02 |
| cg04099158 | 7 | C7orf10 | 3.09577E−13 | 1.14 | 0.73 | 0.57 | 0.90 | 80.60 | 70.59 | 10.01 |
| cg11831419 | 13 | LMO7; LMO7 | 5.31237E−39 | 1.12 | 0.73 | 0.57 | 0.90 | 85.72 | 76.30 | 9.42 |
| cg04521633 | 14 | ATL1 | 1.89026E−12 | 1.13 | 0.73 | 0.57 | 0.90 | 81.81 | 72.40 | 9.41 |
| cg05706517 | 5 | ARSK | 1.65072E−11 | 1.13 | 0.73 | 0.57 | 0.90 | 80.54 | 71.23 | 9.31 |
| cg06838090 | 6 | LOC154449 | 8.89225E−15 | 1.12 | 0.73 | 0.57 | 0.90 | 86.31 | 77.28 | 9.03 |
| cg04879211 | 7 | TNS3 | 5.58524E−11 | 1.12 | 0.73 | 0.57 | 0.90 | 81.49 | 72.62 | 8.87 |
| cg02606496 | 9 | AK1 | 4.92443E−11 | 1.84 | 0.73 | 0.57 | 0.90 | 19.28 | 10.47 | 8.82 |
| cg11719457 | 2 | EXOC6B | 6.22286E−12 | 1.12 | 0.73 | 0.57 | 0.90 | 83.94 | 75.27 | 8.67 |
| cg03948790 | 2 | NEB; NEB; NEB; NEB | 7.9862E−13 | 1.11 | 0.73 | 0.57 | 0.90 | 85.35 | 76.73 | 8.62 |
| cg14850477 | 13 | DACH1; DACH1; DACH1 | 6.39039E−12 | 1.11 | 0.73 | 0.57 | 0.90 | 84.11 | 75.49 | 8.62 |
| cg20311770 | 2 | DTNB; DTNB; DTNB; DTNB; | 5.98512E−12 | 1.10 | 0.73 | 0.57 | 0.90 | 85.86 | 77.70 | 8.16 |
| | | DTNB | | | | | | | | |
| cg10872050 | 7 | NUP205 | 6.48577E−13 | 1.10 | 0.73 | 0.57 | 0.90 | 88.22 | 80.43 | 7.79 |
| cg06602478 | 3 | ACAP2 | 8.26269E−12 | 1.09 | 0.73 | 0.57 | 0.90 | 88.12 | 80.67 | 7.45 |
| cg20333316 | 12 | C1RL-AS1 | 3.51804E−11 | 1.09 | 0.73 | 0.57 | 0.90 | 87.96 | 80.69 | 7.27 |
| cg21361894 | 4 | SLIT2; SLIT2; SLIT2 | 1.32726E−13 | 1.08 | 0.73 | 0.57 | 0.90 | 91.02 | 83.96 | 7.06 |
| cg21950525 | 7 | ZNF655; ZNF655; ZNF655; | 3.42702E−11 | 0.38 | 0.73 | 0.57 | 0.90 | 4.16 | 10.81 | −6.65 |
| | | ZNF655; ZNF655; ZNF655; | | | | | | | | |
| | | ZNF655; ZNF655 | | | | | | | | |
| cg17112155 | 20 | HCK | 3.14419E−12 | 0.35 | 0.73 | 0.57 | 0.90 | 3.71 | 10.55 | −6.84 |
| cg04704193 | 6 | HIST1H3G; HIST1H2BI | 1.66249E−12 | 0.40 | 0.73 | 0.57 | 0.90 | 4.88 | 12.29 | −7.41 |
| cg04987474 | 9 | HDHD3; HDHD3; HDHD3; HDHD3 | 3.79419E−14 | 0.46 | 0.73 | 0.57 | 0.90 | 7.87 | 17.04 | −9.16 |
| cg14898116 | 10 | VIM | 4.17448E−19 | 0.35 | 0.73 | 0.57 | 0.90 | 5.31 | 14.98 | −9.66 |
| cg05886150 | 19 | CA11; SEC1P | 2.98307E−13 | 0.65 | 0.73 | 0.57 | 0.90 | 24.64 | 38.07 | −13.44 |
| cg14885204 | 7 | PTPRZ1; PTPRZ1; PTPRZ1 | 6.42649E−13 | 0.67 | 0.73 | 0.57 | 0.90 | 28.90 | 42.83 | −13.93 |
| cg23777559 | 1 | ALG14; ALG14; ALG14 | 2.46153E−14 | 2.03 | 0.73 | 0.57 | 0.89 | 19.87 | 9.79 | 10.08 |
| cg19888023 | 19 | SCAF1 | 1.18615E−41 | 1.26 | 0.73 | 0.57 | 0.89 | 74.75 | 59.47 | 15.29 |
| cg08831522 | 15 | ATP10A | 2.2768E−40 | 1.20 | 0.73 | 0.57 | 0.89 | 73.95 | 61.40 | 12.55 |
| cg16295676 | 10 | IKZF5 | 4.6019E−13 | 1.21 | 0.73 | 0.57 | 0.89 | 69.33 | 57.34 | 12.00 |
| cg11123847 | 14 | ACTR10 | 2.79183E−14 | 1.19 | 0.73 | 0.57 | 0.89 | 73.57 | 61.68 | 11.88 |
| cg02012712 | 14 | CCNB1IP1; CCNB1IP1; CCNB1IP1; | 1.0879E−11 | 1.21 | 0.73 | 0.57 | 0.89 | 67.36 | 55.78 | 11.58 |
| | | CCNB1IP1 | | | | | | | | |
| cg04437821 | 7 | TAS2R40 | 5.69048E−12 | 1.19 | 0.73 | 0.57 | 0.89 | 70.90 | 59.63 | 11.27 |
| cg01957599 | 4 | FAM13AOS | 1.55093E−13 | 1.17 | 0.73 | 0.57 | 0.89 | 75.94 | 64.80 | 11.14 |
| cg10529381 | 12 | SFSWAP; SFSWAP | 1.18467E−39 | 1.15 | 0.73 | 0.57 | 0.89 | 83.07 | 72.13 | 10.94 |
| cg08359777 | 2 | METTL21A; CREB1; CREB1 | 2.19326E−11 | 1.17 | 0.73 | 0.57 | 0.89 | 72.88 | 62.18 | 10.70 |
| cg03643423 | 17 | MAP2K4 | 2.20385E−12 | 1.16 | 0.73 | 0.57 | 0.89 | 75.80 | 65.14 | 10.66 |
| cg22259344 | 13 | STARD13 | 2.94724E−12 | 1.16 | 0.73 | 0.57 | 0.89 | 77.08 | 66.72 | 10.36 |
| cg22977990 | 4 | MAML3 | 1.14141E−13 | 1.15 | 0.73 | 0.57 | 0.89 | 80.45 | 70.24 | 10.22 |
| cg21403580 | 2 | INPP4A; INPP4A; INPP4A; | 2.49094E−39 | 1.13 | 0.73 | 0.57 | 0.89 | 88.05 | 77.86 | 10.19 |
| | | INPP4A; INPP4A; INPP4A; | | | | | | | | |
| | | INPP4A; INPP4A | | | | | | | | |
| cg00994032 | 14 | SDCCAG1 | 3.03823E−39 | 1.13 | 0.73 | 0.57 | 0.89 | 84.51 | 74.52 | 9.99 |
| cg14605309 | 5 | TARS; TARS; TARS; TARS; TARS; | 2.9279E−14 | 1.14 | 0.73 | 0.57 | 0.89 | 82.56 | 72.64 | 9.92 |
| | | TARS | | | | | | | | |
| cg22300940 | 6 | ABHD16A; ABHD16A; ABHD16A; | 1.99296E−13 | 1.14 | 0.73 | 0.57 | 0.89 | 81.91 | 72.14 | 9.77 |
| | | ABHD16A | | | | | | | | |
| cg15890574 | 15 | DPP8; DPP8; DPP8; DPP8 | 6.39233E−11 | 1.14 | 0.73 | 0.57 | 0.89 | 77.81 | 68.19 | 9.61 |
| cg11068153 | 3 | ARPP21; ARPP21; ARPP21; | 4.20081E−13 | 1.13 | 0.73 | 0.57 | 0.89 | 82.98 | 73.61 | 9.37 |
| | | ARPP21; ARPP21; ARPP21; | | | | | | | | |
| | | ARPP21 | | | | | | | | |
| cg00017887 | 17 | MIR6778; SHMT1; SHMT1; SHMT1 | 1.47207E−11 | 1.13 | 0.73 | 0.57 | 0.89 | 80.67 | 71.37 | 9.30 |
| cg13623384 | 6 | WTAP | 7.74166E−12 | 1.12 | 0.73 | 0.57 | 0.89 | 82.30 | 73.26 | 9.04 |
| cg02591524 | 10 | BAG3 | 4.71033E−11 | 1.11 | 0.73 | 0.57 | 0.89 | 83.71 | 75.34 | 8.37 |
| cg14058932 | 6 | HLA-DRA | 3.53694E−11 | 1.11 | 0.73 | 0.57 | 0.89 | 84.20 | 75.90 | 8.30 |
| cg09554701 | 1 | ACBD6 | 4.90635E−11 | 1.10 | 0.73 | 0.57 | 0.89 | 86.49 | 78.85 | 7.64 |
| cg08903077 | 17 | ZZEF1 | 1.52148E−11 | 1.09 | 0.73 | 0.57 | 0.89 | 88.14 | 80.79 | 7.35 |
| cg26791126 | 1 | DNAJC8 | 1.25433E−14 | 0.41 | 0.73 | 0.57 | 0.89 | 5.90 | 14.44 | −8.54 |
| cg13541429 | 9 | ROR2 | 8.79812E−12 | 0.64 | 0.73 | 0.57 | 0.89 | 21.58 | 33.60 | −12.02 |
| cg12900404 | 2 | DOCK10; DOCK10; DOCK10 | 2.46556E−27 | 0.57 | 0.73 | 0.57 | 0.89 | 26.59 | 46.73 | −20.13 |
| cg15124204 | 6 | GSTA4 | 6.24363E−16 | 0.48 | 0.73 | 0.57 | 0.89 | 9.55 | 20.01 | −10.46 |
| cg23214628 | 7 | YWHAG | 1.00223E−42 | 1.33 | 0.73 | 0.56 | 0.89 | 71.14 | 53.69 | 17.45 |
| cg23221138 | 3 | CLRN1; CLRN1-AS1 | 2.36728E−41 | 1.29 | 0.73 | 0.56 | 0.89 | 64.80 | 50.14 | 14.66 |
| cg21376109 | 6 | PHACTR2 | 7.06804E−41 | 1.23 | 0.73 | 0.56 | 0.89 | 73.71 | 60.06 | 13.65 |
| cg02549424 | 22 | AIFM3; AIFM3 | 1.92261E−13 | 1.41 | 0.73 | 0.56 | 0.89 | 44.88 | 31.76 | 13.12 |
| cg06082709 | 12 | CD63; CD63; CD63; CD63; CD63; | 4.90995E−12 | 1.38 | 0.73 | 0.56 | 0.89 | 45.72 | 33.23 | 12.49 |
| | | CD63; CD63; CD63; CD63 | | | | | | | | |
| cg07143733 | 21 | MIR155HG | 1.60034E−11 | 1.29 | 0.73 | 0.56 | 0.89 | 54.65 | 42.33 | 12.32 |
| cg14214460 | 2 | GTDC1; GTDC1; GTDC1 | 5.07064E−15 | 1.18 | 0.73 | 0.56 | 0.89 | 76.22 | 64.51 | 11.71 |
| cg03537802 | 11 | KIAA1377 | 8.71803E−15 | 1.18 | 0.73 | 0.56 | 0.89 | 75.78 | 64.11 | 11.66 |
| cg17012555 | 2 | PSMD1 | 6.16147E−40 | 1.17 | 0.73 | 0.56 | 0.89 | 80.70 | 69.12 | 11.58 |
| cg14281039 | 19 | TNNI3 | 9.26099E−12 | 1.20 | 0.73 | 0.56 | 0.89 | 69.05 | 57.64 | 11.41 |
| cg01710742 | 5 | LINC00992 | 3.69701E−11 | 1.20 | 0.73 | 0.56 | 0.89 | 68.14 | 56.92 | 11.22 |
| cg17729365 | 19 | SLC44A2; SLC44A2 | 5.67846E−11 | 1.19 | 0.73 | 0.56 | 0.89 | 68.74 | 57.69 | 11.05 |
| cg22626281 | 14 | SERPINA10; SERPINA10 | 2.22748E−14 | 1.16 | 0.73 | 0.56 | 0.89 | 78.25 | 67.25 | 11.00 |
| cg07709195 | 11 | FADS1 | 2.5081E−14 | 1.16 | 0.73 | 0.56 | 0.89 | 78.47 | 67.53 | 10.94 |
| cg18468844 | 1 | PTAFR; PTAFR; PTAFR; PTAFR | 2.56924E−11 | 1.18 | 0.73 | 0.56 | 0.89 | 71.47 | 60.60 | 10.87 |
| cg20099449 | 18 | DSG1 | 1.27121E−39 | 1.15 | 0.73 | 0.56 | 0.89 | 81.24 | 70.38 | 10.87 |
| cg01427849 | 8 | TOX | 1.27873E−39 | 1.15 | 0.73 | 0.56 | 0.89 | 85.71 | 74.85 | 10.86 |
| cg14618923 | 6 | FOXO3; FOXO3 | 5.40938E−12 | 1.17 | 0.73 | 0.56 | 0.89 | 74.37 | 63.62 | 10.74 |
| cg07588148 | 5 | DDX4; DDX4; DDX4; DDX4 | 4.72497E−12 | 1.17 | 0.73 | 0.56 | 0.89 | 74.66 | 63.94 | 10.72 |
| cg21108859 | 12 | PSMD9 | 2.44514E−15 | 1.15 | 0.73 | 0.56 | 0.89 | 81.45 | 70.81 | 10.63 |
| cg15625785 | 5 | SAR1B; SAR1B | 1.7125E−39 | 1.14 | 0.73 | 0.56 | 0.89 | 84.61 | 74.04 | 10.57 |
| cg15612777 | 20 | TMC2 | 1.11501E−12 | 1.15 | 0.73 | 0.56 | 0.89 | 79.26 | 69.17 | 10.09 |
| cg25352342 | 4 | FAT1 | 3.39273E−39 | 1.13 | 0.73 | 0.56 | 0.89 | 86.24 | 76.36 | 9.88 |
| cg03968618 | 4 | SORBS2 | 6.41461E−12 | 1.14 | 0.73 | 0.56 | 0.89 | 79.36 | 69.61 | 9.75 |
| cg00912746 | 1 | HPCA | 9.02857E−12 | 1.14 | 0.73 | 0.56 | 0.89 | 79.18 | 69.46 | 9.72 |
| cg12934016 | 15 | UBE3A; UBE3A | 3.29951E−11 | 1.14 | 0.73 | 0.56 | 0.89 | 78.34 | 68.69 | 9.64 |
| cg27445709 | 7 | C7orf65 | 8.18393E−12 | 1.14 | 0.73 | 0.56 | 0.89 | 79.84 | 70.24 | 9.60 |
| cg04945860 | 8 | MIR1207; PVT1 | 6.54452E−11 | 1.14 | 0.73 | 0.56 | 0.89 | 78.71 | 69.27 | 9.43 |
| cg23682469 | 4 | CSN1S1; CSN1S1 | 3.63696E−11 | 1.13 | 0.73 | 0.56 | 0.89 | 79.72 | 70.38 | 9.34 |
| cg07392941 | 2 | INO80D | 2.05505E−14 | 1.12 | 0.73 | 0.56 | 0.89 | 85.11 | 75.85 | 9.26 |
| cg00051885 | 3 | IQCF6 | 1.17118E−12 | 1.12 | 0.73 | 0.56 | 0.89 | 83.69 | 74.68 | 9.01 |
| cg18123143 | 8 | TRPS1; TRPS1; TRPS1 | 3.59079E−13 | 1.11 | 0.73 | 0.56 | 0.89 | 85.39 | 76.66 | 8.74 |
| cg00470636 | 8 | DPYSL2 | 2.21407E−11 | 1.11 | 0.73 | 0.56 | 0.89 | 83.46 | 74.90 | 8.57 |
| cg16803678 | 3 | RYK; RYK | 3.18623E−11 | 1.11 | 0.73 | 0.56 | 0.89 | 83.21 | 74.64 | 8.57 |
| cg11457582 | 7 | MAD1L1; MAD1L1; MAD1L1 | 5.84561E−15 | 1.10 | 0.73 | 0.56 | 0.89 | 88.63 | 80.37 | 8.26 |
| cg09952002 | 17 | HOXB3 | 3.14753E−11 | 1.11 | 0.73 | 0.56 | 0.89 | 85.02 | 76.92 | 8.11 |
| cg09472026 | 8 | MCM4; MCM4 | 9.15449E−13 | 1.10 | 0.73 | 0.56 | 0.89 | 87.97 | 80.14 | 7.82 |
| cg17237927 | 16 | BANP; BANP; BANP; BANP; | 4.23768E−11 | 1.08 | 0.73 | 0.56 | 0.89 | 89.09 | 82.18 | 6.91 |
| | | BANP; BANP | | | | | | | | |
| cg19988204 | 8 | MAPK15 | 2.29888E−15 | 0.42 | 0.73 | 0.56 | 0.89 | 6.74 | 15.88 | −9.14 |
| cg20390711 | 6 | C6orf174 | 3.29468E−11 | 0.87 | 0.73 | 0.56 | 0.89 | 71.29 | 82.09 | −10.80 |
| cg03559406 | 8 | CHD7 | 2.22499E−12 | 0.68 | 0.73 | 0.56 | 0.89 | 28.27 | 41.82 | −13.54 |
| cg10191855 | 2 | IKZF2; IKZF2; IKZF2; IKZF2 | 3.15989E−22 | 0.63 | 0.73 | 0.56 | 0.89 | 31.72 | 50.57 | −18.85 |
| cg02336817 | 16 | FTO | 2.74187E−41 | 1.23 | 0.72 | 0.56 | 0.89 | 77.21 | 62.68 | 14.53 |
| cg24024511 | 7 | C7orf71 | 3.7495E−40 | 1.18 | 0.72 | 0.56 | 0.89 | 78.45 | 66.38 | 12.07 |
| cg21113768 | 6 | PLAGL1; PLAGL1 | 3.756E−40 | 1.18 | 0.72 | 0.56 | 0.89 | 77.28 | 65.21 | 12.06 |
| cg27078890 | 11 | ETS1 | 2.0891E−13 | 1.20 | 0.72 | 0.56 | 0.89 | 71.05 | 59.14 | 11.91 |
| cg15535628 | 3 | TP63; TP63; TP63; TP63; TP63; | 1.03422E−14 | 1.18 | 0.72 | 0.56 | 0.89 | 75.52 | 63.81 | 11.70 |
| | | TP63 | | | | | | | | |
| cg17444770 | 12 | CLEC1A; CLEC1A; CLEC1A; | 1.71264E−15 | 1.18 | 0.72 | 0.56 | 0.89 | 77.61 | 66.04 | 11.57 |
| | | CLEC1A; CLEC1A | | | | | | | | |
| cg19740353 | 6 | GCLC | 1.17811E−13 | 1.18 | 0.72 | 0.56 | 0.89 | 74.45 | 62.98 | 11.46 |
| cg13451819 | 7 | IMMP2L; IMMP2L | 4.37136E−13 | 1.17 | 0.72 | 0.56 | 0.89 | 75.48 | 64.45 | 11.03 |
| cg10890016 | 7 | UNC84A; UNC84A | 1.302E−39 | 1.15 | 0.72 | 0.56 | 0.89 | 84.54 | 73.70 | 10.84 |
| cg10338001 | 5 | EPB41L4A | 2.74463E−11 | 1.17 | 0.72 | 0.56 | 0.89 | 73.01 | 62.38 | 10.63 |
| cg15569001 | 1 | TM2D1 | 4.24832E−14 | 1.15 | 0.72 | 0.56 | 0.89 | 79.98 | 69.48 | 10.50 |
| cg18354203 | 11 | BDNF; BDNF; BDNF; BDNF; | 5.43686E−15 | 1.15 | 0.72 | 0.56 | 0.89 | 81.74 | 71.35 | 10.39 |
| | | BDNF; BDNF; BDNF; BDNF; | | | | | | | | |
| | | BDNF; BDNF; BDNF; BDNF; | | | | | | | | |
| | | BDNF; BDNF; BDNF; | | | | | | | | |
| | | BDNFOS; BDNF | | | | | | | | |
| cg06398242 | 2 | PRKCE | 2.69496E−15 | 1.14 | 0.72 | 0.56 | 0.89 | 82.20 | 71.82 | 10.37 |
| cg26049080 | 12 | LOC374443; LOC374443; | 1.17724E−11 | 1.16 | 0.72 | 0.56 | 0.89 | 75.57 | 65.20 | 10.37 |
| | | LOC374443 | | | | | | | | |
| cg11943389 | 3 | DNAJC13 | 7.1088E−12 | 1.15 | 0.72 | 0.56 | 0.89 | 76.74 | 66.49 | 10.26 |
| cg11593346 | 14 | TTLL5 | 1.93845E−11 | 1.15 | 0.72 | 0.56 | 0.89 | 76.49 | 66.39 | 10.11 |
| cg05796845 | 3 | MBNL1; MBNL1; MBNL1; | 4.05527E−12 | 1.15 | 0.72 | 0.56 | 0.89 | 78.27 | 68.21 | 10.06 |
| | | MBNL1; MBNL1; MBNL1; | | | | | | | | |
| | | MBNL1; TMEM14E | | | | | | | | |
| cg13290564 | 6 | HCRTR2 | 3.49898E−13 | 1.14 | 0.72 | 0.56 | 0.89 | 81.27 | 71.45 | 9.83 |
| cg27545565 | 4 | PALLD; PALLD; PALLD | 4.77513E−12 | 1.14 | 0.72 | 0.56 | 0.89 | 79.79 | 70.08 | 9.71 |
| cg07547798 | 8 | NSMCE2 | 1.23546E−11 | 1.14 | 0.72 | 0.56 | 0.89 | 79.47 | 69.87 | 9.60 |
| cg07899244 | 17 | UBE2G1 | 4.92787E−11 | 1.14 | 0.72 | 0.56 | 0.89 | 78.35 | 68.79 | 9.56 |
| cg14498592 | 20 | KCNQ2; KCNQ2; KCNQ2; KCNQ2; | 1.52817E−13 | 1.13 | 0.72 | 0.56 | 0.89 | 83.48 | 74.07 | 9.40 |
| | | KCNQ2 | | | | | | | | |
| cg11105743 | 18 | PTPN2; PTPN2; PTPN2; PTPN2 | 5.18621E−12 | 1.13 | 0.72 | 0.56 | 0.89 | 81.43 | 72.11 | 9.32 |
| cg08004814 | 1 | LOC101928436 | 9.25015E−12 | 1.13 | 0.72 | 0.56 | 0.89 | 81.17 | 71.89 | 9.28 |
| cg15998773 | 1 | RSC1A1 | 7.69788E−39 | 1.12 | 0.72 | 0.56 | 0.89 | 87.42 | 78.38 | 9.04 |
| cg11213278 | 4 | CNOT6L; CNOT6L | 5.19295E−12 | 1.12 | 0.72 | 0.56 | 0.89 | 83.86 | 75.14 | 8.72 |
| cg08976790 | 19 | MUM1; MUM1 | 1.24789E−11 | 1.11 | 0.72 | 0.56 | 0.89 | 83.65 | 75.03 | 8.62 |
| cg26652131 | 19 | ZNF665 | 4.79575E−12 | 1.11 | 0.72 | 0.56 | 0.89 | 85.22 | 76.85 | 8.37 |
| cg07144666 | 7 | KDELR2; KDELR2 | 2.55722E−12 | 1.10 | 0.72 | 0.56 | 0.89 | 86.50 | 78.39 | 8.11 |
| cg12846232 | 10 | MGEA5; MGEA5 | 5.04436E−11 | 1.10 | 0.72 | 0.56 | 0.89 | 85.97 | 78.20 | 7.77 |
| cg14700609 | 13 | PCOTH; MIPEP; PCOTH | 4.35306E−12 | 0.44 | 0.72 | 0.56 | 0.89 | 6.17 | 13.92 | −7.75 |
| cg15978276 | 1 | PIAS3 | 7.3938E−13 | 0.43 | 0.72 | 0.56 | 0.89 | 6.03 | 14.03 | −8.00 |
| cg17187559 | 10 | ZNF248 | 1.86522E−14 | 0.40 | 0.72 | 0.56 | 0.89 | 5.45 | 13.75 | −8.30 |
| cg25932239 | 12 | SNRNP35; SNRNP35; SNRNP35 | 4.73834E−18 | 0.29 | 0.72 | 0.56 | 0.89 | 3.53 | 12.01 | −8.48 |
| cg13848802 | 4 | SMARCA5 | 2.63819E−12 | 0.55 | 0.72 | 0.56 | 0.89 | 12.32 | 22.26 | −9.94 |
| cg06825627 | 8 | SLC7A2 | 7.07219E−18 | 0.53 | 0.72 | 0.56 | 0.89 | 14.76 | 27.84 | −13.08 |
| cg08458921 | 6 | RANBP9 | 1.09085E−13 | 1.45 | 0.72 | 0.56 | 0.89 | 42.09 | 29.02 | 13.07 |
| cg03301354 | 12 | ERC1; ERC1; ERC1; ERC1; ERC1; | 2.76285E−13 | 1.26 | 0.72 | 0.56 | 0.89 | 62.22 | 49.38 | 12.84 |
| | | ERC1 | | | | | | | | |
| cg00139234 | 6 | QKI; QKI; QKI; QKI; QKI | 1.94089E−12 | 1.34 | 0.72 | 0.56 | 0.89 | 50.34 | 37.54 | 12.81 |
| cg07626798 | 9 | PHF2 | 2.54604E−40 | 1.18 | 0.72 | 0.56 | 0.89 | 80.97 | 68.53 | 12.44 |
| cg21143551 | 6 | EPM2A; EPM2A; LOC100507557 | 8.09133E−12 | 1.37 | 0.72 | 0.56 | 0.89 | 46.18 | 33.78 | 12.40 |
| cg27280022 | 12 | MIR135A2 | 4.98054E−12 | 1.21 | 0.72 | 0.56 | 0.89 | 67.39 | 55.64 | 11.75 |
| cg04147428 | 7 | MET; MET | 4.10312E−14 | 1.17 | 0.72 | 0.56 | 0.89 | 76.50 | 65.23 | 11.27 |
| cg07262012 | 7 | RBM33 | 9.74205E−40 | 1.16 | 0.72 | 0.56 | 0.89 | 82.90 | 71.77 | 11.13 |
| cg17940201 | 11 | USP47; USP47 | 1.04955E−39 | 1.15 | 0.72 | 0.56 | 0.89 | 82.51 | 71.46 | 11.06 |
| cg26932252 | 3 | ARHGEF3; ARHGEF3; ARHGEF3; | 9.27075E−12 | 1.18 | 0.72 | 0.56 | 0.89 | 71.80 | 60.76 | 11.04 |
| | | ARHGEF3 | | | | | | | | |
| cg01632220 | 2 | SLC30A6; SLC30A6; SLC30A6; | 8.83691E−15 | 1.16 | 0.72 | 0.56 | 0.89 | 78.97 | 67.97 | 11.00 |
| | | SLC30A6 | | | | | | | | |
| cg11445109 | 10 | CYP2E1 | 1.65057E−39 | 2.86 | 0.72 | 0.56 | 0.89 | 16.32 | 5.71 | 10.61 |
| cg23677229 | 14 | GPHN; GPHN | 5.52726E−11 | 1.17 | 0.72 | 0.56 | 0.89 | 72.51 | 61.95 | 10.55 |
| cg03439613 | 1 | MEAF6; MEAF6; MEAF6; MEAF6; | 4.29191E−11 | 1.16 | 0.72 | 0.56 | 0.89 | 73.91 | 63.52 | 10.39 |
| | | MEAF6; MEAF6 | | | | | | | | |
| cg07571282 | 16 | BCMO1 | 8.72431E−12 | 1.15 | 0.72 | 0.56 | 0.89 | 77.45 | 67.37 | 10.08 |
| cg19094597 | 12 | EMP1 | 3.42147E−39 | 1.13 | 0.72 | 0.56 | 0.89 | 84.72 | 74.85 | 9.87 |
| cg13558754 | 19 | HSPB6; C19orf55 | 6.32154E−11 | 1.65 | 0.72 | 0.56 | 0.89 | 24.71 | 14.93 | 9.77 |
| cg17437621 | 6 | SFRS18; SFRS18 | 3.72193E−13 | 1.13 | 0.72 | 0.56 | 0.89 | 81.89 | 72.23 | 9.67 |
| cg15133748 | 2 | ALLC | 4.74474E−39 | 1.12 | 0.72 | 0.56 | 0.89 | 89.79 | 80.25 | 9.54 |
| cg20414506 | 9 | CAMSAP1 | 1.20915E−11 | 1.13 | 0.72 | 0.56 | 0.89 | 80.50 | 71.12 | 9.38 |
| cg01059379 | 15 | TMOD3 | 8.00206E−39 | 1.11 | 0.72 | 0.56 | 0.89 | 87.68 | 78.69 | 9.00 |
| cg00853189 | 7 | UBE3C | 4.09123E−11 | 1.12 | 0.72 | 0.56 | 0.89 | 81.49 | 72.57 | 8.93 |
| cg08840929 | 6 | MOXD1 | 3.11781E−11 | 1.12 | 0.72 | 0.56 | 0.89 | 81.79 | 72.88 | 8.91 |
| cg10331412 | 19 | ZNF574 | 1.04405E−38 | 1.11 | 0.72 | 0.56 | 0.89 | 87.90 | 79.18 | 8.72 |
| cg08693937 | 2 | MPP4 | 1.526E−11 | 1.12 | 0.72 | 0.56 | 0.89 | 83.42 | 74.77 | 8.65 |
| cg01782059 | 1 | ERI3 | 9.25328E−12 | 1.07 | 0.72 | 0.56 | 0.89 | 91.73 | 85.47 | 6.26 |
| cg00480356 | 15 | SERINC4; C15orf63 | 1.1909E−11 | 0.41 | 0.72 | 0.56 | 0.89 | 4.96 | 12.08 | −7.12 |
| cg19489111 | 4 | RG9MTD2; RG9MTD2; MTTP; | 1.47968E−17 | 0.32 | 0.72 | 0.56 | 0.89 | 4.15 | 12.80 | −8.65 |
| | | RG9MTD2 | | | | | | | | |
| cg10596483 | 8 | JRK; JRK | 4.75543E−22 | 0.56 | 0.72 | 0.56 | 0.89 | 21.80 | 38.69 | −16.89 |
| cg20112147 | 2 | FMNL2 | 2.0321E−41 | 1.26 | 0.72 | 0.55 | 0.88 | 72.22 | 57.42 | 14.80 |
| cg23137088 | 5 | H2AFY | 2.92784E−15 | 1.24 | 0.72 | 0.55 | 0.88 | 67.86 | 54.69 | 13.16 |
| cg20171775 | 2 | ZEB2; ZEB2 | 1.46558E−12 | 1.25 | 0.72 | 0.55 | 0.88 | 61.82 | 49.30 | 12.52 |
| cg08146441 | 4 | GRID2; GRID2 | 7.78286E−12 | 1.32 | 0.72 | 0.55 | 0.88 | 51.29 | 38.77 | 12.51 |
| cg09799529 | 5 | CREBRF | 2.72451E−40 | 1.18 | 0.72 | 0.55 | 0.88 | 83.01 | 70.63 | 12.37 |
| cg04350236 | 8 | TAF2; TAF2 | 3.44516E−40 | 1.19 | 0.72 | 0.55 | 0.88 | 77.15 | 65.01 | 12.15 |
| cg21196439 | 6 | AIG1; AIG1; AIG1; AIG1 | 8.17933E−40 | 1.16 | 0.72 | 0.55 | 0.88 | 80.11 | 68.80 | 11.30 |
| cg02535680 | 19 | LINC00904 | 4.24521E−12 | 1.19 | 0.72 | 0.55 | 0.88 | 71.61 | 60.38 | 11.23 |
| cg03457345 | 10 | PCGF6; PCGF6 | 9.01736E−40 | 1.16 | 0.72 | 0.55 | 0.88 | 80.82 | 69.62 | 11.21 |
| cg16134098 | 14 | G2E3; G2E3 | 2.10233E−13 | 1.17 | 0.72 | 0.55 | 0.88 | 75.57 | 64.42 | 11.15 |
| cg26134453 | 12 | GUCY2C | 1.26615E−39 | 1.15 | 0.72 | 0.55 | 0.88 | 81.44 | 70.57 | 10.87 |
| cg14778914 | 17 | ARSG | 6.58426E−13 | 1.16 | 0.72 | 0.55 | 0.88 | 76.37 | 65.59 | 10.78 |
| cg03327221 | 10 | USP6NL; USP6NL | 3.15087E−14 | 1.16 | 0.72 | 0.55 | 0.88 | 79.04 | 68.28 | 10.77 |
| cg16201893 | 9 | AKAP2; PALM2-AKAP2; | 9.65919E−15 | 1.16 | 0.72 | 0.55 | 0.88 | 79.98 | 69.22 | 10.76 |
| | | PALM2-AKAP2; AKAP2; AKAP2 | | | | | | | | |
| cg03974292 | 6 | MANEA | 1.64607E−39 | 1.15 | 0.72 | 0.55 | 0.88 | 83.08 | 72.47 | 10.61 |
| cg16037569 | 1 | PIK3CD | 2.13449E−39 | 1.14 | 0.72 | 0.55 | 0.88 | 86.69 | 76.35 | 10.35 |
| cg05859533 | 16 | CCDC135 | 2.42531E−39 | 1.14 | 0.72 | 0.55 | 0.88 | 83.78 | 73.56 | 10.22 |
| cg01012394 | 1 | EYA3 | 1.61277E−14 | 1.14 | 0.72 | 0.55 | 0.88 | 82.78 | 72.83 | 9.95 |
| cg20758929 | 4 | PCGF3 | 3.99635E−14 | 1.14 | 0.72 | 0.55 | 0.88 | 82.58 | 72.73 | 9.85 |
| cg07355189 | 10 | ALOX5 | 1.22273E−13 | 1.14 | 0.72 | 0.55 | 0.88 | 81.93 | 72.08 | 9.85 |
| cg00695244 | 7 | IQUB; IQUB; IQUB; IQUB | 2.41544E−12 | 1.14 | 0.72 | 0.55 | 0.88 | 80.13 | 70.37 | 9.76 |
| cg05873349 | 10 | ZNF239; ZNF239; ZNF239 | 1.08021E−12 | 1.13 | 0.72 | 0.55 | 0.88 | 81.71 | 72.18 | 9.53 |
| cg17947789 | 12 | PTPN11; PTPN11 | 1.16964E−11 | 1.14 | 0.72 | 0.55 | 0.88 | 79.87 | 70.35 | 9.52 |
| cg00401471 | 19 | SPINT2; SPINT2 | 4.47019E−11 | 1.14 | 0.72 | 0.55 | 0.88 | 79.16 | 69.75 | 9.42 |
| cg00004306 | 6 | TAAR2; TAAR2 | 6.89715E−12 | 1.13 | 0.72 | 0.55 | 0.88 | 81.31 | 72.01 | 9.30 |
| cg03900431 | 1 | RABGAP1L | 6.2908E−12 | 1.12 | 0.72 | 0.55 | 0.88 | 82.85 | 73.90 | 8.94 |
| cg24130271 | 11 | KIAA1549L | 2.40969E−12 | 1.12 | 0.72 | 0.55 | 0.88 | 83.60 | 74.68 | 8.92 |
| cg07406797 | 2 | NOL10; NOL10; NOL10; NOL10 | 9.1678E−13 | 1.11 | 0.72 | 0.55 | 0.88 | 85.34 | 76.74 | 8.60 |
| cg01923516 | 7 | SDK1 | 5.44585E−11 | 1.11 | 0.72 | 0.55 | 0.88 | 84.58 | 76.46 | 8.13 |
| cg27004481 | 4 | PDLIM5; PDLIM5; PDLIM5; | 2.06885E−11 | 1.10 | 0.72 | 0.55 | 0.88 | 85.74 | 77.76 | 7.99 |
| | | PDLIM5; PDLIM5; PDLIM5; | | | | | | | | |
| | | PDLIM5; PDLIM5; PDLIM5; | | | | | | | | |
| | | PDLIM5 | | | | | | | | |
| cg22563998 | 1 | ARHGAP30; ARHGAP30; | 1.41027E−11 | 1.10 | 0.72 | 0.55 | 0.88 | 86.68 | 78.90 | 7.79 |
| | | ARHGAP30; ARHGAP30; PVRL4 | | | | | | | | |
| cg10955092 | 2 | FHL2; FHL2; FHL2; FHL2 | 4.52552E−13 | 1.10 | 0.72 | 0.55 | 0.88 | 88.53 | 80.79 | 7.74 |
| cg27394892 | 3 | CADPS; CADPS; CADPS | 1.48611E−11 | 1.09 | 0.72 | 0.55 | 0.88 | 88.17 | 80.82 | 7.34 |
| cg23649190 | 22 | ZBED4 | 1.93936E−14 | 1.08 | 0.72 | 0.55 | 0.88 | 92.41 | 85.63 | 6.78 |
| cg25061131 | 11 | RELT; RELT | 1.4113E−13 | 0.41 | 0.72 | 0.55 | 0.88 | 5.71 | 13.83 | −8.12 |
| cg16556111 | 5 | NR2F1 | 2.22251E−14 | 0.46 | 0.72 | 0.55 | 0.88 | 7.80 | 17.02 | −9.22 |
| cg26215849 | 13 | MTRF1; MTRF1 | 2.13529E−19 | 0.52 | 0.72 | 0.55 | 0.88 | 15.01 | 28.78 | −13.77 |
| cg18652204 | 14 | BAZ1A; BAZ1A | 1.28441E−12 | 0.47 | 0.72 | 0.55 | 0.88 | 7.45 | 15.89 | −8.43 |
| cg17448954 | 21 | NCAM2 | 4.67306E−42 | 1.41 | 0.71 | 0.55 | 0.88 | 55.80 | 39.68 | 16.12 |
| cg24441954 | 6 | ZSCAN23 | 7.71791E−42 | 1.30 | 0.71 | 0.55 | 0.88 | 68.35 | 52.68 | 15.67 |
| cg21219570 | 12 | PLEKHA5; PLEKHA5; PLEKHA5; | 8.83962E−13 | 1.38 | 0.71 | 0.55 | 0.88 | 46.88 | 33.98 | 12.90 |
| | | PLEKHA5 | | | | | | | | |
| cg06070446 | 2 | GALNT3 | 2.53133E−15 | 1.21 | 0.71 | 0.55 | 0.88 | 72.84 | 60.42 | 12.43 |
| cg14904829 | 3 | LINC00882 | 9.48029E−14 | 1.22 | 0.71 | 0.55 | 0.88 | 68.69 | 56.30 | 12.39 |
| cg23858360 | 10 | TSPAN14; TSPAN14 | 3.38988E−12 | 1.42 | 0.71 | 0.55 | 0.88 | 42.09 | 29.71 | 12.38 |
| cg05068534 | 18 | SERPINB4; SERPINB4 | 2.69743E−40 | 1.18 | 0.71 | 0.55 | 0.88 | 80.53 | 68.15 | 12.38 |
| cg04462334 | 8 | UQCRB | 4.40916E−40 | 1.18 | 0.71 | 0.55 | 0.88 | 77.87 | 65.97 | 11.91 |
| cg20178011 | 2 | EXOC6B | 6.58158E−15 | 1.19 | 0.71 | 0.55 | 0.88 | 75.32 | 63.51 | 11.81 |
| cg27116069 | 14 | SYNE2; SYNE2; SYNE2; SYNE2; | 3.04636E−13 | 1.19 | 0.71 | 0.55 | 0.88 | 72.58 | 60.98 | 11.60 |
| | | MIR548AZ | | | | | | | | |
| cg25074809 | 2 | MYCN | 1.70376E−15 | 1.18 | 0.71 | 0.55 | 0.88 | 77.48 | 65.89 | 11.58 |
| cg15266307 | 15 | AGPHD1; AGPHD1 | 7.08754E−40 | 1.16 | 0.71 | 0.55 | 0.88 | 82.20 | 70.76 | 11.44 |
| cg18348731 | 18 | TUBB6 | 3.28012E−14 | 1.18 | 0.71 | 0.55 | 0.88 | 75.87 | 64.44 | 11.43 |
| cg07344019 | 15 | MIR548H4; NOX5 | 7.76848E−40 | 1.17 | 0.71 | 0.55 | 0.88 | 80.03 | 68.67 | 11.35 |
| cg25235770 | 12 | PPM1H | 5.76897E−13 | 1.17 | 0.71 | 0.55 | 0.88 | 74.96 | 63.89 | 11.07 |
| cg21750589 | 6 | ZNF323; ZKSCAN3; ZNF323 | 7.15649E−12 | 1.17 | 0.71 | 0.55 | 0.88 | 74.15 | 63.43 | 10.72 |
| cg25426630 | 12 | DDX11; DDX11; DDX11 | 6.73892E−14 | 1.15 | 0.71 | 0.55 | 0.88 | 79.25 | 68.66 | 10.59 |
| cg16732415 | 4 | C4orf11; C4orf11 | 5.23147E−12 | 1.16 | 0.71 | 0.55 | 0.88 | 76.26 | 65.85 | 10.41 |
| cg24947985 | 17 | ZNF624 | 5.28085E−12 | 1.16 | 0.71 | 0.55 | 0.88 | 76.59 | 66.24 | 10.35 |
| cg13738964 | 12 | ZNF26; ZNF26; ZNF26 | 2.36521E−39 | 1.14 | 0.71 | 0.55 | 0.88 | 84.11 | 73.86 | 10.24 |
| cg06204066 | 6 | DNAH8 | 1.03678E−13 | 1.14 | 0.71 | 0.55 | 0.88 | 81.41 | 71.41 | 10.00 |
| cg15986307 | 7 | GNA12; GNA12; GNA12 | 3.61157E−11 | 1.15 | 0.71 | 0.55 | 0.88 | 77.30 | 67.47 | 9.83 |
| cg04439557 | 1 | NADK | 4.48066E−13 | 1.14 | 0.71 | 0.55 | 0.88 | 81.11 | 71.28 | 9.83 |
| cg09841898 | 14 | MAP4K5; MAP4K5 | 8.66417E−12 | 1.14 | 0.71 | 0.55 | 0.88 | 79.93 | 70.37 | 9.57 |
| cg03878567 | 6 | COL9A1; COL9A1 | 1.31711E−11 | 1.13 | 0.71 | 0.55 | 0.88 | 80.30 | 70.90 | 9.41 |
| cg21263170 | 13 | SLC7A1 | 2.44174E−12 | 1.13 | 0.71 | 0.55 | 0.88 | 81.88 | 72.54 | 9.35 |
| cg02727723 | 17 | C17orf63; C17orf63 | 1.65115E−11 | 1.13 | 0.71 | 0.55 | 0.88 | 80.61 | 71.31 | 9.30 |
| cg27122065 | 6 | GTF2H4 | 8.10035E−15 | 1.12 | 0.71 | 0.55 | 0.88 | 85.58 | 76.29 | 9.29 |
| cg10176510 | 6 | NDUFAF4 | 4.41166E−14 | 1.11 | 0.71 | 0.55 | 0.88 | 86.50 | 77.78 | 8.72 |
| cg24939330 | 14 | MIR3171; LINC00645 | 1.40616E−12 | 1.11 | 0.71 | 0.55 | 0.88 | 85.06 | 76.44 | 8.61 |
| cg06609843 | 3 | C3orf50 | 8.78467E−12 | 1.11 | 0.71 | 0.55 | 0.88 | 84.07 | 75.50 | 8.58 |
| cg00475194 | 2 | PMS1; PMS1; PMS1 | 2.38341E−11 | 1.11 | 0.71 | 0.55 | 0.88 | 83.99 | 75.57 | 8.42 |
| cg16481280 | 6 | PPT2; PRRT1; PPT2 | 1.43356E−38 | 1.10 | 0.71 | 0.55 | 0.88 | 88.88 | 80.49 | 8.39 |
| cg19330506 | 11 | MSANTD2; MSANTD2; MSANTD2 | 4.12829E−11 | 1.10 | 0.71 | 0.55 | 0.88 | 85.28 | 77.29 | 7.99 |
| cg15609478 | 17 | METTL16 | 3.69308E−11 | 1.10 | 0.71 | 0.55 | 0.88 | 85.98 | 78.16 | 7.82 |
| cg06199363 | 3 | ZDHHC3; ZDHHC3 | 5.62807E−12 | 1.10 | 0.71 | 0.55 | 0.88 | 87.54 | 79.87 | 7.68 |
| cg16874710 | 13 | SLC7A1 | 2.50645E−12 | 1.09 | 0.71 | 0.55 | 0.88 | 88.45 | 80.92 | 7.52 |
| cg15048972 | 7 | MAD1L1; MAD1L1; MAD1L1 | 2.96418E−11 | 1.09 | 0.71 | 0.55 | 0.88 | 88.49 | 81.36 | 7.14 |
| cg27534415 | 2 | NEB; NEB; NEB; NEB; NEB; NEB; | 4.46262E−11 | 1.08 | 0.71 | 0.55 | 0.88 | 88.94 | 82.00 | 6.94 |
| | | NEB; NEB | | | | | | | | |
| cg08111857 | 17 | CNTNAP1 | 1.22308E−13 | 1.08 | 0.71 | 0.55 | 0.88 | 92.03 | 85.33 | 6.70 |
| cg20337934 | 2 | SNRNP27 | 8.24025E−14 | 0.38 | 0.71 | 0.55 | 0.88 | 4.87 | 12.72 | −7.85 |
| cg05570178 | 1 | SCP2; SCP2; SCP2; SCP2; SCP2; | 3.1396E−15 | 0.48 | 0.71 | 0.55 | 0.88 | 9.09 | 19.11 | −10.02 |
| | | SCP2; SCP2 | | | | | | | | |
| cg07128799 | 10 | TCF7L2; TCF7L2; TCF7L2; | 1.91736E−16 | 0.47 | 0.71 | 0.55 | 0.88 | 9.23 | 19.75 | −10.52 |
| | | TCF7L2; TCF7L2; TCF7L2 | | | | | | | | |
| cg11646986 | 2 | PID1; PID1 | 4.44934E−12 | 0.70 | 0.71 | 0.55 | 0.88 | 32.74 | 46.62 | −13.88 |
| cg06474219 | 6 | POLR1C | 1.92499E−41 | 1.26 | 0.71 | 0.54 | 0.88 | 72.56 | 57.71 | 14.85 |
| cg07247255 | 2 | CSRNP3; CSRNP3 | 6.61644E−15 | 1.26 | 0.71 | 0.54 | 0.88 | 64.79 | 51.46 | 13.33 |
| cg12494208 | 17 | CLTC | 1.78003E−40 | 1.21 | 0.71 | 0.54 | 0.88 | 74.53 | 61.74 | 12.78 |
| cg11644515 | 21 | KCNE1; KCNE1; KCNE1; KCNE1; | 2.04201E−40 | 1.20 | 0.71 | 0.54 | 0.88 | 74.66 | 62.01 | 12.65 |
| | | KCNE1 | | | | | | | | |
| cg25009697 | 22 | SGSM1; SGSM1; SGSM1; SGSM1 | 1.76146E−13 | 1.22 | 0.71 | 0.54 | 0.88 | 68.45 | 56.15 | 12.30 |
| cg03486265 | 7 | PHTF2; PHTF2 | 3.45572E−40 | 1.17 | 0.71 | 0.54 | 0.88 | 82.32 | 70.18 | 12.14 |
| cg07679697 | 1 | COG2; COG2 | 9.05387E−13 | 1.19 | 0.71 | 0.54 | 0.88 | 71.19 | 59.58 | 11.60 |
| cg12335815 | 16 | LCMT1; LCMT1 | 8.47329E−40 | 1.15 | 0.71 | 0.54 | 0.88 | 84.29 | 73.02 | 11.27 |
| cg10102721 | 1 | AMY2B | 3.4913E−12 | 1.18 | 0.71 | 0.54 | 0.88 | 72.48 | 61.35 | 11.14 |
| cg10667857 | 16 | MIR548AE2; LONP2; LONP2; | 1.34021E−12 | 1.18 | 0.71 | 0.54 | 0.88 | 73.75 | 62.63 | 11.12 |
| | | MIR5095 | | | | | | | | |
| cg10049070 | 16 | NFATC3; NFATC3; NFATC3; | 2.89735E−13 | 1.16 | 0.71 | 0.54 | 0.88 | 76.98 | 66.17 | 10.81 |
| | | NFATC3 | | | | | | | | |
| cg01409709 | 2 | COL6A3; COL6A3; COL6A3; | 1.44425E−39 | 1.15 | 0.71 | 0.54 | 0.88 | 83.14 | 72.40 | 10.74 |
| | | COL6A3; COL6A3 | | | | | | | | |
| cg11941015 | 2 | C2orf88; C2orf88; C2orf88; C2orf88 | 3.23573E−15 | 1.13 | 0.71 | 0.54 | 0.88 | 84.12 | 74.24 | 9.88 |
| cg24706564 | 4 | MANBA | 1.36478E−12 | 1.14 | 0.71 | 0.54 | 0.88 | 80.63 | 70.89 | 9.75 |
| cg14282050 | 9 | MLLT3 | 1.04298E−14 | 1.13 | 0.71 | 0.54 | 0.88 | 83.79 | 74.05 | 9.74 |
| cg01477319 | 6 | PDSS2 | 9.0932E−12 | 1.14 | 0.71 | 0.54 | 0.88 | 79.30 | 69.60 | 9.69 |
| cg11973514 | 1 | AKT3; AKT3 | 8.59943E−14 | 1.13 | 0.71 | 0.54 | 0.88 | 82.98 | 73.35 | 9.63 |
| cg01390479 | 2 | HDAC4 | 2.09226E−11 | 1.14 | 0.71 | 0.54 | 0.88 | 79.37 | 69.85 | 9.52 |
| cg01974181 | 8 | STK3; STK3; STK3 | 2.82151E−12 | 1.13 | 0.71 | 0.54 | 0.88 | 82.09 | 72.82 | 9.27 |
| cg25406516 | 10 | GSTO2; GSTO2; GSTO2; GSTO2 | 8.76086E−12 | 1.13 | 0.71 | 0.54 | 0.88 | 81.77 | 72.62 | 9.15 |
| cg12639146 | 8 | RGS20; RGS20; RGS20; RGS20; | 5.10249E−11 | 1.12 | 0.71 | 0.54 | 0.88 | 81.65 | 72.80 | 8.85 |
| | | RGS20; RGS20; RGS20 | | | | | | | | |
| cg05179396 | 13 | KIAA0564 | 7.83632E−12 | 1.12 | 0.71 | 0.54 | 0.88 | 83.28 | 74.48 | 8.80 |
| cg05091003 | 2 | LCT | 3.51042E−14 | 1.11 | 0.71 | 0.54 | 0.88 | 86.81 | 78.15 | 8.66 |
| cg16708047 | 5 | TENM2 | 1.32585E−13 | 1.11 | 0.71 | 0.54 | 0.88 | 86.55 | 78.00 | 8.55 |
| cg21813265 | 1 | RCC2; RCC2 | 2.5376E−12 | 1.10 | 0.71 | 0.54 | 0.88 | 86.26 | 78.08 | 8.18 |
| cg25240363 | 17 | SSH2 | 3.97224E−11 | 1.10 | 0.71 | 0.54 | 0.88 | 85.70 | 77.81 | 7.89 |
| cg24500898 | 6 | EXOC2 | 1.00228E−13 | 1.09 | 0.71 | 0.54 | 0.88 | 90.39 | 83.07 | 7.32 |
| cg05761650 | 1 | DOCK7; DOCK7; DOCK7; DOCK7; | 5.9189E−11 | 1.09 | 0.71 | 0.54 | 0.88 | 88.49 | 81.46 | 7.03 |
| | | DOCK7 | | | | | | | | |
| cg26256263 | 1 | RUNX3; RUNX3 | 1.05873E−12 | 1.08 | 0.71 | 0.54 | 0.88 | 91.28 | 84.58 | 6.70 |
| cg17989581 | 5 | AHRR; PDCD6 | 1.53509E−37 | 1.07 | 0.71 | 0.54 | 0.88 | 95.18 | 89.35 | 5.83 |
| cg15757806 | 3 | RBMS3; RBMS3; RBMS3; RBMS3; | 9.67122E−18 | 0.80 | 0.71 | 0.54 | 0.88 | 62.58 | 77.99 | −15.41 |
| | | RBMS3 | | | | | | | | |
| cg12960352 | 8 | CLVS1 | 8.77723E−15 | 0.76 | 0.71 | 0.54 | 0.88 | 49.52 | 65.21 | −15.69 |
| cg20700869 | 8 | PTK2; PTK2; PTK2 | 4.13263E−14 | 1.43 | 0.71 | 0.54 | 0.88 | 44.26 | 30.87 | 13.39 |
| cg25855710 | 12 | KLRK1; LOC101928100; | 1.21176E−13 | 1.39 | 0.71 | 0.54 | 0.88 | 47.92 | 34.59 | 13.33 |
| | | KLRC4-KLRK1 | | | | | | | | |
| cg00629772 | 12 | PMCH | 4.972E−14 | 1.24 | 0.71 | 0.54 | 0.88 | 66.34 | 53.55 | 12.80 |
| cg09392801 | 16 | CAPN15 | 1.12949E−39 | 1.15 | 0.71 | 0.54 | 0.88 | 86.40 | 75.42 | 10.98 |
| cg20251156 | 12 | OR6C65 | 9.46015E−13 | 1.13 | 0.71 | 0.54 | 0.88 | 81.26 | 71.60 | 9.66 |
| cg09513309 | 1 | TAS1R1; TAS1R1; NOL9; TAS1R1; | 2.11618E−11 | 0.50 | 0.71 | 0.54 | 0.88 | 8.43 | 16.69 | −8.27 |
| | | TAS1R1 | | | | | | | | |
| cg08779777 | 7 | PIK3CG | 1.81424E−17 | 0.59 | 0.71 | 0.54 | 0.88 | 21.31 | 36.09 | −14.79 |
| cg27041724 | 9 | MLLT3 | 1.40297E−15 | 0.70 | 0.71 | 0.54 | 0.88 | 36.78 | 52.92 | −16.14 |
| cg17733194 | 12 | STRAP | 1.29615E−15 | 1.20 | 0.71 | 0.54 | 0.88 | 74.01 | 61.72 | 12.29 |
| cg18480675 | 3 | CMC1 | 5.12495E−13 | 1.22 | 0.71 | 0.54 | 0.88 | 68.02 | 55.88 | 12.14 |
| cg18048983 | 17 | C17orf104 | 7.40079E−15 | 1.18 | 0.71 | 0.54 | 0.88 | 76.95 | 65.46 | 11.49 |
| cg07799299 | 8 | ASAP1 | 3.63852E−15 | 1.17 | 0.71 | 0.54 | 0.88 | 77.42 | 65.95 | 11.47 |
| cg00360840 | 7 | C7orf38 | 1.18155E−13 | 1.18 | 0.71 | 0.54 | 0.88 | 75.34 | 64.04 | 11.30 |
| cg07537701 | 4 | BANK1; BANK1 | 4.02842E−11 | 1.20 | 0.71 | 0.54 | 0.88 | 67.65 | 56.39 | 11.26 |
| cg11809668 | 4 | KIAA0922; KIAA0922 | 1.97682E−15 | 1.17 | 0.71 | 0.54 | 0.88 | 78.97 | 67.77 | 11.20 |
| cg14926264 | 2 | SMYD1 | 4.57754E−12 | 1.18 | 0.71 | 0.54 | 0.88 | 73.18 | 62.21 | 10.97 |
| cg09563451 | 20 | PCNA | 3.34E−11 | 1.17 | 0.71 | 0.54 | 0.88 | 73.03 | 62.45 | 10.58 |
| cg09347495 | 6 | CLIC5 | 5.88103E−13 | 1.15 | 0.71 | 0.54 | 0.88 | 78.51 | 68.15 | 10.37 |
| cg12424781 | 7 | LOC100128317; LOC100128317 | 8.04395E−13 | 1.14 | 0.71 | 0.54 | 0.88 | 80.39 | 70.50 | 9.90 |
| cg11531783 | 7 | MLL5; MLL5 | 1.21647E−11 | 1.14 | 0.71 | 0.54 | 0.88 | 78.26 | 68.40 | 9.86 |
| cg08834517 | 10 | ZNF33B; ZNF33B; ZNF33B; | 1.32889E−11 | 1.14 | 0.71 | 0.54 | 0.88 | 79.03 | 69.34 | 9.68 |
| | | ZNF33B; ZNF33B; ZNF33B; | | | | | | | | |
| | | ZNF33B; ZNF33B; ZNF33B; | | | | | | | | |
| | | ZNF33B; ZNF33B; ZNF33B | | | | | | | | |
| cg18671377 | 4 | LOC401127 | 1.265E−11 | 1.13 | 0.71 | 0.54 | 0.88 | 80.03 | 70.56 | 9.47 |
| cg07615234 | 1 | RPS6KC1; RPS6KC1; RPS6KC1; | 2.37849E−11 | 1.13 | 0.71 | 0.54 | 0.88 | 79.81 | 70.41 | 9.40 |
| | | RPS6KC1; RPS6KC1; RPS6KC1 | | | | | | | | |
| cg02765475 | 5 | SLC38A9; SLC38A9; SLC38A9; | 7.82685E−15 | 1.12 | 0.71 | 0.54 | 0.88 | 85.21 | 75.83 | 9.38 |
| | | SLC38A9; SLC38A9 | | | | | | | | |
| cg14950428 | 5 | ALDH7A1; ALDH7A1; ALDH7A1 | 1.36997E−11 | 1.13 | 0.71 | 0.54 | 0.88 | 80.71 | 71.40 | 9.31 |
| cg21220670 | 6 | ETV7; ETV7; ETV7; ETV7; ETV7; | 8.35426E−12 | 1.13 | 0.71 | 0.54 | 0.88 | 81.32 | 72.06 | 9.26 |
| | | ETV7; ETV7; ETV7 | | | | | | | | |
| cg13048261 | 8 | MIR597; TNKS | 6.24936E−39 | 1.12 | 0.71 | 0.54 | 0.88 | 86.78 | 77.53 | 9.25 |
| cg21346855 | 14 | MIR544A; MIR381HG; MIR655 | 1.61968E−11 | 1.12 | 0.71 | 0.54 | 0.88 | 81.80 | 72.77 | 9.03 |
| cg03182373 | 17 | FAM104A; FAM104A; FAM104A; | 5.99756E−12 | 1.12 | 0.71 | 0.54 | 0.88 | 82.57 | 73.55 | 9.02 |
| | | FAM104A; FAM104A | | | | | | | | |
| cg05736824 | 10 | INPP5A | 2.71317E−11 | 1.12 | 0.71 | 0.54 | 0.88 | 82.04 | 73.17 | 8.88 |
| cg02724868 | 3 | PODXL2 | 1.08833E−11 | 1.12 | 0.71 | 0.54 | 0.88 | 82.76 | 73.90 | 8.87 |
| cg17789762 | 3 | XRN1; XRN1; XRN1; XRN1 | 8.88868E−14 | 1.11 | 0.71 | 0.54 | 0.88 | 85.79 | 76.96 | 8.83 |
| cg11230471 | 17 | SLFN12 | 2.77754E−11 | 1.12 | 0.71 | 0.54 | 0.88 | 83.09 | 74.47 | 8.62 |
| cg02292450 | 8 | CSMD3; CSMD3; CSMD3 | 6.66499E−13 | 1.11 | 0.71 | 0.54 | 0.88 | 86.09 | 77.65 | 8.44 |
| cg14621978 | 5 | NR3C1; NR3C1; NR3C1; NR3C1; | 3.353E−11 | 1.11 | 0.71 | 0.54 | 0.88 | 83.90 | 75.52 | 8.38 |
| | | NR3C1; NR3C1; NR3C1; NR3C1; | | | | | | | | |
| | | NR3C1; NR3C1; NR3C1; NR3C1; | | | | | | | | |
| | | NR3C1; NR3C1; NR3C1 | | | | | | | | |
| cg14077073 | 10 | ST8SIA6 | 1.93141E−13 | 1.11 | 0.71 | 0.54 | 0.88 | 86.99 | 78.63 | 8.36 |
| cg21245492 | 15 | IGF1R | 4.86653E−11 | 1.10 | 0.71 | 0.54 | 0.88 | 85.21 | 77.23 | 7.98 |
| cg14844138 | 1 | LZIC | 1.95637E−11 | 1.10 | 0.71 | 0.54 | 0.88 | 86.70 | 78.98 | 7.73 |
| cg26975648 | 2 | SP140; SP140; SP140; SP140 | 2.78871E−11 | 1.09 | 0.71 | 0.54 | 0.88 | 87.21 | 79.68 | 7.53 |
| cg22338356 | 8 | PRKDC; PRKDC | 1.60418E−11 | 1.08 | 0.71 | 0.54 | 0.88 | 89.53 | 82.62 | 6.91 |
| cg18005217 | 10 | INPP5A | 1.4772E−13 | 1.07 | 0.71 | 0.54 | 0.88 | 92.94 | 86.61 | 6.33 |
| cg05176970 | 17 | NXN | 2.15366E−42 | 1.26 | 0.71 | 0.54 | 0.87 | 81.30 | 64.50 | 16.79 |
| cg19504888 | 2 | MFF | 1.86085E−41 | 1.28 | 0.71 | 0.54 | 0.87 | 68.90 | 54.02 | 14.88 |
| cg27454408 | 16 | SNTB2 | 2.34377E−14 | 1.21 | 0.71 | 0.54 | 0.87 | 70.71 | 58.33 | 12.38 |
| cg00036599 | 1 | C1orf54 | 2.72845E−40 | 1.19 | 0.71 | 0.54 | 0.87 | 76.76 | 64.38 | 12.37 |
| cg08465505 | 4 | RNF150 | 2.73997E−40 | 1.19 | 0.71 | 0.54 | 0.87 | 76.20 | 63.84 | 12.37 |
| cg11726229 | 3 | GABRR3 | 6.70726E−13 | 1.22 | 0.71 | 0.54 | 0.87 | 67.90 | 55.80 | 12.10 |
| cg19887462 | 3 | PLXNB1; PLXNB1 | 1.52647E−11 | 1.24 | 0.71 | 0.54 | 0.87 | 61.47 | 49.44 | 12.03 |
| cg09341389 | 8 | RIMS2; RIMS2; RIMS2 | 9.78929E−12 | 1.24 | 0.71 | 0.54 | 0.87 | 63.02 | 51.01 | 12.02 |
| cg25689728 | 4 | SMAD1-AS2; SMAD1; SMAD1 | 1.75164E−11 | 1.21 | 0.71 | 0.54 | 0.87 | 66.56 | 55.00 | 11.56 |
| cg25586973 | 21 | KRTAP21-2 | 1.78674E−11 | 1.21 | 0.71 | 0.54 | 0.87 | 66.84 | 55.31 | 11.53 |
| cg12797771 | 1 | NME7; NME7 | 1.06124E−39 | 1.16 | 0.71 | 0.54 | 0.87 | 80.40 | 69.35 | 11.05 |
| cg11261509 | 18 | ALPK2 | 3.15266E−11 | 1.17 | 0.71 | 0.54 | 0.87 | 72.99 | 62.39 | 10.60 |
| cg03603542 | 3 | C3orf59 | 1.2917E−14 | 1.15 | 0.71 | 0.54 | 0.87 | 80.47 | 69.89 | 10.58 |
| cg21822551 | 13 | ZC3H13 | 2.39224E−39 | 1.14 | 0.71 | 0.54 | 0.87 | 84.27 | 74.04 | 10.23 |
| cg00984787 | 5 | CENPK | 2.27377E−11 | 1.13 | 0.71 | 0.54 | 0.87 | 79.84 | 70.43 | 9.41 |
| cg01321962 | 6 | ESR1; ESR1 | 2.81177E−14 | 1.12 | 0.71 | 0.54 | 0.87 | 84.74 | 75.42 | 9.31 |
| cg26897313 | 15 | GCOM1; GCOM1; GCOM1; | 3.21995E−13 | 1.12 | 0.71 | 0.54 | 0.87 | 84.37 | 75.33 | 9.04 |
| | | GCOM1 | | | | | | | | |
| cg03262885 | 7 | PTPRN2; PTPRN2; PTPRN2 | 1.43363E−13 | 1.12 | 0.71 | 0.54 | 0.87 | 85.24 | 76.31 | 8.92 |
| cg16301599 | 17 | HRNBP3 | 1.20734E−38 | 1.11 | 0.71 | 0.54 | 0.87 | 89.52 | 80.95 | 8.57 |
| cg02886541 | 14 | SIP1; SIP1; SIP1 | 3.38182E−11 | 1.11 | 0.71 | 0.54 | 0.87 | 83.31 | 74.78 | 8.53 |
| cg27289662 | 15 | OCA2 | 3.58926E−12 | 1.11 | 0.71 | 0.54 | 0.87 | 86.04 | 77.86 | 8.19 |
| cg12876808 | 1 | NEXN; NEXN | 3.17961E−12 | 1.09 | 0.71 | 0.54 | 0.87 | 88.60 | 81.16 | 7.44 |
| cg12571879 | 19 | C19orf59; C19orf59 | 9.32419E−13 | 0.48 | 0.71 | 0.54 | 0.87 | 8.02 | 16.72 | −8.71 |
| cg24272305 | 1 | KCNT2; KCNT2; KCNT2 | 6.52324E−16 | 0.54 | 0.71 | 0.54 | 0.87 | 14.17 | 26.26 | −12.09 |
| cg17766351 | 8 | PTK2 | 1.21085E−40 | 1.22 | 0.70 | 0.53 | 0.87 | 71.91 | 58.76 | 13.15 |
| cg08414984 | 6 | RIMS1; RIMS1; RIMS1; RIMS1; | 1.17434E−13 | 1.28 | 0.70 | 0.53 | 0.87 | 60.76 | 47.65 | 13.11 |
| | | RIMS1 | | | | | | | | |
| cg06938133 | 17 | WDR45L | 2.18617E−12 | 1.25 | 0.70 | 0.53 | 0.87 | 62.06 | 49.64 | 12.42 |
| cg19064941 | 22 | MTMR3; MTMR3; MTMR3 | 8.9904E−13 | 1.22 | 0.70 | 0.53 | 0.87 | 66.48 | 54.27 | 12.20 |
| cg22893924 | 2 | EML6 | 3.70394E−40 | 1.19 | 0.70 | 0.53 | 0.87 | 76.82 | 64.74 | 12.08 |
| cg00076256 | 7 | RBM28; RBM28 | 3.70455E−40 | 1.19 | 0.70 | 0.53 | 0.87 | 77.31 | 65.23 | 12.08 |
| cg24034923 | 1 | ANGEL2 | 5.93997E−14 | 1.19 | 0.70 | 0.53 | 0.87 | 73.12 | 61.30 | 11.82 |
| cg07914567 | 1 | MIA3; MIA3 | 5.3357E−14 | 1.18 | 0.70 | 0.53 | 0.87 | 75.13 | 63.65 | 11.49 |
| cg15761531 | 3 | GLT8D1; GLT8D1; GLT8D1; | 2.67769E−12 | 1.19 | 0.70 | 0.53 | 0.87 | 71.44 | 60.10 | 11.35 |
| | | GLT8D1 | | | | | | | | |
| cg07666291 | 20 | XRN2 | 9.17816E−40 | 1.16 | 0.70 | 0.53 | 0.87 | 81.49 | 70.30 | 11.19 |
| cg04593745 | 2 | DYTN | 1.81173E−12 | 1.18 | 0.70 | 0.53 | 0.87 | 73.19 | 62.04 | 11.15 |
| cg10260477 | 7 | POM121L12 | 1.30814E−12 | 1.17 | 0.70 | 0.53 | 0.87 | 74.87 | 63.94 | 10.93 |
| cg01014371 | 16 | CBFA2T3 | 3.18591E−11 | 1.18 | 0.70 | 0.53 | 0.87 | 72.02 | 61.27 | 10.74 |
| cg04970438 | 10 | TAF3 | 1.07877E−11 | 1.16 | 0.70 | 0.53 | 0.87 | 74.84 | 64.32 | 10.52 |
| cg07201934 | 7 | TMEM168; TMEM168; TMEM168 | 2.77254E−12 | 1.16 | 0.70 | 0.53 | 0.87 | 76.94 | 66.54 | 10.40 |
| cg19773249 | 7 | ACTR3C; ACTR3C | 1.24137E−13 | 1.15 | 0.70 | 0.53 | 0.87 | 79.67 | 69.28 | 10.39 |
| cg10504568 | 9 | DENND1A; DENND1A | 6.7372E−12 | 1.16 | 0.70 | 0.53 | 0.87 | 76.58 | 66.29 | 10.30 |
| cg19032370 | 1 | EVI5; EVI5 | 5.90367E−11 | 1.16 | 0.70 | 0.53 | 0.87 | 74.28 | 64.02 | 10.26 |
| cg02000823 | 14 | MIS18BP1 | 4.34723E−12 | 1.15 | 0.70 | 0.53 | 0.87 | 77.23 | 66.98 | 10.26 |
| cg01453814 | 19 | SLC6A16 | 2.39789E−14 | 1.14 | 0.70 | 0.53 | 0.87 | 81.75 | 71.60 | 10.15 |
| cg21931078 | 19 | CDC42EP5 | 1.68603E−12 | 1.15 | 0.70 | 0.53 | 0.87 | 78.88 | 68.78 | 10.10 |
| cg16612083 | 1 | OLFM3; OLFM3; OLFM3; | 5.09694E−11 | 1.14 | 0.70 | 0.53 | 0.87 | 78.24 | 68.67 | 9.57 |
| | | OLFM3; OLFM3; OLFM3 | | | | | | | | |
| cg10847048 | 1 | PRDM2; PRDM2; PRDM2 | 1.55568E−12 | 1.13 | 0.70 | 0.53 | 0.87 | 82.06 | 72.67 | 9.38 |
| cg27296835 | 17 | USP32 | 5.03211E−12 | 1.12 | 0.70 | 0.53 | 0.87 | 82.44 | 73.36 | 9.08 |
| cg11742016 | 4 | ATP8A1; ATP8A1 | 1.79118E−12 | 1.12 | 0.70 | 0.53 | 0.87 | 84.35 | 75.58 | 8.77 |
| cg14978440 | 12 | GPR19 | 2.13654E−11 | 1.11 | 0.70 | 0.53 | 0.87 | 83.43 | 74.85 | 8.58 |
| cg21923842 | 15 | ASB7; ASB7 | 5.93584E−15 | 1.10 | 0.70 | 0.53 | 0.87 | 88.76 | 80.55 | 8.21 |
| cg14667570 | 7 | MKLN1 | 3.80903E−11 | 1.10 | 0.70 | 0.53 | 0.87 | 86.68 | 79.05 | 7.63 |
| cg02084828 | 5 | SEMA6A | 4.59805E−11 | 1.08 | 0.70 | 0.53 | 0.87 | 90.56 | 84.13 | 6.43 |
| cg08463929 | 6 | FBXO5; FBXO5 | 4.87874E−14 | 0.44 | 0.70 | 0.53 | 0.87 | 7.04 | 15.83 | −8.80 |
| cg23393334 | 13 | FNDC3A; FNDC3A; FNDC3A; | 4.06346E−15 | 0.46 | 0.70 | 0.53 | 0.87 | 8.25 | 17.91 | −9.66 |
| | | FNDC3A; FNDC3A | | | | | | | | |
| cg16901197 | 14 | LINC00911; LINC00911 | 5.58375E−11 | 0.66 | 0.70 | 0.53 | 0.87 | 23.66 | 35.60 | −11.93 |
| cg20474604 | 9 | PTPRD | 4.97626E−12 | 0.63 | 0.70 | 0.53 | 0.87 | 20.77 | 32.77 | −12.00 |
| cg01094580 | 2 | GLS | 9.78722E−41 | 1.23 | 0.70 | 0.53 | 0.87 | 71.78 | 58.43 | 13.35 |
| cg25398258 | 3 | EOGT; EOGT; EOGT | 1.06272E−14 | 1.24 | 0.70 | 0.53 | 0.87 | 66.77 | 53.72 | 13.05 |
| cg10674583 | 7 | SDHAF3 | 1.34682E−40 | 1.22 | 0.70 | 0.53 | 0.87 | 72.93 | 59.89 | 13.05 |
| cg18442469 | 8 | C8orf76; ZHX1-C8orf76 | 6.3E−14 | 1.25 | 0.70 | 0.53 | 0.87 | 65.16 | 52.29 | 12.87 |
| cg25255271 | 19 | ZFP30 | 2.48241E−40 | 1.19 | 0.70 | 0.53 | 0.87 | 77.22 | 64.76 | 12.46 |
| cg09882297 | 18 | LINC00305; LOC284294 | 7.0031E−13 | 1.21 | 0.70 | 0.53 | 0.87 | 68.42 | 56.40 | 12.03 |
| cg10826401 | 13 | NEK3; NEK3; NEK3; NEK3 | 3.81292E−12 | 1.22 | 0.70 | 0.53 | 0.87 | 65.56 | 53.56 | 12.00 |
| cg17674811 | 2 | TIA1; TIA1 | 5.22975E−40 | 1.17 | 0.70 | 0.53 | 0.87 | 80.40 | 68.66 | 11.74 |
| cg11448063 | 7 | SNX10; SNX10; SNX10 | 3.83302E−15 | 1.17 | 0.70 | 0.53 | 0.87 | 78.26 | 66.92 | 11.33 |
| cg14322719 | 9 | RALGPS1 | 4.16046E−15 | 1.16 | 0.70 | 0.53 | 0.87 | 79.10 | 67.99 | 11.11 |
| cg26552907 | 6 | MDC1 | 1.42147E−13 | 1.17 | 0.70 | 0.53 | 0.87 | 76.45 | 65.40 | 11.05 |
| cg21619340 | 4 | SHROOM3 | 9.1646E−13 | 1.17 | 0.70 | 0.53 | 0.87 | 75.44 | 64.54 | 10.90 |
| cg13018643 | 7 | EEPD1 | 2.24645E−15 | 1.16 | 0.70 | 0.53 | 0.87 | 80.63 | 69.77 | 10.86 |
| cg15171098 | 8 | TUSC3; TUSC3 | 4.84377E−14 | 1.16 | 0.70 | 0.53 | 0.87 | 78.62 | 67.83 | 10.79 |
| cg09708852 | 7 | HUS1; HUS1 | 1.40926E−39 | 1.15 | 0.70 | 0.53 | 0.87 | 81.47 | 70.70 | 10.76 |
| cg22222092 | 13 | BIVM; KDELC1; BIVM | 6.33907E−13 | 1.16 | 0.70 | 0.53 | 0.87 | 77.65 | 67.11 | 10.54 |
| cg05772550 | 11 | CCDC81; CCDC81 | 6.20136E−12 | 1.16 | 0.70 | 0.53 | 0.87 | 75.89 | 65.45 | 10.44 |
| cg15158031 | 19 | ARID3A | 8.97241E−14 | 1.15 | 0.70 | 0.53 | 0.87 | 79.71 | 69.28 | 10.43 |
| cg20242129 | 17 | NSF | 1.76462E−11 | 1.16 | 0.70 | 0.53 | 0.87 | 75.06 | 64.67 | 10.38 |
| cg08339787 | 3 | ARL14 | 4.34834E−11 | 1.16 | 0.70 | 0.53 | 0.87 | 74.76 | 64.51 | 10.25 |
| cg16438602 | 12 | SP7 | 4.51491E−11 | 1.16 | 0.70 | 0.53 | 0.87 | 75.30 | 65.15 | 10.15 |
| cg23462990 | 7 | GPR141 | 4.63926E−11 | 1.15 | 0.70 | 0.53 | 0.87 | 76.00 | 65.98 | 10.02 |
| cg16971976 | 6 | MDN1 | 6.15422E−11 | 1.13 | 0.70 | 0.53 | 0.87 | 79.43 | 70.13 | 9.30 |
| cg23581666 | 5 | ANKH | 6.12529E−13 | 1.12 | 0.70 | 0.53 | 0.87 | 84.69 | 75.85 | 8.85 |
| cg27425463 | 11 | SOX6; SOX6; SOX6; SOX6 | 2.2274E−11 | 1.12 | 0.70 | 0.53 | 0.87 | 82.38 | 73.55 | 8.83 |
| cg12647415 | 10 | FRMD4A | 1.22169E−12 | 1.11 | 0.70 | 0.53 | 0.87 | 85.05 | 76.41 | 8.64 |
| cg19285539 | 22 | SEPT3; SEPT3; WBP2NL | 4.30027E−11 | 1.11 | 0.70 | 0.53 | 0.87 | 83.74 | 75.36 | 8.38 |
| cg14306118 | 15 | RORA; RORA; RORA; RORA | 3.77974E−11 | 1.11 | 0.70 | 0.53 | 0.87 | 84.04 | 75.71 | 8.33 |
| cg06161915 | 7 | SND1 | 1.3441E−11 | 1.09 | 0.70 | 0.53 | 0.87 | 87.49 | 79.93 | 7.56 |
| cg11449588 | 5 | MTMR12; MTMR12; MTMR12 | 2.58781E−11 | 1.09 | 0.70 | 0.53 | 0.87 | 87.25 | 79.72 | 7.53 |
| cg13162749 | 15 | MTMR15; MTMR15; MTMR15; | 1.35749E−11 | 1.09 | 0.70 | 0.53 | 0.87 | 88.52 | 81.27 | 7.25 |
| | | MTMR15 | | | | | | | | |
| cg04138181 | 16 | PLK1 | 2.8951E−15 | 0.51 | 0.70 | 0.53 | 0.87 | 11.22 | 22.04 | −10.82 |
| cg17997673 | 1 | OSBPL9; OSBPL9; OSBPL9; | 3.99136E−16 | 0.75 | 0.70 | 0.53 | 0.87 | 48.56 | 64.99 | −16.43 |
| | | OSBPL9; OSBPL9 | | | | | | | | |
| cg15841069 | 2 | ARHGAP15 | 6.39811E−16 | 1.29 | 0.70 | 0.52 | 0.87 | 61.99 | 47.93 | 14.06 |
| cg00914121 | 8 | MMP16 | 1.75938E−13 | 1.21 | 0.70 | 0.52 | 0.87 | 70.12 | 58.04 | 12.08 |
| cg10539170 | 14 | SCFD1; SCFD1; SCFD1; SCFD1; | 5.02004E−14 | 1.17 | 0.70 | 0.52 | 0.87 | 77.18 | 66.09 | 11.09 |
| | | SCFD1; SCFD1 | | | | | | | | |
| cg14618360 | 13 | ABCC4; ABCC4; ABCC4; ABCC4 | 3.0335E−11 | 1.17 | 0.70 | 0.52 | 0.87 | 72.20 | 61.47 | 10.73 |
| cg27149567 | 21 | PCBP3; PCBP3 | 7.64532E−12 | 1.16 | 0.70 | 0.52 | 0.87 | 75.70 | 65.27 | 10.44 |
| cg08474576 | 2 | CRIM1 | 5.61221E−12 | 1.14 | 0.70 | 0.52 | 0.87 | 78.97 | 69.12 | 9.86 |
| cg07626409 | 16 | ZKSCAN2 | 1.28565E−11 | 1.13 | 0.70 | 0.52 | 0.87 | 80.93 | 71.66 | 9.27 |
| cg21747652 | 5 | CTNNA1 | 3.33997E−11 | 1.12 | 0.70 | 0.52 | 0.87 | 82.21 | 73.42 | 8.80 |
| cg06670685 | 9 | NUP214 | 3.47727E−14 | 1.10 | 0.70 | 0.52 | 0.87 | 88.11 | 79.87 | 8.25 |
| cg18693889 | 1 | RNPC3; AMY2B | 1.20071E−14 | 1.30 | 0.70 | 0.52 | 0.87 | 58.95 | 45.29 | 13.67 |
| cg01543184 | 17 | MAFG; MAFG | 6.08395E−14 | 1.44 | 0.70 | 0.52 | 0.87 | 43.73 | 30.45 | 13.29 |
| cg25455317 | 9 | NFIB; NFIB; NFIB; NFIB | 5.88042E−14 | 1.26 | 0.70 | 0.52 | 0.87 | 63.82 | 50.80 | 13.02 |
| cg05163390 | 4 | AFF1; AFF1 | 1.65829E−12 | 1.28 | 0.70 | 0.52 | 0.87 | 58.22 | 45.52 | 12.70 |
| cg04785555 | 4 | FNIP2 | 3.43708E−40 | 1.18 | 0.70 | 0.52 | 0.87 | 80.75 | 68.60 | 12.15 |
| cg02252928 | 6 | OSTM1 | 6.16096E−40 | 1.17 | 0.70 | 0.52 | 0.87 | 80.26 | 68.67 | 11.58 |
| cg02286968 | 5 | TERT; TERT | 6.8531E−12 | 1.20 | 0.70 | 0.52 | 0.87 | 68.91 | 57.41 | 11.50 |
| cg25306198 | 5 | DCTN4; DCTN4; DCTN4 | 7.59064E−40 | 1.17 | 0.70 | 0.52 | 0.87 | 79.80 | 68.42 | 11.38 |
| cg21218433 | 2 | DAW1 | 4.23172E−15 | 1.16 | 0.70 | 0.52 | 0.87 | 79.35 | 68.29 | 11.07 |
| cg21161367 | 1 | VAV3 | 1.59605E−39 | 1.15 | 0.70 | 0.52 | 0.87 | 82.33 | 71.69 | 10.64 |
| cg10794257 | 7 | HOXA3; HOXA3; HOXA3 | 1.73593E−13 | 1.15 | 0.70 | 0.52 | 0.87 | 78.73 | 68.19 | 10.54 |
| cg06008844 | 10 | ANK3; ANK3; ANK3; ANK3 | 2.27399E−11 | 1.16 | 0.70 | 0.52 | 0.87 | 74.49 | 64.07 | 10.43 |
| cg17498635 | 11 | SNORA23; IPO7 | 2.0158E−13 | 1.15 | 0.70 | 0.52 | 0.87 | 79.51 | 69.17 | 10.34 |
| cg04894116 | 7 | C7orf50; C7orf50; C7orf50 | 3.97125E−12 | 1.15 | 0.70 | 0.52 | 0.87 | 77.25 | 66.98 | 10.27 |
| cg24155984 | 5 | MAST4; MAST4; MAST4 | 3.30366E−11 | 1.14 | 0.70 | 0.52 | 0.87 | 78.98 | 69.46 | 9.51 |
| cg26402799 | 12 | ADAMTS20 | 2.63258E−14 | 1.12 | 0.70 | 0.52 | 0.87 | 85.63 | 76.57 | 9.06 |
| cg06800293 | 5 | TRIO | 1.23707E−11 | 1.11 | 0.70 | 0.52 | 0.87 | 83.81 | 75.23 | 8.58 |
| cg24000516 | 18 | DLGAP1; DLGAP1; DLGAP1; | 3.6976E−12 | 1.10 | 0.70 | 0.52 | 0.87 | 87.02 | 79.13 | 7.90 |
| | | DLGAP1; DLGAP1; DLGAP1; | | | | | | | | |
| | | DLGAP1; DLGAP1; DLGAP1 | | | | | | | | |
| cg21089065 | 18 | NFATC1; NFATC1; NFATC1; | 2.53523E−38 | 1.09 | 0.70 | 0.52 | 0.87 | 90.89 | 83.10 | 7.79 |
| | | NFATC1 | | | | | | | | |
| cg07591090 | 10 | TCF7L2; TCF7L2; TCF7L2; | 2.74282E−11 | 1.09 | 0.70 | 0.52 | 0.87 | 87.95 | 80.63 | 7.31 |
| | | TCF7L2; TCF7L2; TCF7L2 | | | | | | | | |
| cg24924563 | 2 | TGOLN2 | 6.06197E−15 | 0.47 | 0.70 | 0.52 | 0.87 | 8.72 | 18.50 | −9.78 |
| cg03209103 | 7 | SGCE; SGCE; PEG10; SGCE; | 1.83222E−16 | 0.54 | 0.70 | 0.52 | 0.87 | 14.21 | 26.53 | −12.33 |
| | | PEG10 | | | | | | | | |
| cg21501207 | 1 | SH2D1B | 4.12196E−14 | 0.65 | 0.70 | 0.52 | 0.87 | 25.96 | 40.08 | −14.12 |
| cg02807450 | 11 | MTMR2; MTMR2; MTMR2; | 7.18417E−27 | 0.62 | 0.70 | 0.52 | 0.87 | 35.26 | 56.41 | −21.16 |
| | | MTMR2 | | | | | | | | |

TABLE 2
Artificial Intelligence and Circulating cfF DNA Prediction of CHD*

| | SVM | GLM | PAM | RF | LDA | DL |
|---|---|---|---|---|---|---|
| AUC | 0.9700 | 0.9400 | 0.9300 | 0.9800 | 0.9200 | 0.9400 |
| 95% Cl | (0.8770-1) | (0.8240-1) | (0.8300-1) | (0.8310-1) | (0.7240-1) | (0.8400.1) |
| SENSITIVITY | 0.9800 | 0.9200 | 0.8700 | 0.9380 | 0.9700 | 0.9300 |
| SPEC | 0.9400 | 0.8700 | 0.9200 | 0.9320 | 0.9300 | 0.9400 |

*Individual CpG markers chosen based on methylation difference of ≥5% and individual AUC for CHD Detection ≥0.70

### Individual Predictors in Decreasing Order of Contribution:

SVM: cg04761177, cg21431091, cg01263077, cg09853933, cg27142059
GLM: cg24479965, cg01094213, cg22467129, cg24748945, cg01949461
PAM: cg27142059, cg09386284, cg16551159, cg04761177, cg01263077
RF: cg04761177, cg16551159, cg14957943, cg06978680, cg12592721
LDA: cg04761177, cg27142059, cg18073832, cg25731807, cg03790075
DL: cg04761177, cg21431091, cg01263077, cg09853933, cg27142059

TABLE 3
Artificial Intelligence and Circulating cfF DNA,
Clinical and Demographic Prediction of CHD*

| | SVM | GLM | PAM | RF | LDA | DL |
|---|---|---|---|---|---|---|
| AUC | 0.9680 | 0.9450 | 0.9370 | 0.9670 | 0.9120 | 0.9340 |
| 95% Cl | (0.8770-1) | (0.8240-1) | (0.8300-1) | (0.8310-1) | (0.7240-1) | (0.8340-1) |
| SENSITIVITY | 0.9700 | 0.9100 | 0.8800 | 0.9280 | 0.9600 | 0.9300 |
| SPEC | 0.9200 | 0.8600 | 0.9100 | 0.9220 | 0.9100 | 0.9200 |

*Individual CpG markers chosen based on methylation difference of ≥5% and individual AUC for CHD Detection ≥0.70

### Individual Predictors in Decreasing Order of Contribution:

SVM: cg04761177, cg21431091, cg01263077, cg09853933, cg27142059
GLM: cg24479965, cg01094213, cg22467129, cg24748945, cg01949461
PAM: cg27142059, cg09386284, cg16551159, cg04761177, cg01263077
RF: cg04761177, cg16551159, cg14957943, cg06978680, cg12592721
LDA: cg04761177, cg27142059, cg18073832, cg25731807, cg03790075
DL: cg04761177, cg21431091, cg01263077, cg09853933, cg27142059

TABLE 4
Artificial Intelligence, Circulating cfF DNA Meeting
Stringent Criteria* Prediction of CHD*

| | SVM | GLM | PAM | RF | LDA | DL |
|---|---|---|---|---|---|---|
| AUC | 0.9300 | 0.9270 | 0.9370 | 0.9170 | 0.8970 | 0.9290 |
| 95% Cl | (0.7770-1) | (0.7270-1) | (0.7370-1) | (0.7170-1) | (0.6970-1) | (0.8290-1) |
| SENSITIVITY | 0.8800 | 0.8900 | 0.9000 | 0.9000 | 0.8890 | 0.9000 |
| SPEC | 0.8730 | 0.9030 | 0.8730 | 0.8900 | 0.8700 | 0.9190 |

*Individual CpG markers limited to those meeting stringent threshold for GWAS studies (p-value ≤ 5 × 10⁻⁸)

### Individual Predictors in Decreasing Order of Contribution:

SVM: cg04761177, cg15277677, cg03790075, cg04626875, cg11782260
GLM: cg13598434, cg05349624, cg10259004, cg04761177, cg11196182
PAM: cg18198743, cg08316054, cg02394812, cg00280345, cg08052226
RF: cg04761177, cg24412848 0.62684, cg01637563, cg17720707, cg05349624
LDA: cg03790075, cg04761177, cg05349624, cg14809932, cg01637563
DL: cg13598434, cg05349624, cg10259004, cg04761177, cg11196182

TABLE 5
Artificial Intelligence, Circulating cfF DNA Meeting Stringent
Criteria*, Clinical and Demographic Prediction of CHD

| | SVM | GLM | PAM | RF | LDA | DL |
|---|---|---|---|---|---|---|
| AUC | 0.9280 | 0.9230 | 0.9310 | 0.9120 | 0.8940 | 0.9270 |
| 95% Cl | (0.7770-1) | (0.7270-1) | (0.7370-1) | (0.7170-1) | (0.6970-1) | (0.8290-1) |
| SENSITIVITY | 0.8900 | 0.8700 | 0.8900 | 0.9100 | 0.8790 | 0.9100 |
| SPEC | 0.8830 | 0.8930 | 0.8830 | 0.8800 | 0.8800 | 0.9090 |

*Individual CpG markers limited to those meeting stringent threshold for GWAS studies (p-value ≤ 5 × 10⁻⁸)

### Individual Predictors in Decreasing Order of Contribution:

SVM: cg04761177, cg15277677, cg03790075, cg04626875, cg11782260
GLM: cg13598434, cg05349624, cg10259004, cg04761177, cg11196182
PAM: cg18198743, cg08316054, cg02394812, cg00280345, cg08052226
RF: cg04761177, cg24412848 0.62684, cg01637563, cg17720707, cg05349624
LDA: cg03790075, cg04761177, cg05349624, cg14809932, cg01637563
DL: cg13598434, cg05349624, cg10259004, cg04761177, cg11196182

TABLE 6
Artificial Intelligence and Circulating cfF DNA* prediction of CHD

| | SVM | GLM | PAM | RF | LDA | DL |
|---|---|---|---|---|---|---|
| AUC | 0.9400 | 0.9540 | 0.9400 | 0.9700 | 0.9300 | 0.9480 |
| 95% Cl | (0.7200-1) | (0.7740-1) | (0.7950-1) | (0.7770-1) | (0.7370-1) | (0.8010-1) |
| SENSITIVITY | 0.9200 | 0.9060 | 0.9060 | 0.9020 | 0.9200 | 0.9200 |
| SPEC | 0.9100 | 0.9090 | 0.9030 | 0.9080 | 0.9100 | 0.9050 |

*Limited to Individual CpG markers with ≥1.5 Fold methylation change in CHD versus controls

### Individual Predictors in Decreasing Order of Contribution:

SVM: cg09493833, cg27563174, cg22752533, cg05279901, cg03741571
GLM: cg19803352, cg24479965, cg07287606, cg22467129, cg24509810
PAM: cg27142059, cg13598434, cg14200609, cg09493833, cg20360734
RF: cg27142059, cg26078733, cg14200609, cg23273875, cg24509810
LDA: cg27142059, cg09493833, cg19803352, cg17485454, cg07287606
DL: cg27142059, cg26078733, cg14200609, cg23273875, cg24509810

Microarray. Differential methylation can be analyzed using a microarray system. Nucleic acids can be linked to chips, such as microchips. See, for example, U.S. Pat. Nos. 5,143,854; 6,087,112; 5,215,882; 5,707,807; 5,807,522; 5,958,342; 5,994,076; 6,004,755; 6,048,695; 6,060,240; 6,090,556; and 6,040,138. Binding to nucleic acids on microarrays can be detected by scanning the microarray with a variety of laser or charge coupled device (CCD)-based scanners, and extracting features with software packages, for example, Imagene (Biodiscovery, Hawthorne, Calif.), Feature Extraction Software (Agilent), Scanalyze (Eisen, M. 1999. SCANALYZE User Manual; Stanford Univ., Stanford, Calif. Ver 2.3.2.), or GenePix (Axon Instruments). A full panel of loci would include one or more genomic loci listed in Tables 1-6 that have been shown individually to be potentially clinically useful tests AUC0.70.

Kits. Kits for predicting and diagnosing CHD based on methylation of CpG loci on nucleic acids are described. The kits can include the components for extracting nucleic acids including DNA and RNA from the biological sample, the components of a microarray system, and/or for analysis of the differentially methylated genomic sites.

Biomarker detection of CHD as described herein can lead to the early and accurate diagnosis and thus facilitate the management objectives outlined by the CDC. Given the evidence that a significant percentage even a majority of major CHD cases remain undiagnosed, accurate biomarkers are a critical necessary complement to any effective treatment strategy.

Methods disclosed herein include predicting, detecting, or diagnosing CHD and/or calculating risk or disposition to developing CHD. The methods described herein can be used in the prevention and/or treatment (including mitigating or alleviating symptoms) of patients at an early stage to prevent death or the development of severe symptoms associated with CHD. Subjects or patients in need of (in need thereof) predicting, diagnosing, and/or treating are subjects that may have CHD and need to be diagnosed and treated.

As will be understood by one of ordinary skill in the art, each embodiment disclosed herein can comprise, consist essentially of, or consist of its particular stated element, step, ingredient, or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. As an example, steps that do not affect the detection, prediction, diagnosis of CHD, or do not affect the prevention or treating of CHD of a patient.

In addition, unless otherwise indicated, numbers expressing quantities of ingredients, constituents, reaction conditions and so forth used in the specification and claims are to be understood as being modified by the term “about.” Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±15% of the stated value; ±10% of the stated value; ±5% of the stated value; ±4% of the stated value; ±3% of the stated value; ±2% of the stated value; ±1% of the stated value; or ±any percentage between 1% and 20% of the stated value.

The terms “a,” “an,” “the” and similar referents used in the context of describing the claimed subject matter (especially in the context of the following claims) are to be construed to cover both the singular and the plural, unless otherwise indicated herein or clearly contradicted by context.

The following exemplary embodiments and examples illustrate exemplary methods provided herein. These exemplary embodiments and examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### Exemplary Embodiments

Exemplary Embodiments include but are not limited to:

1. The methods described herein includes the use of nucleic acid obtained from a biological sample of a subject for the diagnosis, detection and/or prediction of CHD. The subject can be a fetus, embryo, newborn, infant, child, adolescent, or an adult. The subject can be a pregnant woman. The biological sample include tissue sample including placental tissue or body fluid such as blood, plasma, serum, urine, saliva, sputum, sweat, tears, genital secretion including cervical secretion, amniotic fluid, and umbilical cord blood obtained at birth. In embodiments, the nucleic acid is DNA. The DNA can be cellular DNA or cell-free (cf) DNA. The cf DNA can be cf fetal (cfF) DNA.
2. The methods described herein includes the use of DNA and cfF DNA to determine the epigenetic mechanism of CHD.
3. The epigenetic mechanism described herein includes all forms of epigenetic testing based on the use of DNA and cfF DNA such as DNA methylation changes and histone modification including but not limited to methylation, acetylation, sumolyation and phosphorylation. Histones are the proteins around which the DNA strands are wrapped. Histone modification helps direct changes in DNA cytosine methylation that has been discussed, and plays a pivotal role in gene
4. The nucleic acid methylation changes described herein including DNA methylation changes include the various forms of methylation changes including cytosine methylation, cytosine hydroxymethylation, and other forms of cytosine epigenetic modification. Adenine nucleotide can also undergo DNA methylation changes. Therefore, the methods described herein include the use of cfF DNA to measure cytosine modification in all its forms that would fall within the definition of adenine methylation used for the purpose of predicting or monitoring CHD using cfF DNA.
6. DNA epigenetic changes as described herein (including methylation changes in all its forms in cytosine or adenine and including histone epigenetic modification in all its forms) based on cfF DNA for the detection of other fetal congenital anomalies in which DNA methylation or histone modification plays a role in its development, for example, neural tube defects, cleft lip, and palate.
7. The methods described herein includes the use of cfF DNA for epigenetic monitoring of the effect of exposures on a pregnancy with respect to the risk of development of relevant cardiac anomalies described herein, such as alcohol, or medications, chemicals or other known risk exposures, or disorders such as maternal diabetes or hypertension in an ongoing pregnancy.
8. A method of predicting or diagnosing congenital heart defect (CHD) in a subject in need thereof, wherein the method includes assaying a biological sample, obtained from the subject, including cf nucleic acids to determine frequency or percentage of cytosine methylation at one or more loci throughout genome; and comparing the cytosine methylation level of the sample to cytosine methylation of a control sample.
9. The method of any one of embodiments 1-8, wherein the method further includes using artificial intelligence (AI) techniques.
10. The method of any one of embodiments 1-9, wherein the method further includes using (AI) techniques comprising one or more of the following machine learning algorithms: Random Forest (RF), Support Vector Machine (SVM), Linear Discriminant Analysis (LDA), Prediction of Analysis for Microarrays (PAM), Generalized Linear Model (GLM), or deep learning (DL).
11. The method of any one of embodiments 1-10, wherein the method further includes calculating the subject's risk of developing CHD.
12. The method of any one of embodiments 1-11, wherein the control sample includes one or more biological samples from one or more normal (healthy) patients or from one or more patients diagnosed with CHD.
13. The method of any one of embodiments 1-12, wherein the biological sample includes body fluid.
14. The method of any one of embodiments 1-13, wherein the biological sample includes blood, plasma, serum, urine, saliva, sputum, sweat, breath condensate, tears, genital secretion including cervical secretion, amniotic fluid, placental tissue, CVS specimen, and umbilical cord blood obtained at birth.
15. The method of any one of embodiments 1-14, wherein the cf nucleic acids include cfF nucleic acids.
16. The method of any one of embodiments 1-15, wherein the biological sample includes cfF nucleic acids from first trimester, second trimester, and/or third trimester of pregnancy.
17. The method of any one of embodiments 1-16, wherein the cf nucleic acids include DNA.
18. The method of any one of embodiments 1-17, wherein the one or more loci include one or more loci from Tables 1-6.
19. The method of any one of embodiments 1-18, wherein the one or more loci include at least two, at least three, at least four, at least five, at least six, at least seven, at least eight, at least nine, at least 10 loci from Tables 1-6.
20. The method of any one of embodiments 1-19, wherein the one or more loci include an AUC (with 95% CI) of greater than 0.70, 0.75, 0.80, 0.85, 0.90, 0.91, 0.92, 0.93, 0.94, 0.95, 0.96, or 0.97.
21. The method of any one of embodiments 8-20, wherein the assay is a bisulfite-based methylation assay or a whole-genome methylation assay.
22. The method of any one of embodiments 1-21, wherein the one or more loci include cg06301252, cg02807450, or cg12900404.
23. The method of any one of embodiments 1-22, wherein the one or more loci include cg04761177, cg21431091, cg01263077, cg09853933, cg27142059, cg16551159, cg14957943, cg06978680, or cg12592721.
24. The method of any one of embodiments 1-23, wherein the method further includes treating the CHD.
25. The method of any one of embodiments 1-24, wherein the method further includes treating the subject by administering medication and/or performing surgery on the subject.
26. The method of any one of embodiments 1-23, wherein the method prevents CHD in future pregnancy.
27. The method of embodiment 26, wherein the method includes supplementing the pregnant mother with folic acid or folates.

### EXAMPLES

In this study, a total of 12 cases of CHD were analyzed. The 12 cases included the following: 1 case—Pulmonary artery atresia with Pulmonary valve stenosis, 4 cases—Ventricular septal defects (aka VSD); 1-case of Truncus arteriosus; 2-cases of Tetralogy of Fallot; 1-case of pulmonary artery valve stenosis; 1-case of Ventricular septal defect with atrial septal defect also; 1-case of Double aortic arch and 1-case of Bicuspid A-V valve with dilated main pulmonary artery. In total there were 12 cases of CHD. The demographics are provided in Supplemental Table 51. Table 1 provides the epigenomic data. Tables 2-6 provide epigenomic data obtained in combination with artificial intelligence techniques.

Introduction. In a series of studies (Radhakrishna et al. 2018; Bahado-Singh et al. 2019a; Radhakrishna et al. 2019), methylation changes were demonstrated in the placental DNA identified gene and gene networks that were epigenetically dysregulated in isolated VSD and non-syndromic tetralogy of Fallot, two of the most important categories of CHD. The studies helped shed important light on the pathogenic mechanisms of these CHDs. Further, CHD was accurately screened using DNA methylation markers from the placenta. While the placenta, given its abundance and limited clinical value, is ideal for such studies, applying the results to the prenatal prediction of CHD represents a significant challenge. Obtaining placental trophoblast tissue useful for DNA analysis generally requires invasive procedures such as chorionic villus sampling or placental biopsy (Alfirevic et al. 2003). The procedure is painful, requires specialist expertise, and is potentially associated with increased risk for pregnancy complications. These prior studies did not however address the issue of whether cf DNA released from the placenta into the circulation could be used to detect CHD in the developing fetus or embryo.

In the last two decades, genomic analysis of cfF DNA, present in maternal circulation in pregnancy has now progressed to the wide clinical utilization for the detection of fetal aneuploidies (Goldwaser & Klugman 2018) and other chromosomal (Ke et al. 2015; Grace et al. 2016) and molecular pathologies (Stewart et al. 2018). “Cell-free fetal DNA” however, is an inexact term as the DNA is actually from the placenta itself, an embryological fetal tissue. There is constant proliferation, differentiation, and apoptosis of the placental trophoblast (Taglauer et al. 2014). Placental apoptotic material is continuously shed into the maternal circulation. This trophoblast apoptotic material constitutes the cell-free “fetal” DNA found in the maternal blood (Gupta et al. 2004; Tjoa et al. 2006) and clinical studies (Wataganara et al. 2005; Alberry et al. 2007). Cell-free “fetal” DNA constitutes a significant percentage of the cf DNA in the maternal circulation. The contribution of the fetal friction to overall maternal cf DNA blood levels accelerates with advancing gestational age (Rafaeli-Yehudai et al. 2018). In the following study, cf DNA was extracted from the plasma (Zolotukhina et al. 2005) of pregnant women. DNA methylation analysis was performed on the cf DNA. DNA methylation analysis based on cf DNA was used to help elucidate the epigenetic pathogenesis of CHD development and also to predict non-syndromic CHD based on the DNA methylation changes observed in the circulating cfF DNA in the maternal circulation of mid-trimester pregnancies.

Methods: Study samples and cf DNA extraction. The human ethical committee has approved the present study and each subject of the study has provided informed written consent. A total of 12 cases and 26 controls were analyzed. The subjects were the mothers who gave birth to CHD cases and normal babies. The mean age of cases was 29.7 and controls were 31.3 years. cfFDNA was extracted from maternal blood. Blood samples were obtained from the subjects during pregnancy. The blood samples were drawn directly into Streck Cell-Free DNA BCT® tubes, so as to ensure the good quality of cf-DNA from the plasma (Bartak et al. 2019). Following this, the sampled tubes were processed further by centrifuging them at 3000×g for 15 minutes within the 24 hours of blood draw. The plasma was aliquoted into 2.0 ml micro-centrifuge collection tubes without disturbing buffy coat of the sample. The aliquoted plasma samples were stored at −80° C. until further process (Sheinerman et al. 2017). Five ml of plasma was used to extract cf-DNA using QIAamp circulating nucleic acid kit (Qiagen Cat #55114). The process used was a manual vacuum process using QIAvac 24 Plus vacuum manifold following the manufacturer's protocol. The silica membrane technology which can bind fragmented DNA enabled the efficient recovery of DNA. The method allowed us to use the DNA for further bisulfite conversion and methylation profiling by maintaining the methylation status of DNA.

Bisulfite conversion and methylation array processing. The bisulfite conversion of DNA was performed using EZ DNA Methylation Gold Kit (Zymo, USA) according to the company's protocol using 10 ul of elution buffer (Hardy et al. 2017). The methylation profiling was performed using Illumina Infinium Methylation EPIC BeadChip arrays with over 850K methylation markers according to the manufacturer's instructions. Samples were randomized on the chips. Both cases and control sample process were performed together to avoid any sample processing bias. The arrays were dried using a vacuum drier and the processed BeadChips were imaged using illumina iScan System as per the company's instructions.

Statistical and bioinformatic analysis. After scanning, the raw iDat files were downloaded from the iScan software and the data were analysed using GenomeStudio software. The β-values of cases versus control subjects were compared for individual CpG loci to perform differential methylation analysis. Previous publications described further downstream statistical analysis (Bahado-Singh et al. 2019a; Bahado-Singh et al. 2019b). For the further downstream analysis, CpG probes constituting X and Y chromosomes followed by dbSNP entries within 10 bp of CpG sites were excluded to avoid gender bias and the genetic effects respectively on methylation sites (Wilhelm-Benartzi et al. 2013). The β-value with BH adjusted FDR p-value <0.01 was considered as cutoff for the significance. The Area Under the Receiver Operating Characteristic (AUC-ROC) curves with 95% CI was calculated for each significant CpG loci using dplyr, reshape2 and ROCR packages of R tool.

Artificial Intelligence (AI) Analysis. The methods used for the AI analyses performed are described in Bahado-Singh et al. 2019b. What follows is a summary of the previously published descriptions. The epigenomic data were divided into two groups, a training group consisting of 80% of the study subjects and a test group that constituted the remaining 20%. It is an approach that is frequently used when analyzing smaller small data sets. We performed 10-fold cross-validation on the data from the training group in order to generate the prediction model. This model was then appraised in the independent validation or test group. Six appropriate AI approaches or algorithms were used to determine screening performance for CHD detection. These were Random Forest (RF), Support Vector Machine (SVM), Linear Discriminant Analysis (LDA), Prediction Analysis for Microarrays (PAM), Generalized Linear Model (GLM), and Deep Learning (DL) which is the newest form of AI and which is increasingly being used in the analysis of complex and voluminous biological data such as epigenomics.

RF is a supervised classification algorithm for classification, regression, and other functions. A forest of decision trees is randomly created and the mean prediction of the individual trees is determined. There is a direct correlation between the number of trees in the forest and the accuracy of the results that are generated. Increasing the number of trees will increase the accuracy of the results that are obtained. RF has the benefits of being able to work with missing values in a data-set and can utilize categorical values (Huang et al. 2013). SVM is first fed with labelled data (supervised learning) identifying the different groups and from this builds a model for distinguishing the groups. Subsequently, when provided with unlabelled fresh data it is able to develop models or hyperplanes to cluster one group from another. SVM was able to perform both regression and classification tasks and can handle multiple continuous and categorical variables (Mahadevan et al. 2008). LDA was used to reduce the number of features or predictors need to accurately classify and discriminate the groups. This is particularly useful for epigenomic analysis as the study started out with close to 900,000 potential features to be used for CHD detection. LDA is simple in its approach but can still achieve excellent accuracy. Accuracy is as good as more complex methods. LDA is based on the identification of a linear combination of variables (predictors) that best separates the two classes (targets) (Liland 2011). It is closely related to the analysis of variance (ANOVA) and regression analysis which attempts to define an outcome variable based on a combination of explanatory variables. PAM is a statistical technique for class prediction from gene expression data using the nearest shrunken centroids (Alakwaa et al. 2018; Candel et al. 2018). This method identifies the subsets of genes that best characterize each class. GLMs are a broad class of models that include linear regression, ANOVA, Poisson regression, log-linear models, and others (Alakwaa et al. 2018; Candel et al. 2018). DL is a form of representation learning that uses multiple transformation steps to create very complex features. DL is categorized into feed-forward artificial neural networks (ANNs), which uses more than one hidden layer (y) that connects the input (x) and output layer (z) via a weight O) matrix. The weight matrix is expected to minimize the difference between the input and output layers and is considered as the best AI approach (Alakwaa et al. 2018; Candel et al. 2018).

Multivariate Regression Analysis provided with model. Before the multivariate regression analysis was performed epigenomics data were subjected to quantile normalization and auto-scaling. As a quality control step, in an attempt to investigate the existence of any systematic variation and to detect potential outlier(s), principal component analysis (PCA) was performed on all classes using MetaboAnalyst (v4.0) (Chong et al. 2018). Subsequently, these pre-processed data were used to perform partial least-squares discriminant analysis (PLSDA). The ideal number of CpG β-value variables were carefully chosen based on predictive accuracy and cross validation using leave one out cross-validation method available in MetaboAnalyst. Goodness of fit (R2) and predictability (Q2) values for each PLSDA model have been reported. On cross-validation, a 2000 iteration permutation test was performed that can minimize the possibility of observed separation on PLSDA was due to chance considering the p-value <0.05.

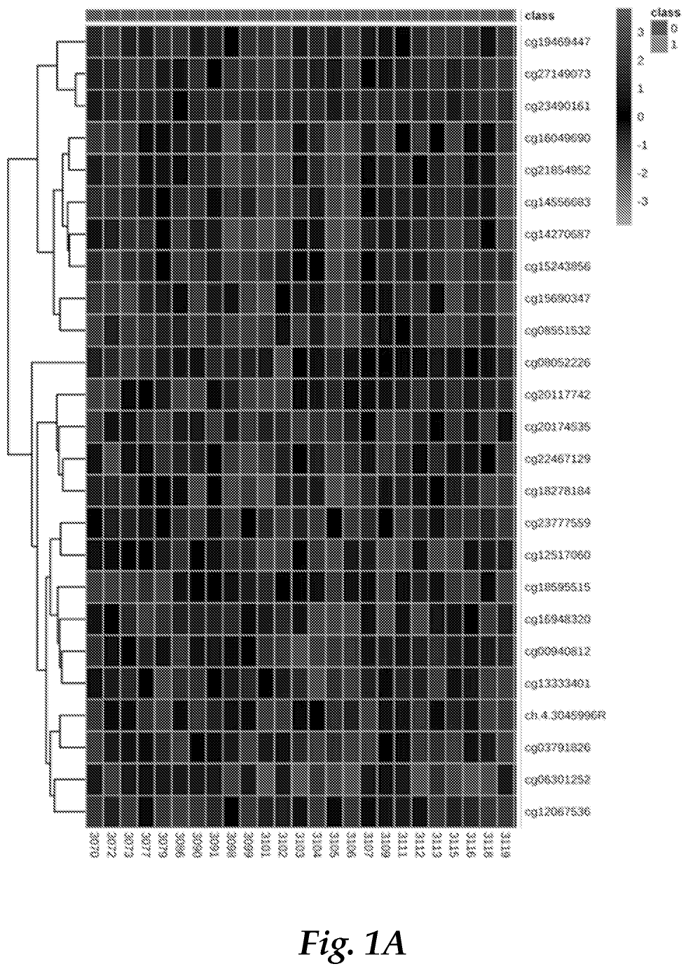

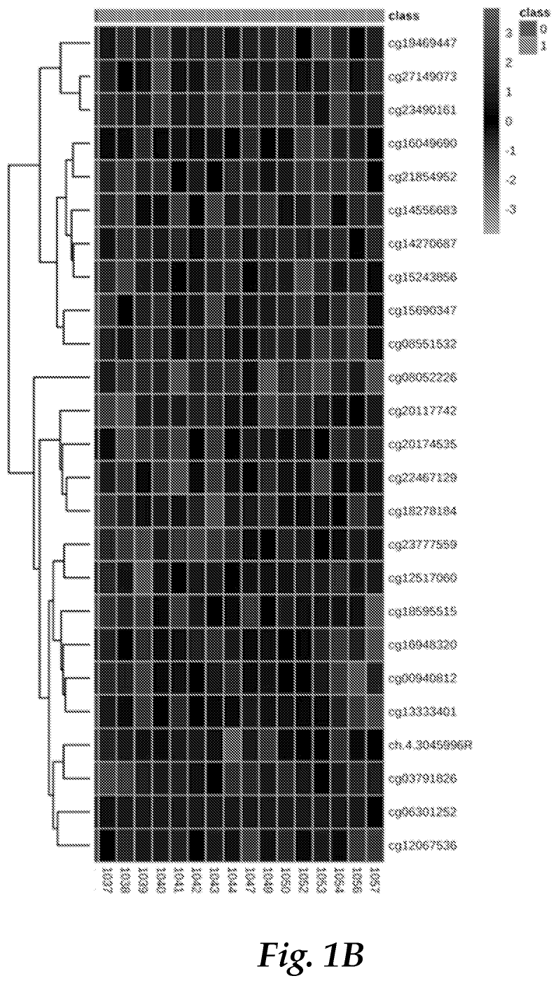

Cluster analysis of differentially methylated targets in cfF DNA CHD. A heatmap was generated using individual β-values for the significantly differentially methylated markers between cases and controls (FIG. 1). Missing values were imputed with median values of a particular methylation site if there is less than 20% of missing values exist. Any methylated markers showing greater than 20% of missing values were omitted. Further, data were filtered using interquantile range (IQR) and quantile normalized to avoid dilution effect. On hierarchical cluster analysis, Euclidean distance measure was used along with ward clustering algorithm and viewed with PLADA-VIP plot scores. The control and case groups were given codes, 0 and 1 respectively.

Gene ontology analysis and functional enrichment. The genes found to be significantly differentially methylated with FDR p-value <0.01 were used to perform disease and functional enrichment analysis using Ingenuity Pathway Analysis (IPA) (Qiagen IPA) system. The IPA platform enables systemic analysis of array data associated with the biological function (Haddad et al. 2016). The gene networks were considered based on their inter-relationship and role in cardiac development and diseases.

Results. Genome-wide methylation profiling was performed using cfF DNA extracted from the mothers who gave birth to 12 CHD cases and 26 controls. The gestational age at the time of sampling also did not show any statistical difference (p-value=0.15). The mean (SD) gestational age at blood draw was 23 weeks 5 days for cases and 24 weeks 6 days for controls (p−0.15). The age range and gestational age at the time of sampling of mothers who gave birth to CHD babies and mothers who gave birth to normal babies did not show significant difference with the p-value of 0.32 and 0.15 respectively. The details of the demographics are provided in Table 51.

TABLE S1
Clinical and Demographic Characteristics of Study Subjects*

| | Mean (SD) | | Mean (SD) | | p- |
|---|---|---|---|---|---|
| | of Controls | | of CHD | | value |
| Number of subjects | 12 | | 26 | | — |
| Age (years) | 31.36 | (4.61) | 29.73 | (5.59) | 0.32 |
| Gravidity | 2.36 | (0.75) | 2.40 | (0.82) | 0.79 (W) |
| Parity | 2.60 | (1.08) | 1.86 | (1.06) | 0.03 (W) |
| GA during sampling | 166.12 | (38.87) | 173.93 | (38.05) | 0.15 (W) |
| (days) | | | | | |
| BMI | 30.93 | (7.30) | 23.31 | (10.01) | 0.03 (W) |
| Number with prior | 1.12 | (0.33) | 1.00 | (0.00) | 0.08 |
| child with CHD | | | | | |
| Family history of CHD | 1.24 | (0.43) | 1.13 | (0.35) | 0.43 (W) |
| 1st trimester alcohol | 1.20 | (0.40) | 1.20 | (0.41) | 1.00 (W) |
| use | | | | | |
| 1st trimester tobacco | 1.04 | (0.20) | 1.13 | (0.35) | 0.29 (W) |
| use | | | | | |

W: Wilcoxon Rank Sum Statistical test
*There was no difference in racial composition of the two groups.

Differential methylation analysis of cfF DNA identified a total of 5918 CpG markers encompassing 4976 genes (FDR p-value ≤01). The top 1000 significant CpG loci and associated genes with University of California Santa Cruz (UCSC) gene symbol, and the statistics is been provided in the Table 1. Of these markers 130 CpGs were hypomethylated and 870 markers were hypermethylated in association with CHD. 53 hypomethylated and 486 hypermethylated markers were found to be differentially methylated by ≥10% methylation difference. The 3 markers those were above 20% of methylation difference were, cg06301252 (PTPRN2) with 33.62%, cg02807450 (MTMR2) with −21.15% methylation change and cg12900404 (DOCK10) with −20.13%. 126 CpG loci showed AUC ≥0.80 individually, indicating the excellent predictive accuracy for the disease prediction.

AI prediction of methylation markers for cfF DNA CHD. The significantly differentially methylated CpGs with 5% of difference and AUC >0.70 has been used to perform AI analysis using 6 predictive algorithms. Among them, RF and SVM model was found to show highest performance with AUC (95% CI)=0.98 (0.831−1) with 93.8% sensitivity and 93.2% specificity and with AUC (95% CI)=0.97 (0.877−1) with 98% sensitivity and 94% specificity, respectively, followed by DL model with AUC (95% CI)=0.94 (0.840−1.0) with 93% sensitivity and 94% specificity (Table 2). High performance was also achieved for each of the other four platforms. Comparable predictive performance was achieved when demographic markers were considered along with CpG loci (Table 3) and also high performance was achieved when only markers meeting stringent GWAS thresholds were considered (Table 4). High performance was achieved when demographics markers and CpG loci meeting stringent GWAS thresholds were considered (Table 5). Table 6 shows that when only markers showing high level of methylation change, for example 1.5 fold or greater, are used high predictive accuracies are seen. The top 5 predictive markers for each model are provided in each table in a descending order (under each table).

Logistic Regression analysis. Conventional logistic regression analysis with 10-fold cross validation was performed and compared markers with the AI prediction for the CpGs with 5% of difference and AUC >0.70. On training set, AUC (95% CI) of 0.98 (0.98−0.97) with 100% sensitivity and 85% specificity was obtained, following the training logistic regression model with a 10-fold cross validation provided AUC values (95% CI) of 0.79 (0.61−0.98) with 83% sensitivity and 80% specificity provided on a test set. The logistic regression equation was as follows,

logit(P)=log (P/(1−P))=−5.469−2.698 cg08230215−3.924 cg04761177−2.478 cg10259004−2.477 cg06009031.

For the CpG markers with stringent p-value 5×10⁻⁸, AUC (95% CI)=0.99 (0.92−0.99) with 90% sensitivity and 95% specificity on training/discovery test was found. For the 10-fold cross validation showed AUC (95% CI)=0.71 (0.50−0.92) with 75% sensitivity and 87% specificity having the equation:

logit(P)=log(P/(1−P))=−2.583−5.115cg04761177−6.351cg23306063+1.126cg10259004−0.369cg23136742−2.917cg11196182.

Cluster analysis of differentially methylated targets in cfF DNA CHD. In hierarchical cluster analysis using individual β-values for significantly differentially methylated markers between cases and controls, well separated clusters for hypo and hypermethylation among cases and controls were observed (FIGS. 1A and 1B). The CpG marker IDs are provided in the right column.

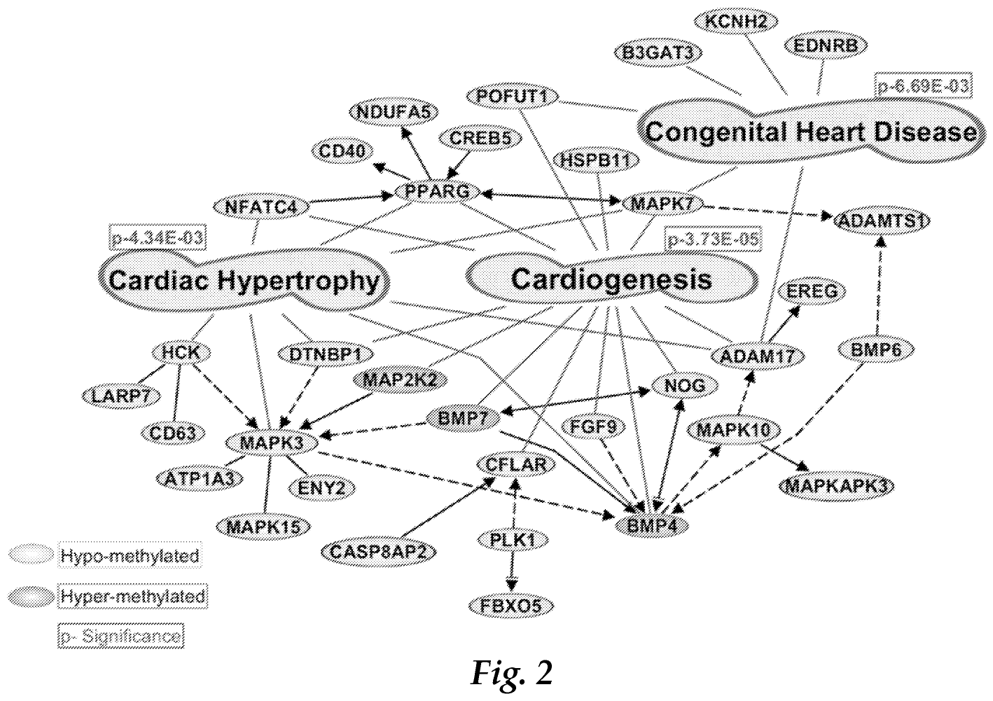

Disease and functional enrichment analysis. To determine whether the statistical findings had biological plausibility, pathway analyses were performed. Ingenuity Pathway systems showed significant disease and functional enrichment of the genes associated with CHD. The top 3 cardiac development and disease function showed enrichment in Congenital Heart Disease (p-6.69E-03), Cardiac Hypertrophy (p-4.34E-03) and Cardiogenesis (p-3.73E-05). FIG. 2 depicts the network of genes involved among these disease functions. Thus, important genes that were differentially methylated appeared to play a role in cardiac embryology and CHD development.

Discussion A comprehensive methylation profiling combined with AI prediction were performed using the free circulating cfF DNA from mother's blood. The study indicated the significant differential methylation of 5918 CpG markers comprising 4976 genes between cases and controls. cf-DNA is one of the promising biomarker source for prenatal diagnosis which is minimally invasive and slowly substituting the other invasive tests such as amniocentesis or chorionic villus sample based tests (Nagy 2019). Epigenetic factors regulate gene expression and affects heart development during embryogenesis (Vallaster et al. 2012). The 1278 hypomethylated genes tend to overexpress in the cells (Klasic et al. 2016) while the remaining 4640 hypermethylated genes may show downregulated expression in the cells (Razin & Kantor 2005). 539 CpGs showed methylation difference 10% indicating the biological relevance of gene expression. The study samples were found to be separated based on the PLS-DA analysis and the differences between hyper and hypomethylated markers are shown in the heatmap (FIG. 1).

AI analysis was performed using 6 different algorithms including SVM, GLM, PAM, RF, LDA and DL. The SVM model provided the best prediction with an AUC=0.97 (98% sensitivity and 94% specificity). The top 5 predictive markers include cg04761177 (ATP2A1), cg21431091 (TMEM9), cg01263077 (MYO9B), cg09853933 (ATG2B; GSKIP) and cg27142059 (TRIM15). To compare with the AI prediction, the markers were tested using conventional logistic regression analysis with 10 fold cross validation and found cg08230215 (MAST3), cg04761177 (ATP2A1), cg10259004 (MYL9) and cg06009031 (C7orf50). The common marker among predictive algorithms SVM, PAM, RF, LDA, DL and logistic regression was found to be cg04761177 (ATP2A1). cg04761177 has been hypomethylated with 9% methylation difference, with AUC=0.94 (CI 0.86-1.00) for CHD prediction and the gene ATP2A1 is also termed as SERCA1. This gene regulates the electrical and contractile properties of heart and thereby dysregulation is associated with diverse heart diseases including Congestive Heart Failure (Peters et al. 1997; Ennis et al. 2002).

The second common CpG marker predicted to be excellent marker from both AI algorithms with stringent p-value-5×10-8 and conventional logistic regression was cg10259004 (MYL9). MYL9 gene codes for Myosin Light Chain, and expresses in the cardiac smooth muscles and participate in the morphogenesis of heart (England & Loughna 2013).

Disease and functional enrichment of genes with CpGs in cf DNA associated with CHD. The disease based pathway enrichment analysis has identified significantly differentially methylated genes that are currently known or predicted to be associated with cardiac hypertrophy, cardiogenesis and congenital heart disease. This indicates the biological plausibility of the identified genes in association with CHD. All identified CpG methylation sites were identified within promoter or in the gene region and associated with mechanism of cardiac hypertrophy, cardiogenesis and congenital heart disease with a significant p-value <0.001. For example, some of the hypomethylated genes include, HSPB11, POFUT1, NFATC4, DTNBP1, CFLAR, KCNH2, B3GAT3, BMP4 and the hypermethylated genes include, BMP7, NOG, MAP2K2, FGF9, ADAM17 and MAPK3.

HSPB11 was found to be associated with cardiogenesis and highly expressed in later stages of ventricular tissue development in zebrafish (Singh et al. 2016). POFUT1, is an essential component of Notch signalling, that plays a vital role in the development of the heart valves, cardiac outflow tracts and ventricular septum formation (Penton et al. 2012). The mouse deficient with protein POFUT1 die during the mid-gestation with severe defects in vasculogenesis and cardiogenesis (Shi & Stanley 2003). NFATC4 and DTNBP1 were involved in inducing human cardiac hypertrophy (Poirier et al. 2003; Rangrez et al. 2013). Reduced expression of CFLAR gene have shown to exhibit severe defects on cardiac trabecular formation, thinner myocardium, cardiac lethality and cardiac ventricular structures (Imanishi et al. 2000; Yeh et al. 2000; Lakhani et al. 2006; Ye et al. 2013). KCNH2, codes for a protein known as Kv11.1 (potassium ion channel) which conducts potassium ions out of the cardiac myocytes (Newton-Cheh et al. 2007; Park et al. 2013). A retrospective study in large cohort investigated that mutations in KCNH2 results in atrioventricular block, tetralogy of Fallot, Coarctation, atrial septal defect, atrioventricular canal, bicuspid aortic valve, patent ductal arteriosus, tricuspid atresia and ventricular septal defect (Ebrahim et al. 2017). Another gene, B3GAT3 plays a significant role in proteoglycan biosynthesis (von Oettingen et al. 2014) and mutation in these proteoglycan chain results in severe congenital heart anomalies including bicuspid aortic valve, ventricular septal defects, and mitral valve prolapse (Baasanjav et al. 2011; Bloor et al. 2017). BMP4 is an essential source for the development of endocardial cushion and for normal partitioning of the Outflow tract. In mice, loss of BMP4 functioning results in ventricular septal defects, abnormal semilunar valve formation, abnormal cushion remodelling, persistent truncus arteriosus and inadequate cardiac differentiation in the developing epicardial cushion (McCulley et al. 2008).

The hypermethylated genes such as BMP7 encodes for a secreted ligand of the TGF-beta that plays a key regulatory role in coronary vasculature, ventricular myocardial development and compaction (Azhar et al. 2003). In mouse embryo, low BMP7 is linked to increased cardiovascular disease morbidity and mortality (Silverman et al. 2004) (Freedman et al. 2009). Another hypermethylated gene, NOG (Noggin) has strong interaction with BMP2 and BMP4, however it also interacts with BMP7 (Choi et al. 2007). It is notable that both BMP4 and BMP7 has been differentially methylated in the present study along with NOG. The Noggin knocked-out mice shows several anomalies of cardiovascular development those are the results of BMP signaling and predicted to play analogous mechanism in humans (Choi et al. 2007). MAP2K2 (Mitogen-Activated Protein Kinase Kinase 2), another hypermethylated gene, earlier reported to be associated with increased prevalence of Cardiac hypertrophy by activating ERK pathway (Gillespie-Brown et al. 1995; Gallo et al. 2019). The other map kinase family gene, MAPK3 is involved in the regulation of meiosis, mitosis and post-mitotic function and the dysregulation in this gene advocates cardiac hypertrophy (Bueno et al. 2000; Mutlak & Kehat 2015). FGF9 gene is largely expressed in the epicardium that maintains myocardial proliferation during mid-gestational cardiac development. Altered expression of FGF9 is associated with significantly decreased cardiomyoblast proliferation and ventricular hypoplasia. (Lavine et al. 2005). FGF9 also functions as paracrine signals in the embryonic heart development and loss of function is associated with decreased cardiomyocyte proliferation (Itoh et al. 2016). A Disintegrin And Metalloproteinase Domain-Containing Protein 17 gene (ADAM17) plays a significant role in structural cardiac remodeling by changing cell-surface matrix receptors and the loss of function of ADAM17 contributes to cardiac hypertrophy (Wang et al. 2009; Takayanagi et al. 2016). Altered expression of ADAM17 has also been marked with ventricular remodelling suggesting its important role in late stages of cardiac remodelling (Zheng et al. 2016).

This proof of concept study confirms the effect of DNA methylation on causing CHD and identifying this using cfF DNA is a possible approach. The diagnostic accuracies predicted using various statistical methods and AI platform helped to prioritize the genes those are novel for CHD prediction.

In conclusion, significantly differentially methylated markers associated with CHD in cfF DNA were identified. The AI algorithms predicted significant markers associated with CHD and the disease enrichment analysis showed important genes associated with cardiac development and function. Developing minimally invasive methods to perform prenatal diagnosis is of clinical importance. Understanding the role and regulation of the identified genes using cfF DNA in this study would further form the basis of understanding the molecular mechanisms for the embryological development of the normal and abnormal heart of the earliest stages of pregnancy.

The subject matter described above is provided by way of illustration only and should not be construed as limiting. Various modifications and changes may be made to the subject matter described herein without following the example embodiments and applications illustrated and described, and without departing from the true spirit and scope of the present disclosure, which is set forth in the following claims.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

### REFERENCES

Alakwaa F. M., Chaudhary K. & Garmire L. X. (2018) Deep Learning Accurately Predicts Estrogen Receptor Status in Breast Cancer Metabolomics Data. J Proteome Res 17, 337-47.
Alberry M., Maddocks D., Jones M., Abdel Hadi M., Abdel-Fattah S., Avent N. & Soothill P. W. (2007) Free fetal DNA in maternal plasma in anembryonic pregnancies: confirmation that the origin is the trophoblast. Prenat Diagn 27, 415-8.
Alfirevic Z., Sundberg K. & Brigham S. (2003) Amniocentesis and chorionic villus sampling for prenatal diagnosis. Cochrane Database Syst Rev, CD003252.
Azhar M., Schultz Jel J., Grupp I., Dorn G. W., 2nd, Meneton P., Molin D. G., Gittenberger-de Groot A. C. & Doetschman T. (2003) Transforming growth factor beta in cardiovascular development and function. Cytokine Growth Factor Rev 14, 391-407.
Baasanjav S., Al-Gazali L., Hashiguchi T., Mizumoto S., Fischer B., Horn D., Seelow D., Ali B. R., Aziz S. A., Langer R., Saleh A. A., Becker C., Nurnberg G., Cantagrel V., Gleeson J. G., Gomez D., Michel J. B., Stricker S., Lindner T. H., Nurnberg P., Sugahara K., Mundlos S. & Hoffmann K. (2011) Faulty initiation of proteoglycan synthesis causes cardiac and joint defects. Am J Hum Genet 89, 15-27.
Bahado-Singh R., Vishweswaraiah S., Mishra N. K., Guda C. & Radhakrishna U. (2019a) Placental DNA methylation changes for the detection of tetralogy of Fallot. Ultrasound Obstet Gynecol.
Bahado-Singh R. O., Vishweswaraiah S., Aydas B., Mishra N. K., Guda C. & Radhakrishna U. (2019b) Deep Learning/Artificial Intelligence and Blood-Based DNA Epigenomic Prediction of Cerebral Palsy. Int J Mol Sci 20.
Bahado-Singh R. O., Wapner R., Thom E., Zachary J., Platt L., Mahoney M. J., Johnson A., Silver R. K., Pergament E., Filkins K., Hogge W. A., Wilson R. D., Jackson L. G., First Trimester Maternal Serum B. & Fetal Nuchal Translucency Screening Study G. (2005) Elevated first-trimester nuchal translucency increases the risk of congenital heart defects. Am J Obstet Gynecol 192, 1357-61.
Bahado-Singh R. O., Zaffra R., Albayarak S., Chelliah A., Bolinjkar R., Turkoglu O. & Radhakrishna U. (2016) Epigenetic markers for newborn congenital heart defect (CHD). J Matern Fetal Neonatal Med 29, 1881-7.
Bartak B. K., Kalmar A., Galamb O., Wichmann B., Nagy Z. B., Tulassay Z., Dank M., lgaz P. & Molnar B. (2019) Blood Collection and Cell-Free DNA Isolation Methods Influence the Sensitivity of Liquid Biopsy Analysis for Colorectal Cancer Detection. Pathol Oncol Res 25, 915-23.
Bloor S., Girl D., Didi M. & Senniappan S. (2017) Novel Splicing Mutation in B3GAT3 Associated with Short Stature, GH Deficiency, Hypoglycaemia, Developmental Delay, and Multiple Congenital Anomalies. Case Rep Genet 2017, 3941483.
Botto L D, Mulinare J, Erickson J D. Do multivitamin or folic acid supplements reduce the risk for congenital heart defects? Evidence and gaps. Am J Med Genet A 2003; 121A:95-101)
Botto L D, Yang Q. 5,10-Methylenetetrahydrofolate reductase gene variants and congenital anomalies: a HuGE review. Am J Epidemiol 2000; 151:862-77
Bueno O. F., De Windt L. J., Tymitz K. M., Witt S. A., Kimball T. R., Klevitsky R., Hewett T. E., Jones S. P., Lefer D. J., Peng C. F., Kitsis R. N. & Molkentin J. D. (2000) The MEK1-ERK1/2 signaling pathway promotes compensated cardiac hypertrophy in transgenic mice. EMBO J 19, 6341-50.
Burton G. J. & Jauniaux E. (2018) Development of the Human Placenta and Fetal Heart: Synergic or Independent? Front Physiol 9, 373.
Candel A., Parmar V., LeDell E. & Arora A. (2018) Deep Learning with H2O.
Choi M., Stottmann R. W., Yang Y. P., Meyers E. N. & Klingensmith J. (2007) The bone morphogenetic protein antagonist noggin regulates mammalian cardiac morphogenesis. Circ Res 100, 220-8.
Chong J., Soufan O., Li C., Caraus I., Li S., Bourque G., Wishart D. S. & Xia J. (2018) MetaboAnalyst 4.0: towards more transparent and integrative metabolomics analysis. Nucleic Acids Res 46, W486-W94.
Czeizel A E, Merhala Z. Bread fortification with folic acid, vitamin B12, and vitamin B6 in Hungary. Lancet 1998; 352:1225
Ebrahim M. A., Williams M. R., Shepard S. & Perry J. C. (2017) Genotype Positive Long QT Syndrome in Patients With Coexisting Congenital Heart Disease. Am J Cardiol 120, 256-61.
England J. & Loughna S. (2013) Heavy and light roles: myosin in the morphogenesis of the heart. Cell Mol Life Sci 70, 1221-39.
Ennis I. L., Li R. A., Murphy A. M., Marban E. & Nuss H. B. (2002) Dual gene therapy with SERCA1 and Kir2.1 abbreviates excitation without suppressing contractility. J Clin Invest 109, 393-400.
Freedman B. I., Bowden D. W., Ziegler J. T., Langefeld C. D., Lehtinen A. B., Rudock M. E., Lenchik L., Hruska K. A., Register T. C. & Carr J. J. (2009) Bone morphogenetic protein 7 (BMP7) gene polymorphisms are associated with inverse relationships between vascular calcification and BMD: the Diabetes Heart Study. J Bone Miner Res 24, 1719-27.
Gallo S., Vitacolonna A., Bonzano A., Comoglio P. & Crepaldi T. (2019) ERK: A Key Player in the Pathophysiology of Cardiac Hypertrophy. Int J Mol Sci 20.
Gillespie-Brown J., Fuller S. J., Bogoyevitch M. A., Cowley S. & Sugden P. H. (1995) The mitogen-activated protein kinase kinase MEK1 stimulates a pattern of gene expression typical of the hypertrophic phenotype in rat ventricular cardiomyocytes. J Biol Chem 270, 28092-6.
Goldwaser T. & Klugman S. (2018) Cell-free DNA for the detection of fetal aneuploidy. Fertil Steril 109, 195-200.
Grace M. R., Hardisty E., Dotters-Katz S. K., Vora N. L. & Kuller J. A. (2016) Cell-Free DNA Screening: Complexities and Challenges of Clinical Implementation. Obstet Gynecol Surv 71, 477-87.
Gupta A. K., Holzgreve W., Huppertz B., Malek A., Schneider H. & Hahn S. (2004) Detection of fetal DNA and RNA in placenta-derived syncytiotrophoblast microparticles generated in vitro. Clin Chem 50, 2187-90.
Haddad D., Socci N., Chen C. H., Chen N. G., Zhang Q., Carpenter S. G., Mittra A., Szalay A. A. & Fong Y. (2016) Molecular network, pathway, and functional analysis of time-dependent gene changes associated with pancreatic cancer susceptibility to oncolytic vaccinia virotherapy. Mol Ther Oncolytics 3, 16008.
Huang J. H., Xie H. L., Yan J., Lu H. M., Xu Q. S. & Liang Y. Z. (2013) Using random forest to classify T-cell epitopes based on amino acid properties and molecular features. Anal Chim Acta 804, 70-5.
Imanishi T., Murry C. E., Reinecke H., Hano T., Nishio I., Liles W. C., Hofsta L., Kim K., O'Brien K. D., Schwartz S. M. & Han D. K. (2000) Cellular FLIP is expressed in cardiomyocytes and down-regulated in TUNEL-positive grafted cardiac tissues. Cardiovasc Res 48, 101-10.
Ionescu-Ittu R, Marelli A J, Mackie A S et al. Prevalence of severe congenital heart disease after folic acid fortification of grain products: time trend analysis in Quebec, Canada. BMJ 2009; 338:b1673 doi:10.1136/bmj.b1673
Itoh N., Ohta H., Nakayama Y. & Konishi M. (2016) Roles of FGF Signals in Heart Development, Health, and Disease. Front Cell Dev Biol 4, 110.
Karunamuni G, Sheehan M M, Doughman Y Q et al. Supplementation with the methyl donor betaine prevents congenital defects induced by prenatal alcohol exposure. Alcohol Clin Exp Res 2017; 41:1917-27
Ke W. L., Zhao W. H. & Wang X. Y. (2015) Detection of fetal cell-free DNA in maternal plasma for Down syndrome, Edward syndrome and Patau syndrome of high risk fetus. Int J Clin Exp Med 8, 9525-30.
Klasic M., Kristic J., Korac P., Horvat T., Markulin D., Vojta A., Reiding K. R., Wuhrer M., Lauc G. & Zoldos V. (2016) DNA hypomethylation upregulates expression of the MGAT3 gene in HepG2 cells and leads to changes in N-glycosylation of secreted glycoproteins. Sci Rep 6, 24363.
Kriaucionis S, Heintz N. The nuclear DNA base 5-hydroxymethylcytosine is present in Purkinje neurons and the brain. Science 2009; 324:929-30
Lakhani S. A., Masud A., Kuida K., Porter G. A., Jr., Booth C. J., Mehal W. Z., Inayat I. & Flavell R. A. (2006) Caspases 3 and 7: key mediators of mitochondrial events of apoptosis. Science 311, 847-51.
Lavine K. J., Yu K., White A. C., Zhang X., Smith C., Partanen J. & Ornitz D. M. (2005) Endocardial and epicardial derived FGF signals regulate myocardial proliferation and differentiation in vivo. Dev Cell 8, 85-95.
Lehmann-Werman R, Neiman D, Zemmour H et al. Identification of tissue-specific cell death using methylation patterns of circulating DNA. Proc Natl Acad Sci U.S.A. 2016:113(13):E1826-34
Liland K. H. (2011) Multivariate methods in metabolomics—from pre-processing to dimension reduction and statistical analysis. TrAC Trends in Analytical Chemistry 30, 827-41.
Liu Y, Balaraman Y, Wang G et al. Alcohol exposure alters DNA methylation profiles in mouse embryos at early neurulation. Epigenetics 2009; 4:500-11
Mahadevan S., Shah S. L., Marrie T. J. & Slupsky C. M. (2008) Analysis of metabolomic data using support vector machines. Anal Chem 80, 7562-70.
Mao B, Qui J, Zhao N et al. Maternal folic acid supplementation and dietary folate intake and congenital heart defects. PLoS One 2017 Nov. 16; 12:e0187996. doi: 10.1371/journal.pone.0187996
Maslen C. L. (2018) Recent Advances in Placenta-Heart Interactions. Front Physiol 9, 735.
McCulley D. J., Kang J. O., Martin J. F. & Black B. L. (2008) BMP4 is required in the anterior heart field and its derivatives for endocardial cushion remodeling, outflow tract septation, and semilunar valve development. Dev Dyn 237, 3200-9.
Mutlak M. & Kehat I. (2015) Extracellular signal-regulated kinases ½ as regulators of cardiac hypertrophy. Front Pharmacol 6, 149.
Nagy B. (2019) Cell-free nucleic acids in prenatal diagnosis and pregnancy-associated diseases. EJIFCC 30, 215-23.
Nester C E, Ottaviano R, Reddington J et al. Tissue type is a major modifier of the 5-hydroxymethylcytosine content of human genes. Genome Res 2012; 22:467-77
Newton-Cheh C., Guo C. Y., Larson M. G., Musone S. L., Surti A., Camargo A. L., Drake J. A., Benjamin E. J., Levy D., D'Agostino R. B., Sr., Hirschhorn J. N. & O'Donnell C J. (2007) Common genetic variation in KCNH2 is associated with Q T interval duration: the Framingham Heart Study. Circulation 116, 1128-36.
O'Meara C. C. & Lee R. T. (2015) Peering Into the Cardiomyocyte Nuclear Epigenetic State. Circ Res 117, 392-4.
Park J. K., Oh Y. S., Choi J. H. & Yoon S. K. (2013) Single nucleotide deletion mutation of KCNH2 gene is responsible for LQT syndrome in a 3-generation Korean family. J Korean Med Sci 28, 1388-93.
Penton A. L., Leonard L. D. & Spinner N. B. (2012) Notch signaling in human development and disease. Semin Cell Dev Biol 23, 450-7.
Peters D. G., Mitchell H. L., McCune S. A., Park S., Williams J. H. & Kandarian S. C. (1997) Skeletal muscle sarcoplasmic reticulum Ca(2+)-ATPase gene expression in congestive heart failure. Circ Res 81, 703-10.
Poirier O., Nicaud V., McDonagh T., Dargie H. J., Desnos M., Dorent R., Roizes G., Schwartz K., Tiret L., Komajda M. & Cambien F. (2003) Polymorphisms of genes of the cardiac calcineurin pathway and cardiac hypertrophy. Eur J Hum Genet 11, 659-64.
Radhakrishna U., Albayrak S., Alpay-Savasan Z., Zeb A., Turkoglu O., Sobolewski P. & Bahado-Singh R. O. (2016) Genome-Wide DNA Methylation Analysis and Epigenetic Variations Associated with Congenital Aortic Valve Stenosis (AVS). PLoS One 11, e0154010.
Radhakrishna U., Albayrak S., Zafra R., Baraa A., Vishweswaraiah S., Veerappa A. M., Mahishi D., Saiyed N., Mishra N. K., Guda C., Ali-Fehmi R. & Bahado-Singh R. O. (2019) Placental epigenetics for evaluation of fetal congenital heart defects: Ventricular Septal Defect (VSD). PLoS One 14, e0200229.
Radhakrishna U., Vishweswaraiah S., Veerappa A. M., Zafra R., Albayrak S., Sitharam P. H., Saiyed N. M., Mishra N. K., Guda C. & Bahado-Singh R. (2018) Newborn blood DNA epigenetic variations and signaling pathway genes associated with Tetralogy of Fallot (TOF). PLoS One 13, e0203893.
Rafaeli-Yehudai T., lmterat M., Douvdevani A., Tirosh D., Benshalom-Tirosh N., Mastrolia S. A., Beer-Weisel R., Klaitman V., Riff R., Greenbaum S., Alioshin A., Rodaysky Hanegbi G., Loverro G., Catalano M. R. & Erez O. (2018) Maternal total cell-free DNA in preeclampsia and fetal growth restriction: Evidence of differences in maternal response to abnormal implantation. PLoS One 13, e0200360.
Rafi I, Hill M, Hayward J et al. Non-invasive prenatal testing: use of cell-free fetal DNA in Down syndrome screening. Br J Gen Pract 2017; 67:298-299
Ramakrishnan A., Lee L. J., Mitchell L. E. & Agopian A. J. (2015) Maternal Hypertension During Pregnancy and the Risk of Congenital Heart Defects in Offspring: A Systematic Review and Meta-analysis. Pediatr Cardiol 36, 1442-51.
Rangrez A. Y., Bernt A., Poyanmehr R., Harazin V., Boomgaarden I., Kuhn C., Rohrbeck A., Frank D. & Frey N. (2013) Dysbindin is a potent inducer of RhoA-SRF-mediated cardiomyocyte hypertrophy. J Cell Biol 203, 643-56.
Razin A. & Kantor B. (2005) DNA methylation in epigenetic control of gene expression. Prog Mol Subcell Biol 38, 151-67.
Sheinerman K. S., Toledo J. B., Tsivinsky V. G., Irwin D., Grossman M., Weintraub D., Hurtig H. I., Chen-Plotkin A., Wolk D. A., McCluskey L. F., Elman L. B., Trojanowski J. Q. & Umansky S. R. (2017) Circulating brain-enriched microRNAs as novel biomarkers for detection and differentiation of neurodegenerative diseases. Alzheimers Res Ther 9, 89.
Shi S. & Stanley P. (2003) Protein 0-fucosyltransferase 1 is an essential component of Notch signaling pathways. Proc Natl Acad Sci USA 100, 5234-9.
Silverman S. L., Delmas P. D., Kulkarni P. M., Stock J. L., Wong M. & Plouffe L., Jr. (2004) Comparison of fracture, cardiovascular event, and breast cancer rates at 3 years in postmenopausal women with osteoporosis. J Am Geriatr Soc 52, 1543-8.
Singh A. R., Sivadas A., Sabharwal A., Vellarikal S. K., Jayarajan R., Verma A., Kapoor S., Joshi A., Scaria V. & Sivasubbu S. (2016) Chamber Specific Gene Expression Landscape of the Zebrafish Heart. PLoS One 11, e0147823.
Song C X, Szulwach K E, Fu Y et al. Selective chemical labeling reveals the genome-wide distribution of 5-hydroxymethylcytosine. Nat Biotechol 2011; 29:68-72
Stewart C. M., Kothari P. D., Mouliere F., Mair R., Somnay S., Benayed R., Zehir A., Weigelt B., Dawson S. J., Arcila M. E., Berger M. F. & Tsui D. W. (2018) The value of cell-free DNA for molecular pathology. J Pathol 244, 616-27.
Taglauer E. S., Wilkins-Haug L. & Bianchi D. W. (2014) Review: cell-free fetal DNA in the maternal circulation as an indication of placental health and disease. Placenta 35 Suppl, S64-8.
Tahiliani M, Koh K P, Shen Y et al. Conversion of 5-methylcytosine to 5-hydroxymethylcytosine in mammalian DNA by MLL partner TET1. Science 2009; 324:930-5
Takayanagi T., Forrester S. J., Kawai T., Obama T., Tsuji T., Elliott K. J., Nuti E., Rossello A., Kwok H. F., Scalia R., Rizzo V. & Eguchi S. (2016) Vascular ADAM17 as a Novel Therapeutic Target in Mediating Cardiovascular Hypertrophy and Perivascular Fibrosis Induced by Angiotensin II. Hypertension 68, 949-55.
Tan C H, Denny C H, Cheal N E et al. Alcohol use and binge drinking among women of childbearing age—United States, 2011-2013. MMWR Morb Mortal Wkly Rep 2015; 64:1042-6
Tjoa M. L., Cindrova-Davies T., Spasic-Boskovic O., Bianchi D. W. & Burton G. J. (2006)
Trophoblastic oxidative stress and the release of cell-free feto-placental DNA. Am J Pathol 169, 400-4.
Vallaster M., Vallaster C. D. & Wu S. M. (2012) Epigenetic mechanisms in cardiac development and disease. Acta Biochim Biophys Sin (Shanghai) 44, 92-102.
van Gelder M. M., Van Bennekom C. M., Louik C., Werler M. M., Roeleveld N. & Mitchell A. A. (2015) Maternal hypertensive disorders, antihypertensive medication use, and the risk of birth defects: a case-control study. BJOG 122, 1002-9.
von Oettingen J. E., Tan W. H. & Dauber A. (2014) Skeletal dysplasia, global developmental delay, and multiple congenital anomalies in a 5-year-old boy-report of the second family with B3GAT3 mutation and expansion of the phenotype. Am J Med Genet A 164A, 1580-6.
Wang X., Oka T., Chow F. L., Cooper S. B., Odenbach J., Lopaschuk G. D., Kassiri Z. & Fernandez-Patron C. (2009) Tumor necrosis factor-alpha-converting enzyme is a key regulator of agonist-induced cardiac hypertrophy and fibrosis. Hypertension 54, 575-82.
Wataganara T., Gratacos E., Jani J., Becker J., Lewi L., Sullivan L. M., Bianchi D. W. & Deprest J. A. (2005) Persistent elevation of cell-free fetal DNA levels in maternal plasma after selective laser coagulation of chorionic plate anastomoses in severe midgestational twin-twin transfusion syndrome. Am J Obstet Gynecol 192, 604-9.
Ye J., Llorian M., Cardona M., Rongvaux A., Moubarak R. S., Comella J. X., Bassel-Duby R., Flavell R. A., Olson E. N., Smith C. W. & Sanchis D. (2013) A pathway involving HDAC5, cFLIP and caspases regulates expression of the splicing regulator polypyrimidine tract binding protein in the heart. J Cell Sci 126, 1682-91.
Yeh W. C., Itie A., Elia A. J., Ng M., Shu H. B., Wakeham A., Mirtsos C., Suzuki N., Bonnard M., Goeddel D. V. & Mak T. W. (2000) Requirement for Casper (c-FLIP) in regulation of death receptor-induced apoptosis and embryonic development. Immunity 12, 633-42.
Zeisel S H, da Costa K A. Choline: an essential nutrient for public health. Nut Rev 2009; 67:615-23
Zheng D. Y., Zhao J., Yang J. M., Wang M. & Zhang X. T. (2016) Enhanced ADAM17 expression is associated with cardiac remodeling in rats with acute myocardial infarction. Life Sci 151, 61-9.
Zolotukhina T. V., Shilova N. V. & Voskoboeva E. Y. (2005) Analysis of cell-free fetal DNA in plasma and serum of pregnant women. J Histochem Cytochem 53, 297-9.

1. A method of predicting or diagnosing congenital heart defect (CHD) in a subject in need thereof, wherein the method comprises assaying a biological sample, obtained from the subject, comprising cell-free (cf) nucleic acids to determine frequency or percentage of cytosine methylation at one or more loci throughout genome; and comparing the cytosine methylation level of the sample to cytosine methylation of a control sample.

2. The method of claim 1, wherein the method further comprises using artificial intelligence (AI) techniques.

3. The method of claim 1, wherein the method further comprises using (AI) techniques comprising one or more of the following machine learning algorithms: Random Forest (RF), Support Vector Machine (SVM), Linear Discriminant Analysis (LDA), Prediction of Analysis for Microarrays (PAM), Generalized Linear Model (GLM), or deep learning (DL).

4. The method of claim 1, wherein the method further comprises calculating the subject's risk of developing CHD.

5. The method of claim 1, wherein the control sample is from one or more normal (healthy) patients or from one or more patients diagnosed with CHD.

6. The method of claim 1, wherein the biological sample comprises body fluid.

7. The method of claim 1, wherein the biological sample comprises blood, plasma, serum, urine, saliva, sputum, sweat, tears, genital secretion including cervical secretion, amniotic fluid, placental tissue, and umbilical cord blood obtained at birth.

8. The method of claim 1, wherein the cf nucleic acids comprise cell-free fetal (cfF) nucleic acids.

9. The method of claim 1, wherein the biological sample comprises cfF nucleic acids from first trimester, second trimester, or third trimester of pregnancy.

10. The method of claim 1, wherein the cf nucleic acids comprise DNA.

11. The method of claim 1, wherein the one or more loci comprise one or more loci from Tables 1-6.

12. The method of claim 1, wherein the one or more loci comprise at least two, at least three, at least four, at least five, at least six, at least seven, at least eight, at least nine, at least 10 loci from Tables 1-6.

13. The method of claim 1, wherein the one or more loci comprise an AUC (with 95% CI) of greater than 0.70, 0.75, 0.80, 0.85, 0.90, 0.91, 0.92, 0.93, 0.94, 0.95, 0.96, or 0.97.

14. The method of claim 1, wherein the assay is a bisulfite-based methylation assay or a whole-genome methylation assay.

15. The method of claim 1, wherein the one or more loci comprise cg06301252, cg02807450, or cg12900404.

16. The method of claim 1, wherein the one or more loci comprise cg04761177, cg21431091, cg01263077, cg09853933, cg27142059, cg16551159, cg14957943, cg06978680, or cg12592721.

17. The method of claim 1, wherein the method further comprises treating the CHD.

18. The method of claim 1, wherein the method further comprises treating the subject by administering medication and/or performing surgery on the subject.
